(19) United States
(12) Patent Application Publication
(10) Pub. No.: US 2018/0042234 A1
(43) Pub. Date: Feb. 15, 2018

(54) FUNGICIDAL PYRAZOLES

(71) Applicant: E I DU PONT DE NEMOURS AND COMPANY, Wilmington, DE (US)

(72) Inventors: Andrew Edmund Taggi, Newark, DE (US); Jeffrey Keith Long, Wilmington, DE (US)

(21) Appl. No.: 15/551,352

(22) PCT Filed: Mar. 16, 2016
(86) PCT No.: PCT/US16/22557

§ 371 Date: Aug. 16, 2017

Related U.S. Application Data
(60) Provisional application No. 62/135,288, filed on Mar. 19, 2015.

(51) Int. Cl. A01N 43/56; C07D 231/18; A01N 43/80; C07D 231/12; C07D 413/04; C07D 231/38; C07D 405/04

(52) U.S. Cl. CPC A01N 43/56; C07D 231/38; C07D 231/18; C07D 405/04; C07D 231/12; C07D 413/04; A01N 43/80

(57) ABSTRACT

Disclosed are compounds of Formula 1, including all geometric and stereoisomers, N-oxides, and salts thereof,

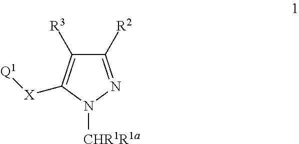

wherein
Q¹, X, R¹, R^{1a}, R² and R³ are as defined in the disclosure.

Also disclosed are compositions containing the compounds of Formula 1 and methods for controlling plant disease caused by a fungal pathogen comprising applying an effective amount of a compound or a composition of the invention.

### FIELD OF THE INVENTION

This invention relates to certain pyrazoles, their N-oxides, salts and compositions, and methods of their use as fungicides.

### BACKGROUND OF THE INVENTION

The control of plant diseases caused by fungal plant pathogens is extremely important in achieving high crop efficiency. Plant disease damage to ornamental, vegetable, field, cereal, and fruit crops can cause significant reduction in productivity and thereby result in increased costs to the consumer. Many products are commercially available for these purposes, but the need continues for new compounds which are more effective, less costly, less toxic, environmentally safer or have different sites of action.

PCT Patent Publications WO 2009/137538, WO 2009/137651, WO 2010/101973, WO 2012/023143, WO 2012/030922, WO 2012/031061, WO 2013/116251, WO 2013/126283 and WO 2013/192126 disclose pyrazole derivatives and their use as fungicides.

### SUMMARY OF THE INVENTION

This invention is directed to compounds of Formula 1 (including all stereoisomers), N-oxides, and salts thereof, agricultural compositions containing them and their use as fungicides:

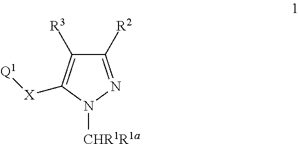

wherein
Q¹ is a phenyl ring or a naphthalenyl ring system, each ring or ring system optionally substituted with up to 5 substituents independently selected from R⁴; or a 5- to 6-membered fully unsaturated heterocyclic ring or an 8- to 10-membered heteroaromatic bicyclic ring system, each ring or ring system containing ring members selected from carbon atoms and 1 to 4 heteroatoms independently selected from up to 2 O, up to 2 S and up to 4 N atoms, wherein up to 3 carbon ring members are independently selected from C(═O) and C(═S), and the sulfur atom ring members are independently selected from S(═O)ᵤ(═NR¹¹)ᵥ, each ring or ring system optionally substituted with up to 5 substituents independently selected from R⁴ on carbon atom ring members and selected from cyano, C₁-C₄ alkyl, C₂-C₄ alkenyl, C₂-C₄ alkynyl, C₃-C₆ cycloalkyl, C₂-C₄ alkoxyalkyl, C₁-C₄ alkoxy, C₂-C₄ alkylcarbonyl, C₂-C₄ alkoxycarbonyl, C₂-C₄ alkylaminoalkyl and C₃-C₄ dialkylaminoalkyl on nitrogen atom ring members;
X is O, S(═O)ₘ, NR⁵ or CR^{6a}OR^{6b};
R¹ is H, cyano, halogen, C₁-C₃ alkyl, C₁-C₃ haloalkyl, C₂-C₃ alkenyl, C₂-C₃ alkynyl, cyclopropyl, C₂-C₃ alkoxyalkyl, C₁-C₃ alkoxy or C₁-C₃ haloalkoxy;
R^{1a} is H; or
R^{1a} and R¹ are taken together with the carbon atom to which they are attached to form a cyclopropyl ring optionally substituted with up to 2 substituents independently selected from halogen and methyl;
R² is H, cyano, halogen, C₁-C₃ alkyl, C₁-C₃ haloalkyl, C₂-C₃ alkenyl, C₂-C₃ haloalkenyl, C₂-C₃ alkynyl, C₂-C₃ cyanoalkyl, C₁-C₃ hydroxyalkyl, C₁-C₃ alkoxy or C₁-C₃ alkylthio; or cyclopropyl optionally substituted with up to 2 substituents independently selected from halogen and methyl;
R³ is C₁-C₈ alkyl, C₁-C₈ haloalkyl, C₂-C₈ alkenyl, C₂-C₈ haloalkenyl, C₂-C₈ alkynyl, C₂-C₈ haloalkynyl, C₂-C₈ cyanoalkyl, C₁-C₈ hydroxyalkyl, C₁-C₈ nitroalkyl, C₂-C₈ alkoxyalkyl, C₂-C₈ haloalkoxyalkyl, C₄-C₁₀ cycloalkoxyalkyl, C₃-C₈ alkoxyalkoxyalkyl, C₂-C₈ alkylthioalkyl, C₂-C₈ haloalkylthioalkyl, C₂-C₈ alkylsulfinylalkyl, C₂-C₈ haloalkylsulfinylalkyl, C₂-C₈ alkylsulfonylalkyl, C₂-C₈ haloalkylsulfonylalkyl, C₃-C₈ alkylcarbonyl, C₃-C₈ alkylcarbonylalkyl, C₃-C₈ haloalkylcarbonylalkyl, C₃-C₈ alkoxycarbonylalkyl, C₃-C₈ haloalkoxycarbonylalkyl, C₂-C₈ alkylaminoalkyl, C₂-C₈ haloalkylaminoalkyl, C₃-C₈ dialkylaminoalkyl, C₃-C₈ alkylaminocarbonylalkyl, C₄-C₁₀ dialkylaminocarbonylalkyl, C₄-C₁₀ cycloalkylaminoalkyl, —CR^{7a}═NOR^{7b} or —(CH₂)ₙW; or C₃-C₈ cycloalkyl, C₃-C₈ cycloalkenyl or C₄-C₁₀ cycloalkylalkyl, each optionally substituted with up to 3 substituents independently selected from R⁸;
W is a 3- to 7-membered saturated or partially unsaturated heterocyclic ring containing ring members selected from carbon atoms and 1 to 4 heteroatoms independently selected from up to 2 O, up to 2 S and up to 3 N atoms, wherein up to 3 carbon atom ring members are independently selected from C(═O) and C(═S), the ring optionally substituted with up to 3 substituents independently selected from R⁹ on carbon atom ring members and R¹⁰ on nitrogen atom ring members;
each R⁴ is independently amino, cyano, halogen, hydroxy, nitro, C₁-C₈ alkyl, C₁-C₈ haloalkyl, C₂-C₈ alkenyl, C₂-C₈ haloalkenyl, C₂-C₈ alkynyl, C₂-C₈ haloalkynyl, C₁-C₈ nitroalkyl, C₂-C₈ nitroalkenyl, C₃-C₈ cycloalkyl, C₃-C₈ halocycloalkyl, C₁-C₈ alkylthio, C₁-C₈ haloalkylthio, C₁-C₈ alkylsulfinyl, C₁-C₈ haloalkylsulfinyl, C₁-C₈ alkylsulfonyl, C₁-C₈ haloalkylsulfonyl, C₁-C₈ alkoxy, C₁-C₈ haloalkoxy, C₂-C₈ alkenyloxy, C₂-C₈ haloalkenyloxy, C₃-C₈ alkynyloxy, C₃-C₈ haloalkynyloxy, C₄-C₁₂ cycloalkylalkoxy, C₂-C₈ alkylcarbonyloxy, C₂-C₈ alkylaminoalkoxy, C₃-C₈ dialkylaminoalkoxy, C₂-C₈ alkylcarbonyl, C₁-C₈ alkylamino, C₂-C₈ dialkylamino, C₂-C₈ alkylcarbonylamino, —CH(═O), —NHCH(═O), —SF₅ or —SC≡N;
R⁵ is H, C₁-C₃ alkyl, C₂-C₆ cyanoalkyl or C₂-C₆ alkoxyalkyl;
R^{6a} is H or C₁-C₆ alkyl;
R^{6b} is H, —CH(═O), C₂-C₆ alkoxyalkyl, C₂-C₆ alkylcarbonyl or C₂-C₆ alkoxycarbonyl;
R^{7a} is H, C₁-C₃ alkyl or C₁-C₃ haloalkyl;
R^{7b} is H, C₁-C₃ alkyl, C₂-C₄ alkenyl, C₂-C₄ alkynyl, C₁-C₃ haloalkyl, C₂-C₄ haloalkenyl, C₃-C₄ cycloalkyl, C₄-C₈ cycloalkylalkyl or C₃-C₄ halocycloalkyl;
each R⁸ is independently cyano, halogen, hydroxy, C₁-C₃ alkyl, C₁-C₃ haloalkyl, C₃-C₆ cycloalkyl, C₁-C₃ alkoxy, C₁-C₃ haloalkoxy or C₂-C₄ alkoxyalkyl;
each R⁹ is independently cyano, halogen, C₁-C₃ alkyl, C₁-C₃ haloalkyl, C₁-C₃ alkoxy, C₁-C₃ haloalkoxy or C₂-C₄ alkoxyalkyl;
each R¹⁰ is independently cyano, C₁-C₃ alkyl or C₁-C₃ alkoxy;
each R¹¹ is independently H, cyano, C₁-C₃ alkyl or C₁-C₃ haloalkyl;
each u and v are independently 0, 1 or 2 in each instance of S(═O)ᵤ(═NR¹¹)ᵥ, provided that the sum of u and v is 0, 1 or 2;
m is 0, 1 or 2; and
n is 0 or 1.

More particularly, this invention pertains to a compound of Formula 1 (including all stereoisomers), an N-oxide or a salt thereof.

This invention also relates to a fungicidal composition comprising (a) a compound of the invention (i.e. in a fungicidally effective amount); and (b) at least one additional component selected from the group consisting of surfactants, solid diluents and liquid diluents.

This invention also relates to a fungicidal composition comprising (a) a compound of the invention; and (b) at least one other fungicide (e.g., at least one other fungicide having a different site of action).

This invention further relates to a method for controlling plant diseases caused by fungal plant pathogens comprising applying to the plant or portion thereof, or to the plant seed, a fungicidally effective amount of a compound of the invention (e.g., as a composition described herein).

This invention also relates to a composition comprising a compound of Formula 1, an N-oxide, or a salt thereof, and at least one invertebrate pest control compound or agent.

### DETAILS OF THE INVENTION

As used herein, the terms “comprises,” “comprising,” “includes,” “including,” “has,” “having,” “contains,” “containing,” “characterized by” or any other variation thereof, are intended to cover a non-exclusive inclusion, subject to any limitation explicitly indicated. For example, a composition, mixture, process, method, article, or apparatus that comprises a list of elements is not necessarily limited to only those elements but may include other elements not expressly listed or inherent to such composition, mixture, process, method, article, or apparatus.

The transitional phrase “consisting of” excludes any element, step, or ingredient not specified. If in the claim, such would close the claim to the inclusion of materials other than those recited except for impurities ordinarily associated therewith. When the phrase “consisting of” appears in a clause of the body of a claim, rather than immediately following the preamble, it limits only the element set forth in that clause; other elements are not excluded from the claim as a whole.

The transitional phrase “consisting essentially of” is used to define a composition, method or apparatus that includes materials, steps, features, components, or elements, in addition to those literally disclosed, provided that these additional materials, steps, features, components, or elements do not materially affect the basic and novel characteristic(s) of the claimed invention. The term “consisting essentially of” occupies a middle ground between “comprising” and “consisting of”.

Where applicants have defined an invention or a portion thereof with an open-ended term such as “comprising,” it should be readily understood that (unless otherwise stated) the description should be interpreted to also describe such an invention using the terms “consisting essentially of” or “consisting of”.

Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Also, the indefinite articles “a” and “an” preceding an element or component of the invention are intended to be nonrestrictive regarding the number of instances (i.e. occurrences) of the element or component. Therefore “a” or “an” should be read to include one or at least one, and the singular word form of the element or component also includes the plural unless the number is obviously meant to be singular.

As referred to in the present disclosure and claims, “plant” includes members of Kingdom Plantae, particularly seed plants (Spermatopsida), at all life stages, including young plants (e.g., germinating seeds developing into seedlings) and mature, reproductive stages (e.g., plants producing flowers and seeds). Portions of plants include geotropic members typically growing beneath the surface of the growing medium (e.g., soil), such as roots, tubers, bulbs and corms, and also members growing above the growing medium, such as foliage (including stems and leaves), flowers, fruits and seeds.

As referred to herein, the term “seedling”, used either alone or in a combination of words means a young plant developing from the embryo of a seed.

As referred to herein, the term “broadleaf” used either alone or in words such as “broadleaf crop” means dicot or dicotyledon, a term used to describe a group of angiosperms characterized by embryos having two cotyledons.

As referred to in this disclosure, the terms “fungal pathogen” and “fungal plant pathogen” include pathogens in the Ascomycota, Basidiomycota and Zygomycota phyla, and the fungal-like Oomycota class that are the causal agents of a broad spectrum of plant diseases of economic importance, affecting ornamental, turf, vegetable, field, cereal and fruit crops. In the context of this disclosure, “protecting a plant from disease” or “control of a plant disease” includes preventative action (interruption of the fungal cycle of infection, colonization, symptom development and spore production) and/or curative action (inhibition of colonization of plant host tissues).

As used herein, the term mode of action (MOA) is as define by the Fungicide Resistance Action Committee (FRAC), and is used to distinguish fungicides according to their biochemical mode of action in the biosynthetic pathways of plant pathogens. FRAC-defined modes of actions include (A) nucleic acid synthesis, (B) mitosis and cell division, (C) respiration, (D) amino acid and protein synthesis, (E) signal transduction, (F) lipid synthesis and membrane integrity, (G) sterol biosynthesis in membranes, (H) cell wall biosynthesis, (I) melanin synthesis in cell wall, (P) host plant defense induction, (U) unknown mode of action, (NC) not classified and (M) multi-site contact activity. Each MOA (i.e. letters A through M) contain one or more subgroups (e.g., A includes subgroups A1, A2, A3 and A4) based either on individual validated target sites of action, or in cases where the precise target site is unknown, based on cross resistance profiles within a group or in relation to other groups. Each of these subgroups (e.g., A1, A2, A3 and A4) is assigned a FRAC code (a number and/or letter). For example, the FRAC code for subgroup A1 is 4. Additional information on target sites and FRAC codes can be obtained from publicly available databases maintained, for example, by FRAC.

As used herein, the term “cross resistance” refers to the phenomenon that occurs when a pathogen develops resistance to one fungicide and simultaneously becomes resistant to one or more other fungicides. These other fungicides are typically, but not always, in the same chemical class or have the same target site of action, or can be detoxified by the same mechanism.

Generally when a molecular fragment (i.e. radical) is denoted by a series of atom symbols (e.g., C, H, N, O and S) the implicit point or points of attachment will be easily recognized by those skilled in the art. In some instances herein, particularly when alternative points of attachment are possible, the point or points of attachment may be explicitly indicated by a hyphen (“-”). For example, “—SCN” indicates that the point of attachment is the sulfur atom (i.e. thiocyanato, not isothiocyanato).

As used herein, the term “alkylating agent” refers to a chemical compound in which a carbon-containing radical is bound through a carbon atom to a leaving group such as halide or sulfonate, which is displaceable by bonding of a nucleophile to said carbon atom. Unless otherwise indicated, the term “alkylating” does not limit the carbon-containing radical to an alkyl group; the carbon-containing radicals can include the variety of carbon-bound radicals specified for example by R² and R³.

In the above recitations, the term “alkyl”, used either alone or in compound words such as “alkylthio” or “haloalkyl” includes straight-chain or branched alkyl such as methyl, ethyl, n-propyl, i-propyl, or the different butyl, pentyl or hexyl isomers. “Alkenyl” includes straight-chain or branched alkene such as ethenyl, 1-propenyl, 2-propenyl, and the different butenyl, pentenyl and hexenyl isomers. “Alkenyl” also includes polyenes such as 1,2-propadienyl and 2,4-hexadienyl. “Alkynyl” includes straight-chain or branched alkynyl such as ethynyl, 1-propynyl, 2-propynyl and the different butynyl, pentynyl and hexynyl isomers. “Alkynyl” also includes moieties comprised of multiple triple bonds such as 2,5-hexadiynyl.

“Alkylamino” includes an NH radical substituted with straight-chain or branched alkyl. Examples of “alkylamino” include CH₃CH₂NH, CH₃CH₂CH₂NH and (CH₃)₂CHNH. Examples of “dialkylamino” include (CH₃)₂N, (CH₃CH₂)₂N and CH₃CH₂(CH₃)N. “Alkylaminoalkyl” denotes alkylamino substitution on alkyl. Examples of “alkylaminoalkyl” include CH₃NHCH₂, CH₃NHCH₂CH₂ and CH₃CH₂NHCH₂. Examples of “dialkylaminoalkyl” include (CH₃)₂NCH₂, CH₃CH₂(CH₃)NCH₂ and (CH₃)₂NCH₂CH₂.

“Alkoxy” includes, for example, methoxy, ethoxy, n-propyloxy, i-propyloxy and the different butyl, pentyl and hexyloxy isomers. “Alkoxyalkyl” denotes alkoxy substitution on alkyl. Examples of “alkoxyalkyl” include CH₃OCH₂, CH₃OCH₂CH₂, CH₃CH₂OCH₂, CH₃CH₂CH₂CH₂OCH₂ and CH₃CH₂OCH₂CH₂. “Alkenyloxy” includes straight-chain or branched alkenyl attached to and linked through an oxygen atom. Examples of “alkenyloxy” include H₂C═CHCH₂O, (CH₃)₂C═CHCH₂O, CH₃CH═CHCH₂O, CH₃CH═C(CH₃)CH₂O and H₂C═CHCH₂CH₂O. “Alkynyloxy” includes straight-chain or branched alkynyl attached to and linked through an oxygen atom. Examples of “alkynyloxy” include HCCCH₂O, CH₃CCCH₂O and CH₃CCCH₂CH₂O. “Alkoxyalkoxyalkyl” denotes alkoxyalkoxy substitution on alkyl. Examples of “alkoxyalkoxyalkyl” include CH₃OCH₂OCH₂CH₃OCH₂OCH₂CH₂ and CH₃CH₂OCH₂OCH₂.

“Alkylthio” includes branched or straight-chain alkylthio moieties such as methylthio, ethylthio, and the different propyl, butyl, pentyl and hexylthio isomers. “Alkylsulfinyl” includes both enantiomers of an alkylsulfinyl group. Examples of “alkylsulfinyl” include CH₃S(═O), CH₃CH₂S(═O), CH₃CH₂CH₂S(═O) and (CH₃)₂CHS(═O). Examples of “alkyl sulfonyl” include CH₃S(═O)₂, CH₃CH₂S(═O)₂, CH₃CH₂CH₂S(═O)₂ and (CH₃)₂CHS(═O)₂. “Alkylthioalkyl” denotes alkylthio substitution on alkyl. Examples of “alkylthioalkyl” include CH₃SCH₂, CH₃SCH₂CH₂, CH₃CH₂SCH₂, CH₃CH₂CH₂CH₂S CH₂ and CH₃CH₂SCH₂CH₂; “alkylsulfinylalkyl” and “alkylsulfonylalkyl” include the corresponding sulfoxides and sulfones, respectively.

The term “cycloalkyl” denotes a saturated carbocyclic ring consisting of between 3 to 8 carbon atoms linked to one another by single bonds. Examples of “cycloalkyl” include cyclopropyl, cyclobutyl, cyclopentyl and cyclohexyl. The term “cycloalkylalkyl” denotes cycloalkyl substitution on an alkyl group. Examples of “cycloalkylalkyl” include cyclopropylmethyl, cyclopentylethyl, and other cycloalkyl moieties bonded to straight-chain or branched alkyl. “Cycloalkylalkoxy” denotes cycloalkyl substitution on an alkoxy group. Examples of “cycloalkylalkoxy” include cyclopropylmethoxy, cyclopentylethoxy, and other cycloalkyl moieties bonded to straight-chain or branched alkoxy. The term “cycloalkoxyalkyl” denotes cycloalkoxy substitution on an alkyl group. Examples of “cycloalkoxyalkyl” include cyclopropyloxymethyl, cyclopentyloxyethyl, and other cycloalkoxy moieties bonded to straight-chain or branched alkyl. The term “cycloalkylaminoalkyl” denotes cycloalkylamino substitution on an alkyl group. Examples of “cycloalkylaminoalkyl” include cyclopropylaminomethyl, cyclopentylaminoethyl, and other cycloalkylamino moieties bonded to straight-chain or branched alkyl. “Cycloalkenyl” includes groups such as cyclopentenyl and cyclohexenyl as well as groups with more than one double bond such as 1,3- or 1,4-cyclohexadienyl.

“Cyanoalkyl” denotes an alkyl group substituted with one cyano group. Examples of “cyanoalkyl” include NCCH₂, NCCH₂CH₂ and CH₃CH(CN)CH₂. “Hydroxyalkyl” denotes an alkyl group substituted with one hydroxy group. Examples of “hydroxyalkyl” include HOCH₂, HOCH₂CH₂ and CH₃CH₂(OH)CH. “Nitroalkyl” denotes an alkyl group substituted with one nitro group. Examples of “nitroalkyl” include NO₂CH₂ and NO₂CH₂CH₂.

“Alkylcarbonyl” denotes straight-chain or branched alkyl bonded to a C(═O) moiety. Examples of “alkylcarbonyl” include CH₃C(═O), CH₃CH₂CH₂C(═O) and (CH₃)₂CHC(═O). Examples of “alkoxycarbonyl” include CH₃OC(═O), CH₃CH₂OC(═O), CH₃CH₂CH₂OC(═O), (CH₃)₂CHOC(═O) and the different pentyl or hexyloxycarbonyl isomers. The term “alkylcarbonyloxy” denotes straight-chain or branched alkyl bonded to a C(═O)O moiety. Examples of “alkylcarbonyloxy” include CH₃CH₂C(═O)O and (CH₃)₂CHC(═O)O. The term “alkoxycarbonylalkyl” denotes alkoxycarbonyl substitution on alkyl. Examples of “alkoxycarbonylalkyl” include CH₃CH₂OC(═O)CH₂, (CH₃)₂CHCH₂OC(═O)CH₂ and CH₃OC(═O)CH₂CH₂. The term “alkylcarbonylamino” denotes alkyl bonded to a C(═O)NH moiety. Examples of “alkylcarbonylamino” include CH₃C(═O)NH and CH₃CH₂C(═O)NH.

The term “halogen”, either alone or in compound words such as “halomethyl” or “haloalkyl”, includes fluorine, chlorine, bromine or iodine. Further, when used in compound words such as “haloalkyl”, said alkyl may be partially or fully substituted with halogen atoms which may be the same or different. Examples of “haloalkyl” include F₃C, ClCH₂, CF₃CH₂ and CF₃CCl₂. The terms “haloalkenyl”, “haloalkoxy”, “haloalkylthio”, “haloalkylsulfinyl” “haloalkylsulfonyl”, “halocycloalkyl” and the like are defined analogously to the term “haloalkyl”. Examples of “haloalkenyl” include Cl₂C═CHCH₂ and CF₃CH═CH. Examples of “haloalkoxy” include CF₃O, CCl₃CH₂O, F₂CHCH₂CH₂O and CF₃CH₂O. Examples of “haloalkylthio” include CCl₃S, CF₃S, CCl₃CH₂S and ClCH₂CH₂CH₂S. Examples of “haloalkylsulfinyl” include CF₃S(═O), CCl₃S(═O), CF₃CH₂S(═O) and CF₃CF₂S(═O). Examples of “haloalkylsulfonyl” include CF₃S(═O)₂, CCl₃S(═O)₂, CF₃CH₂S(═O)₂ and CF₃CF₂S(═O)₂. Examples of “halocycloalkyl” include chlorocyclopropyl, fluorocyclobutyl and chlorocyclohexyl.

The total number of carbon atoms in a substituent group is indicated by the prefix “Cᵢ-Cⱼ” where i and j are numbers from 1 to 12. For example, C₁-C₃ alkylsulfonyl designates methylsulfonyl through propylsulfonyl; C₂ alkoxyalkyl designates CH₃OCH₂; C₃ alkoxyalkyl designates, for example, CH₃OCH₂CH₂ or CH₃CH₂OCH₂; and C₄ alkoxyalkyl designates the various isomers of an alkyl group substituted with an alkoxy group containing a total of four carbon atoms, examples including CH₃CH₂CH₂OCH₂ and CH₃CH₂OCH₂CH₂.

The term “unsubstituted” in connection with a group such as a ring means the group does not have any substituents other than its one or more attachments to the remainder of Formula 1. The term “optionally substituted” means that the number of substituents can be zero. Unless otherwise indicated, optionally substituted groups may be substituted with as many optional substituents as can be accommodated by replacing a hydrogen atom with a non-hydrogen substituent on any available carbon or nitrogen atom. Commonly, the number of optional substituents (when present) range from 1 to 3. As used herein, the term “optionally substituted” is used interchangeably with the phrase “substituted or unsubstituted” or with the term “(un)substituted.”

The number of optional substituents may be restricted by an expressed limitation. For example, the phrase “optionally substituted with up to 3 substituents independently selected from R⁴” means that 0, 1, 2 or 3 substituents can be present (if the number of potential connection points allows). Similarly, the phrase “optionally substituted with up to 5 substituents independently selected from R⁴” means that 0, 1, 2, 3, 4 or 5 substituents can be present if the number of available connection points allows.

Unless otherwise indicated, a “ring” or “ring system” as a component of Formula 1 is carbocyclic (e.g., phenyl or naphthalenyl) or heterocyclic (e.g., pyridinyl). The term “ring system” denotes two or more fused rings. The term “ring member” refers to an atom or other moiety (e.g., C(═O), C(═S), S(═O) or S(═O)₂) forming the backbone of a ring or ring system.

The term “aromatic” indicates that each of the ring atoms of a fully unsaturated ring are essentially in the same plane and have a p-orbital perpendicular to the ring plane, and that (4n+2) π electrons, where n is a positive integer, are associated with the ring to comply with Hückel's rule. The term “nonaromatic” includes rings that are fully saturated as well as partially or fully unsaturated, provided that none of the rings are aromatic.

The terms “carbocyclic ring” or “carbocycle” denote a ring wherein the atoms forming the ring backbone are selected only from carbon. When a fully unsaturated carbocyclic ring satisfies Hückel's rule, then said ring is also called an “aromatic carbocyclic ring”. The term “saturated carbocyclic ring” refers to a ring having a backbone consisting of carbon atoms linked to one another by single bonds; unless otherwise specified, the remaining carbon valences are occupied by hydrogen atoms.

The terms “heterocyclic ring”, “heterocycle” or “heteroaromatic ring system” denote a ring or ring system in which at least one atom forming the ring backbone is not carbon (e.g., N, O or S). Typically a heterocyclic ring contains no more than 3 N atoms, no more than 2 O atoms and no more than 2 S atoms. Unless otherwise indicated, a heterocyclic ring can be a saturated, partially unsaturated or fully unsaturated ring. When a fully unsaturated heterocyclic ring satisfies Hückel's rule, then said ring is also called a “heteroaromatic ring” or “aromatic heterocyclic ring”. Unless otherwise indicated, heterocyclic rings can be attached through any available carbon or nitrogen by replacement of a hydrogen on said carbon or nitrogen.

In the context of the present invention when an instance of Q¹ comprises a phenyl or 6-membered heterocyclic ring (e.g., pyridinyl), the ortho, meta and para positions of each ring are relative to the connection of the ring to the remainder of Formula 1.

Compounds of this invention can exist as one or more stereoisomers. Stereoisomers are isomers of identical constitution but differing in the arrangement of their atoms in space and include enantiomers, diastereomers, cis- and trans-isomers (also known as geometric isomers) and atropisomers. Atropisomers result from restricted rotation about single bonds where the rotational barrier is high enough to permit isolation of the isomeric species. One skilled in the art will appreciate that one stereoisomer may be more active and/or may exhibit beneficial effects when enriched relative to the other stereoisomer(s) or when separated from the other stereoisomer(s). Additionally, the skilled artisan knows how to separate, enrich, and/or to selectively prepare said stereoisomers. For a comprehensive discussion of all aspects of stereoisomerism, see Ernest L. Eliel and Samuel H. Wilen, Stereochemistry of Organic Compounds, John Wiley & Sons, 1994.

Compounds of this invention can exist as one or more conformational isomers due to restricted rotation about the amide bond (e.g., C(═O)—N) in Formula 1. This invention comprises mixtures of conformational isomers. In addition, this invention includes compounds that are enriched in one conformer relative to others.

This invention comprises all stereoisomers, conformational isomers and mixtures thereof in all proportions as well as isotopic forms such as deuterated compounds.

One skilled in the art will appreciate that not all nitrogen containing heterocycles can form N-oxides since the nitrogen requires an available lone pair for oxidation to the oxide; one skilled in the art will recognize those nitrogen-containing heterocycles which can form N-oxides. One skilled in the art will also recognize that tertiary amines can form N-oxides. Synthetic methods for the preparation of N-oxides of heterocycles and tertiary amines are very well known by one skilled in the art including the oxidation of heterocycles and tertiary amines with peroxy acids such as peracetic and m-chloroperbenzoic acid (MCPBA), hydrogen peroxide, alkyl hydroperoxides such as t-butyl hydroperoxide, sodium perborate, and dioxiranes such as dimethyldioxirane. These methods for the preparation of N-oxides have been extensively described and reviewed in the literature, see for example: T. L. Gilchrist in Comprehensive Organic Synthesis, vol. 7, pp 748-750, S. V. Ley, Ed., Pergamon Press; M. Tisler and B. Stanovnik in Comprehensive Heterocyclic Chemistry, vol. 3, pp 18-20, A. J. Boulton and A. McKillop, Eds., Pergamon Press; M. R. Grimmett and B. R. T. Keene in Advances in Heterocyclic Chemistry, vol. 43, pp 149-161, A. R. Katritzky, Ed., Academic Press; M. Tisler and B. Stanovnik in Advances in Heterocyclic Chemistry, vol. 9, pp 285-291, A. R. Katritzky and A. J. Boulton, Eds., Academic Press; and G. W. H. Cheeseman and E. S. G. Werstiuk in Advances in Heterocyclic Chemistry, vol. 22, pp 390-392, A. R. Katritzky and A. J. Boulton, Eds., Academic Press.

One skilled in the art recognizes that because in the environment and under physiological conditions salts of chemical compounds are in equilibrium with their corresponding nonsalt forms, salts share the biological utility of the nonsalt forms. Thus a wide variety of salts of the compounds of Formula 1 are useful for control of plant diseases caused by fungal plant pathogens (i.e. are agriculturally suitable). The salts of the compounds of Formula 1 include acid-addition salts with inorganic or organic acids such as hydrobromic, hydrochloric, nitric, phosphoric, sulfuric, acetic, butyric, fumaric, lactic, maleic, malonic, oxalic, propionic, salicylic, tartaric, 4-toluenesulfonic or valeric acids. When a compound of Formula 1 contains an acidic moiety such as a carboxylic acid, salts also include those formed with organic or inorganic bases such as pyridine, triethylamine or ammonia, or amides, hydrides, hydroxides or carbonates of sodium, potassium, lithium, calcium, magnesium or barium. Accordingly, the present invention comprises compounds selected from Formula 1, N-oxides and agriculturally suitable salts thereof.

Compounds selected from Formula 1, stereoisomers, tautomers, N-oxides, and salts thereof, typically exist in more than one form, and Formula 1 thus includes all crystalline and non-crystalline forms of the compounds that Formula 1 represents. Non-crystalline forms include embodiments which are solids such as waxes and gums as well as embodiments which are liquids such as solutions and melts. Crystalline forms include embodiments which represent essentially a single crystal type and embodiments which represent a mixture of polymorphs (i.e. different crystalline types). The term “polymorph” refers to a particular crystalline form of a chemical compound that can crystallize in different crystalline forms, these forms having different arrangements and/or conformations of the molecules in the crystal lattice. Although polymorphs can have the same chemical composition, they can also differ in composition due to the presence or absence of co-crystallized water or other molecules, which can be weakly or strongly bound in the lattice. Polymorphs can differ in such chemical, physical and biological properties as crystal shape, density, hardness, color, chemical stability, melting point, hygroscopicity, suspensibility, dissolution rate and biological availability. One skilled in the art will appreciate that a polymorph of a compound represented by Formula 1 can exhibit beneficial effects (e.g., suitability for preparation of useful formulations, improved biological performance) relative to another polymorph or a mixture of polymorphs of the same compound represented by Formula 1. Preparation and isolation of a particular polymorph of a compound represented by Formula 1 can be achieved by methods known to those skilled in the art including, for example, crystallization using selected solvents and temperatures. For a comprehensive discussion of polymorphism see R. Hilfiker, Ed., Polymorphism in the Pharmaceutical Industry, Wiley-VCH, Weinheim, 2006.

Embodiments of the present invention as described in the Summary of the Invention include those described below. In the following Embodiments, Formula 1 includes stereoisomers, N-oxides and salts thereof, and reference to “a compound of Formula 1” includes the definitions of substituents specified in the Summary of the Invention unless further defined in the Embodiments.

### Embodiment 1

A compound of Formula 1 wherein Q¹ is a phenyl ring substituted with 1 to 3 substituents independently selected from R⁴; or a pyridinyl, pyrimidinyl, pyrazinyl or pyridazinyl ring, each ring optionally substituted with up to 3 substituents independently selected from R⁴.

### Embodiment 2

A compound of Embodiment 1 wherein Q¹ is a phenyl ring substituted with 1 to 3 substituents independently selected from R⁴; or a pyridinyl ring optionally substituted with up to 3 substituents independently selected from R⁴.

### Embodiment 3

A compound of Embodiment 2 wherein Q¹ is a phenyl ring substituted with 1 to 3 substituents independently selected from R⁴.

### Embodiment 4

A compound of Embodiment 3 wherein Q¹ is a phenyl ring substituted with 2 to 3 substituents independently selected from R⁴.

### Embodiment 5

A compound of Embodiment 4 wherein Q¹ is a phenyl ring substituted with 3 substituents independently selected from R⁴.

### Embodiment 6

A compound of Embodiment 4 wherein Q¹ is a phenyl ring substituted with 2 substituents independently selected from R⁴.

### Embodiment 7

A compound of Formula 1 or any one of Embodiments 1 through 6 wherein Q¹ is a phenyl ring substituted with at least one R⁴ substituent attached at an ortho position (relative to the connection of the Q¹ ring to the remainder of Formula 1).

### Embodiment 8

A compound of Formula 1 or any one of Embodiments 1 through 7 wherein Q¹ is a phenyl ring substituted with at least one R⁴ substituent attached at the para position (relative to the connection of the Q¹ ring to the remainder of Formula 1).

### Embodiment 9

A compound of Formula 1 or any one of Embodiments 1 through 8 wherein Q¹ is a phenyl ring substituted at the 2-, 4- and 6-positions with substituents independently selected from R⁴; or a phenyl ring substituted at the 2- and 4-positions with substituents independently selected from R⁴; or a phenyl ring substituted at the 2- and 6-positions with substituents independently selected from R⁴.

### Embodiment 10

A compound of Formula 1 or any one of Embodiments 1 through 9 wherein X is O, NR⁵ or CR^{6a}OR^{6b}.

### Embodiment 11

A compound of Embodiment 10 wherein X is O, NH or CHOH.

### Embodiment 12

A compound of Embodiment 11 wherein X is O or CHOH.

### Embodiment 13

A compound of Embodiment 11 wherein X is NH or CHOH.

### Embodiment 14

A compound of Embodiment 11 wherein X is O or NH.

### Embodiment 14a

A compound of Embodiment 11 wherein X is NH.

### Embodiment 15

A compound of Formula 1 or any one of Embodiments 1 through 14a wherein when R¹ is taken alone (i.e. not taken together with R^{1a}), then R¹ is H, C₁-C₃ alkyl, C₁-C₃ haloalkyl, cyclopropyl, C₁-C₃ alkoxy or C₁-C₃ haloalkoxy.

### Embodiment 16

A compound of Embodiment 15 wherein R¹ is H, C₁-C₃ alkyl, C₁-C₃ haloalkyl or C₁-C₃ alkoxy.

### Embodiment 17

A compound of Embodiment 16 wherein R¹ is H or C₁-C₃ alkyl.

### Embodiment 18

A compound of Embodiment 17 wherein R¹ is H or methyl.

### Embodiment 19

A compound of Embodiment 18 wherein R¹ is H.

### Embodiment 20

A compound of Formula 1 or any one of Embodiments 1 through 19 wherein R¹ is taken alone.

### Embodiment 21

A compound of Formula 1 or any one of Embodiments 1 through 20 wherein R^{1a} is H.

### Embodiment 22

A compound of Formula 1 or any one of Embodiments 1 through 21 wherein when R^{1a} and R¹ are taken together with the carbon atom to which they are attached to form a ring, then said ring is cyclopropyl (i.e. unsubstituted).

### Embodiment 23

A compound of Formula 1 or any one of Embodiments 1 through 22 wherein R² is cyano, halogen, C₁-C₂ alkyl, halomethyl, cyanomethyl, hydroxymethyl, methoxy or methylthio; or cyclopropyl optionally substituted with up to 2 substituents independently selected from halogen and methyl.

### Embodiment 24

A compound of Embodiment 23 wherein R² is Br, Cl, I or C₁-C₂ alkyl.

### Embodiment 25

A compound of Embodiment 24 wherein R² is Br, Cl or methyl.

### Embodiment 26

A compound of Embodiment 25 wherein R² is methyl.

### Embodiment 27

A compound of Formula 1 or any one of Embodiments 1 through 26 wherein R³ is C₁-C₆ alkyl, C₁-C₆ haloalkyl, C₂-C₆ alkenyl, C₂-C₆ haloalkenyl, C₂-C₆ alkynyl, C₂-C₆ cyanoalkyl, C₂-C₆ alkoxyalkyl, C₂-C₆ haloalkoxyalkyl, C₄-C₁₀ cycloalkoxyalkyl, C₃-C₆ alkoxyalkoxyalkyl, C₂-C₆ alkylthioalkyl, C₂-C₆ alkylsulfinylalkyl, C₂-C₆ haloalkylsulfinylalkyl, C₂-C₆ alkylsulfonylalkyl, C₂-C₆ haloalkylsulfonylalkyl, C₃-C₆ alkylcarbonylalkyl, C₃-C₆ haloalkylcarbonylalkyl, C₃-C₆ alkoxycarbonylalkyl, C₂-C₆ alkylaminoalkyl, C₃-C₆ dialkylaminoalkyl, C₃-C₆ alkylaminocarbonylalkyl or —(CH₂)ₙW; or C₃-C₆ cycloalkyl, C₃-C₆ cycloalkenyl or C₄-C₇ cycloalkylalkyl, each optionally substituted with up to 3 substituents independently selected from R⁸.

### Embodiment 28

A compound of Embodiment 27 wherein R³ is C₁-C₆ alkyl, C₁-C₆ haloalkyl, C₂-C₆ alkenyl, C₂-C₆ haloalkenyl, C₂-C₆ alkoxyalkyl, C₃-C₆ alkoxyalkoxyalkyl, C₂-C₆ alkylthioalkyl, C₂-C₆ alkylsulfinylalkyl, C₂-C₆ haloalkylsulfinylalkyl, C₂-C₆ alkylsulfonylalkyl, C₂-C₆ haloalkylsulfonylalkyl, C₃-C₆ alkylcarbonylalkyl, C₃-C₆ haloalkylcarbonylalkyl, C₃-C₆ alkoxycarbonylalkyl or —(CH₂)ₙW; or C₃-C₆ cycloalkyl, C₃-C₆ cycloalkenyl or C₄-C₇ cycloalkylalkyl, each optionally substituted with up to 2 substituents independently selected from R⁸.

### Embodiment 29

A compound of Embodiment 28 wherein R³ is C₁-C₆ alkyl, C₁-C₆ haloalkyl, C₂-C₆ alkenyl, C₂-C₆ alkoxyalkyl or —(CH₂)ₙW; or C₃-C₆ cycloalkyl, C₃-C₆ cycloalkenyl or C₄-C₇ cycloalkylalkyl, each optionally substituted with up to 1 substituent selected from R⁸.

### Embodiment 30

A compound of Embodiment 29 wherein R³ is C₁-C₆ alkyl, C₁-C₆ haloalkyl, C₂-C₆ alkenyl or —(CH₂)ₙW; or C₃-C₆ cycloalkyl, C₃-C₆ cycloalkenyl or C₄-C₇ cycloalkylalkyl, each optionally substituted with up to 1 substituent selected from R⁸.

### Embodiment 31

A compound of Embodiment 30 wherein R³ is C₁-C₆ alkyl, C₁-C₆ haloalkyl, C₂-C₆ alkenyl; or C₃-C₆ cycloalkyl, C₃-C₆ cycloalkenyl or C₄-C₇ cycloalkylalkyl, each optionally substituted with up to 1 substituent selected from R⁸.

### Embodiment 32

A compound of Embodiment 31 wherein R³ is C₁-C₆ alkyl, C₁-C₆ haloalkyl, C₂-C₆ alkenyl, C₃-C₆ cycloalkyl, C₃-C₆ cycloalkenyl or C₄-C₇ cycloalkylalkyl.

### Embodiment 33

A compound of Formula 1 or any one of Embodiments 1 through 30 wherein W is a 5- to 6-membered saturated or partially unsaturated heterocyclic ring containing ring members selected from carbon atoms and 1 to 3 heteroatoms independently selected from up to 2 O, up to 2 S and up to 3 N atoms, wherein up to 2 carbon atom ring members are selected from C(═O), the ring optionally substituted with up to 3 substituents independently selected from R⁹ on carbon atom ring members and R¹⁰ on nitrogen atom ring members.

### Embodiment 34

A compound of Embodiment 33 wherein W is a 5- to 6-membered saturated or partially unsaturated heterocyclic ring containing ring members selected from carbon atoms and 1 to 3 heteroatoms independently selected from up to 2 O, up to 2 S and up to 3 N atoms, the ring optionally substituted with up to 2 substituents independently selected from R⁹ on carbon atom ring members and R¹⁰ on nitrogen atom ring members.

### Embodiment 35

A compound of Embodiment 34 wherein W is a 5- to 6-membered saturated or partially unsaturated heterocyclic ring containing ring members selected from carbon atoms and 1 to 2 heteroatoms independently selected from up to 2 O, up to 2 S and up to 2 N atoms, the ring optionally substituted with up to 2 substituents independently selected from R⁹ on carbon atom ring members and R¹⁰ on nitrogen atom ring members.

### Embodiment 36

A compound of Formula 1 or any one of Embodiments 1 through 35 wherein W is tetrahydrofuranyl, tetrahydrothiophenyl, pyrrolidinyl, 1,3-dioxolanyl, 1,3-oxathiolanyl, 1,3-dithiolanyl, tetrahydro-2H-thiopyranyl, tetrahydropyranyl, tetrahydrothiopyranyl, piperidinyl, 1,3-dioxanyl, 1,3-oxathianyl or 1,3-dithianyl, each optionally substituted with up to 2 substituents independently selected from R⁹ on carbon atom ring members and R¹⁰ on nitrogen atom ring members.

### Embodiment 37

A compound of Formula 1 or any one of Embodiments 1 through 36 wherein each R⁴ is independently cyano, halogen, methyl, halomethyl, cyclopropyl, methylthio, C₁-C₄ alkoxy, C₁-C₄ haloalkoxy, C₂-C₆ alkenyloxy, C₂-C₆ haloalkenyloxy, C₂-C₆ alkynyloxy, C₃-C₆ haloalkynyloxy, C₄-C₆ cycloalkylalkoxy or C₂-C₆ alkylcarbonyloxy.

### Embodiment 38

A compound of Embodiment 37 wherein each R⁴ is independently cyano, halogen, methyl, C₁-C₄ alkoxy, C₁-C₄ haloalkoxy, C₂-C₆ alkenyloxy, C₂-C₆ haloalkenyloxy, C₂-C₆ alkynyloxy, C₃-C₆ haloalkynyloxy or C₄-C₆ cycloalkylalkoxy.

### Embodiment 39

A compound of Embodiment 38 wherein each R⁴ is independently cyano, halogen, methyl, C₁-C₄ alkoxy, C₂-C₆ alkynyloxy or C₄-C₆ cycloalkylalkoxy.

### Embodiment 40

A compound of Embodiment 39 wherein each R⁴ is independently cyano, halogen, methyl, methoxy or C₂-C₄ alkynyloxy.

### Embodiment 41

A compound of Embodiment 40 wherein each R⁴ is independently halogen.

### Embodiment 42

A compound of Embodiment 41 wherein each R⁴ is independently Cl, F or Br.

### Embodiment 43

A compound of Embodiment 42 wherein each R⁴ is independently Cl or F.

### Embodiment 44

A compound of Formula 1 or any one of Embodiments 1 through 43 wherein R⁵ is H, methyl, cyanomethyl or C₂-C₃ alkoxyalkyl.

### Embodiment 45

A compound of Embodiment 44 wherein R⁵ is H.

### Embodiment 46

A compound of Formula 1 or any one of Embodiments 1 through 45 wherein R^{6a} is H or methyl.

### Embodiment 47

A compound of Embodiment 46 wherein R^{6a} is H.

### Embodiment 48

A compound of Formula 1 or any one of Embodiments 1 through 47 wherein R^{6b} is H, —CH(═O), C₂-C₃ alkoxyalkyl, C₂-C₄ alkylcarbonyl or C₂-C₄ alkoxycarbonyl.

### Embodiment 49

A compound of Embodiment 48 wherein R^{6b} is H, —CH(═O), methylcarbonyl or methoxycarbonyl.

### Embodiment 50

A compound of Embodiment 49 wherein R^{6b} is H.

### Embodiment 51

A compound of Formula 1 or any one of Embodiments 1 through 50 wherein R^{7a} is H or methyl.

### Embodiment 52

A compound of Formula 1 or any one of Embodiments 1 through 51 wherein R^{7b} is H or methyl.

### Embodiment 53

A compound of Formula 1 or any one of Embodiments 1 through 52 wherein each R⁸ is independently halogen, methyl, halomethyl, cyclopropyl, methoxy or C₂-C₄ alkoxyalkyl.

### Embodiment 54

A compound of Embodiment 53 wherein each R⁸ is independently halogen, methyl, halomethyl or methoxy.

### Embodiment 55

A compound of Embodiment 54 wherein each R⁸ is independently halogen, methyl, CF₃ or methoxy.

### Embodiment 56

A compound of Formula 1 or any one of Embodiments 1 through 55 wherein each R⁹ is independently halogen, methyl, halomethyl, methoxy or C₂-C₄ alkoxyalkyl.

### Embodiment 57

A compound of Embodiment 56 wherein each R⁹ is independently halogen, methyl, CF₃ or methoxy.

### Embodiment 58

A compound of Embodiment 57 wherein each R⁹ is independently methyl or methoxy.

### Embodiment 59

A compound of Formula 1 or any one of Embodiments 1 through 58 wherein each R¹⁰ is methyl.

### Embodiment 60

A compound of Formula 1 or any one of Embodiments 1 through 59 wherein m is 0.

### Embodiment 61

A compound of Formula 1 or any one of Embodiments 1 through 60 wherein each n is 1.

### Embodiment 62

A compound of Formula 1 or any one of Embodiments 1 through 60 wherein each n is 0.

Embodiments of this invention, including Embodiments 1-62 above as well as any other embodiments described herein, can be combined in any manner, and the descriptions of variables in the embodiments pertain not only to the compounds of Formula 1 but also to the starting compounds and intermediate compounds useful for preparing the compounds of Formula 1 unless further defined in the Embodiments. In addition, embodiments of this invention, including Embodiments 1-62 above as well as any other embodiments described herein, and any combination thereof, pertain to the compositions and methods of the present invention. Combinations of Embodiments 1-62 are illustrated by:

### Embodiment A

A compound of Formula 1 wherein
Q¹ is a phenyl ring substituted with 1 to 3 substituents independently selected from R⁴; or a pyridinyl, pyrimidinyl, pyrazinyl or pyridazinyl ring, each ring optionally substituted with up to 3 substituents independently selected from R⁴;
X is O, NR⁵ or CR^{6a}OR^{6b};
R¹ is H or C₁-C₃ alkyl;
R^{1a} is H;
R² is Br, Cl or methyl;
R³ is C₁-C₆ alkyl, C₁-C₆ haloalkyl, C₂-C₆ alkenyl, C₂-C₆ haloalkenyl, C₂-C₆ alkynyl, C₂-C₆ cyanoalkyl, C₂-C₆ alkoxyalkyl, C₂-C₆ haloalkoxyalkyl, C₄-C₁₀ cycloalkoxyalkyl, C₃-C₆ alkoxyalkoxyalkyl, C₂-C₆ alkylthioalkyl, C₂-C₆ alkylsulfinylalkyl, C₂-C₆ haloalkylsulfinylalkyl, C₂-C₆ alkylsulfonylalkyl, C₂-C₆ haloalkylsulfonylalkyl, C₃-C₆ alkylcarbonylalkyl, C₃-C₆ haloalkylcarbonylalkyl, C₃-C₆ alkoxycarbonylalkyl, C₂-C₆ alkylaminoalkyl, C₃-C₆ dialkylaminoalkyl, C₃-C₆ alkylaminocarbonylalkyl or —(CH₂)ₙW; or C₃-C₆ cycloalkyl, C₃-C₆ cycloalkenyl or C₄-C₇ cycloalkylalkyl, each optionally substituted with up to 3 substituents independently selected from R⁸;
W is a 5- to 6-membered saturated or partially unsaturated heterocyclic ring containing ring members selected from carbon atoms and 1 to 3 heteroatoms independently selected from up to 2 O, up to 2 S and up to 3 N atoms, wherein up to 2 carbon atom ring members are selected from C(═O), the ring optionally substituted with up to 3 substituents independently selected from R⁹ on carbon atom ring members and R¹⁰ on nitrogen atom ring members;
each R⁴ is independently halogen;
R⁵ is H, methyl, cyanomethyl or C₂-C₃ alkoxyalkyl;
R^{6a} is H or methyl;
R^{6b} is H;
each R⁸ is independently halogen, methyl, halomethyl, cyclopropyl, methoxy or C₂-C₄ alkoxyalkyl;
each R⁹ is independently halogen, methyl, halomethyl, methoxy or C₂-C₄ alkoxyalkyl; and
each R¹⁰ is methyl.

### Embodiment B

A compound of Embodiment A wherein
Q¹ is a phenyl ring substituted with 1 to 3 substituents independently selected from R⁴;
X is O, NH or CHOH;
R¹ is H;
R² is methyl;
R³ is C₁-C₆ alkyl, C₁-C₆ haloalkyl, C₂-C₆ alkenyl, C₂-C₆ haloalkenyl, C₂-C₆ alkoxyalkyl, C₃-C₆ alkoxyalkoxyalkyl, C₂-C₆ alkylthioalkyl, C₂-C₆ alkylsulfinylalkyl, C₂-C₆ haloalkylsulfinylalkyl, C₂-C₆ alkylsulfonylalkyl, C₂-C₆ haloalkylsulfonylalkyl, C₃-C₆ alkylcarbonylalkyl, C₃-C₆ haloalkylcarbonylalkyl, C₃-C₆ alkoxycarbonylalkyl or —(CH₂)ₙW; or C₃-C₆ cycloalkyl, C₃-C₆ cycloalkenyl or C₄-C₇ cycloalkylalkyl, each optionally substituted with up to 2 substituents independently selected from R⁸;
W is a 5- to 6-membered saturated or partially unsaturated heterocyclic ring containing ring members selected from carbon atoms and 1 to 3 heteroatoms independently selected from up to 2 O, up to 2 S and up to 3 N atoms, the ring optionally substituted with up to 2 substituents independently selected from R⁹ on carbon atom ring members and R¹⁰ on nitrogen atom ring members;
each R⁴ is independently Cl, F or Br;
each R⁸ is independently halogen, methyl, halomethyl or methoxy; and
each R⁹ is independently halogen, methyl, CF₃ or methoxy.

### Embodiment C

A compound of Embodiment B wherein
Q¹ is a phenyl ring substituted at the 2-, 4- and 6-positions with substituents independently selected from R⁴; or a phenyl ring substituted at the 2- and 4-positions with substituents independently selected from R⁴; or a phenyl ring substituted at the 2- and 6-positions with substituents independently selected from R⁴;
R³ is C₁-C₆ alkyl, C₁-C₆ haloalkyl, C₂-C₆ alkenyl, C₂-C₆ alkoxyalkyl or —(CH₂)ₙW; or C₃-C₆ cycloalkyl, C₃-C₆ cycloalkenyl or C₄-C₇ cycloalkylalkyl, each optionally substituted with up to 1 substituent selected from R⁸; and
W is a 5- to 6-membered saturated or partially unsaturated heterocyclic ring containing ring members selected from carbon atoms and 1 to 2 heteroatoms independently selected from up to 2 O, up to 2 S and up to 2 N atoms, the ring optionally substituted with up to 2 substituents independently selected from R⁹ on carbon atom ring members and R¹⁰ on nitrogen atom ring members.

### Embodiment D

A compound of Formula 1 wherein
Q¹ is a phenyl ring substituted at the 2-, 4- and 6-positions with substituents independently selected from R⁴; or a phenyl ring substituted at the 2- and 4-positions with substituents independently selected from R⁴; or a phenyl ring substituted at the 2- and 6-positions with substituents independently selected from R⁴;
X is O, NH or CHOH;
R¹ is H;
R^{1a} is H;
R² is Br, Cl, I or C₁-C₂ alkyl;
R³ is C₁-C₆ alkyl, C₁-C₆ haloalkyl, C₂-C₆ alkenyl; or C₃-C₆ cycloalkyl, C₃-C₆ cycloalkenyl or C₄-C₇ cycloalkylalkyl, each optionally substituted with up to 1 substituent selected from R⁸;
each R⁴ is independently cyano, halogen, methyl, methoxy or C₂-C₄ alkynyloxy; and
each R⁸ is halogen, methyl, halomethyl or methoxy.

### Embodiment E

A compound of Embodiment D wherein
X is NH or CHOH;
R² is methyl;
R³ is C₁-C₆ alkyl, C₁-C₆ haloalkyl, C₂-C₆ alkenyl, C₃-C₆ cycloalkyl, C₃-C₆ cycloalkenyl or C₄-C₇ cycloalkylalkyl; and
each R⁴ is independently Cl, F or Br.

Specific embodiments include compounds of Formula 1 selected from the group consisting of:
α-(2-chloro-4-fluorophenyl)-4-cyclohexyl-1,3-dimethyl-1H-pyrazole-5-methanol (Compound 11);
4-cyclohexyl-α-(2,4-difluorophenyl)-1,3-dimethyl-1H-pyrazole-5-methanol (Compound 12);
N-(4-chloro-2,6-difluorophenyl)-1,3-dimethyl-4-(1-methyl-1-propen-1-yl)-1H-pyrazol-5-amine (Compound 24);
N-(4-chloro-2,6-difluorophenyl)-4-cyclohexyl-1,3-dimethyl-1H-pyrazol-5-amine (Compound 46); and
N-(4-chloro-2,6-difluorophenyl)-4-cyclopenyl-1,3-dimethyl-1H-pyrazol-5-amine (Compound 52).

This invention provides a fungicidal composition comprising a compound of Formula 1 (including all stereoisomers, N-oxides, and salts thereof), and at least one other fungicide. Of note as embodiments of such compositions are compositions comprising a compound corresponding to any of the compound embodiments described above.

This invention provides a fungicidal composition comprising a compound of Formula 1 (including all stereoisomers, N-oxides, and salts thereof) (i.e. in a fungicidally effective amount), and at least one additional component selected from the group consisting of surfactants, solid diluents and liquid diluents. Of note as embodiments of such compositions are compositions comprising a compound corresponding to any of the compound embodiments described above.

This invention provides a method for controlling plant diseases caused by fungal plant pathogens comprising applying to the plant or portion thereof, or to the plant seed, a fungicidally effective amount of a compound of Formula 1 (including all stereoisomers, N-oxides, and salts thereof). Of note as embodiments of such methods are methods comprising applying a fungicidally effective amount of a compound corresponding to any of the compound embodiments describe above. Of particular note are embodiments where the compounds are applied as compositions of this invention.

One or more of the following methods and variations as described in Schemes 1-21 can be used to prepare the compounds of Formula 1. The definitions of Q¹, X, R¹, R^{1a}, R², R³, R⁵, m and R^{6a} in the compounds of Formulae 1-21 below are as defined above in the Summary of the Invention unless otherwise noted. Formulae 1a, 1b, 1c, 1d, 1e, 1f and 1g are various subsets of Formula 1. Substituents for each subset formula are as defined for its parent formula unless otherwise noted

As shown in Scheme 1, compounds of Formula 1a (i.e. Formula 1 wherein X is CR^{6a}OR^{6b} and R^{6b} is H) can be prepared by contacting compounds of Formula 2 (e.g., aldehydes for R^{6a} being H or ketones for R^{6a} being alkyl) with organometallic reagents of formula Q¹-M¹ wherein M¹ is MgX¹, Li or ZnX¹ and X¹ is Br, Cl or I. The reaction typically is carried out in a suitable solvent such as tetrahydrofuran, diethyl ether or toluene at a temperature between about −78 to 20° C. Reactions of this type can be found in the chemistry literature; see, for example, Journal of Medicinal Chemistry 1986, 29, 1628-1637, Journal of Medicinal Chemistry 2008, 51, 7216-7233, Bioorganic & Medicinal Chemistry 2004, 12, 5465-5483 and Tetrahedron Letters 2006, 47, 817-820.

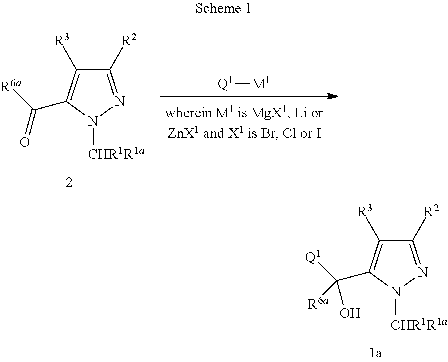

Alternatively, compounds of Formula 1a can be prepared as shown in Scheme 2. In Method A of Scheme 2, ketones of Formula 3 are reacted with an organometallic reagent of formula R^{6a}-M¹ using conditions analogous to Scheme 1 to provide compounds of Formula 1a wherein R^{6a} is alkyl. In Method B, compounds of Formula 3 are contacted with a hydride-containing reducing agent such as sodium borohydride, lithium aluminum hydride or diisobutylaluminum hydride in a solvent such as methanol, ethanol, tetrahydrofuran or diethyl ether at a temperature between about −20 to 20° C. to provide compounds of Formula 1a wherein R^{6a} is H. Other reduction methods known in the art may also be employed, such as catalytic hydrogenation as shown in Method C of Scheme 2. Typical reaction conditions involve exposing a compound of Formula 3 to hydrogen gas at a pressure of about 100 to 500 kPa, in the presence of a metal catalyst such as palladium or ruthenium supported on an inert carrier such as activated carbon, in a solvent such as ethanol at about 20° C. This type of reduction is well-known; see, for example, Catalytic Hydrogenation, L. Cerveny, Ed., Elsevier Science, Amsterdam, 1986, Organometallics 2010, 29(3), 554-561 and Organic Letters 2003, 5(26), 5039-5042. One skilled in the art will recognize that certain other functionalities that may be present in compounds of Formula 3 can also be reduced under catalytic hydrogenation conditions, thus requiring a suitable choice of catalyst and reaction conditions. In some cases the presence of a chiral diamine ligand having at least one N—H bond results in higher chemoselectivity of the desired compound (i.e. the carbonyl moiety is selectively reduced over certain other functionalities that may be present in compounds of Formula 3).

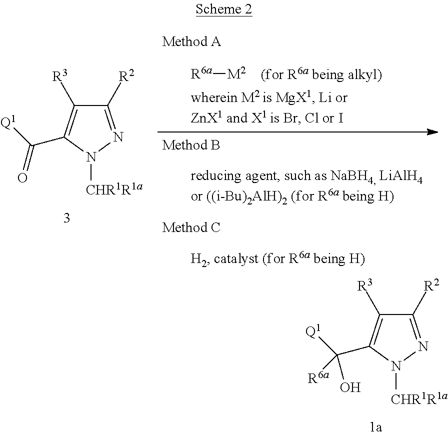

As is shown in Scheme 3, intermediates of Formula 2 wherein R^{6a} is alkyl can be prepared by contacting an organometallic reagent of formula R^{6a}-M² with an amide of Formula 4 (e.g., Weinreb amides). In this method compounds of formula R^{6a}-M² are Grignard reagents (i.e. M² is MgX² and X² is Br or Cl, for example, methylmagnesium chloride or bromide) or organolithium reagents (i.e. M² is Li, for example, methyllithium or tert-butyllithium). The reaction is typically conducted in a suitable solvent such as diethyl ether, tetrahydrofuran or toluene at a temperature between about −78 to 20° C. Compounds of Formula 2 can be isolated by quenching the reaction mixture with aqueous acid, extracting with an organic solvent and concentrating. Intermediates of Formula 2 wherein R^{6a} is H can be prepared by treating compounds of Formula 4 with a metal hydride reducing agent such as lithium aluminum hydride or diisobutylaluminum hydride. For conditions see Bioorganic & Medicinal Chemistry Letters 2013, 23, 6467-6473.

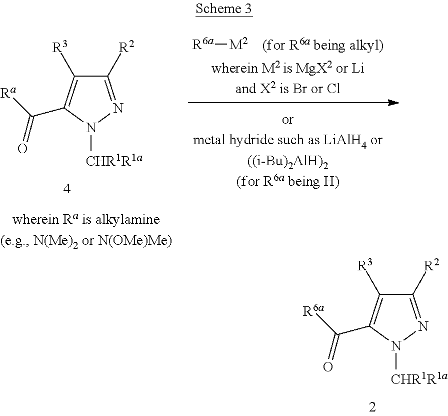

Amides of Formula 4 can be prepared by methods known in the art. For example, as shown in Scheme 4, compounds of Formula 4 wherein R^{a} is N(OMe)Me can be synthesized by conversion of a carboxylic acid of Formula 5 to the corresponding acid chloride, which is subsequently treated with N-methoxymethanamine to provide compounds of Formula 4 wherein R^{a} is N(OMe)Me. Reactions of this type are well-known in the chemistry literature; see, for example, publications relating to Weinreb amide preparation. For specific conditions see Bioorganic & Medicinal Chemistry Letters 2013, 23, 6467-6473 and Tetrahedron Letters 1981, 22(39), 3815-3818.

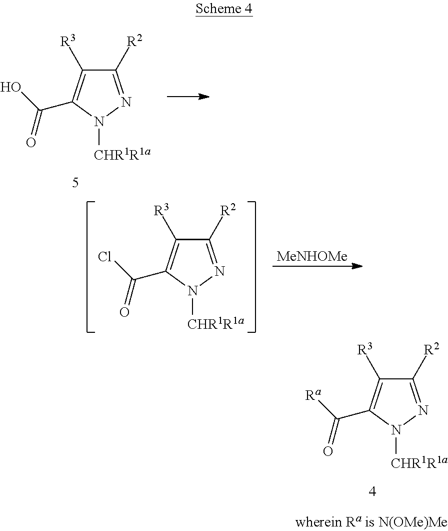

As shown in Scheme 5, carboxylic acids of Formula 5 can be prepared from the corresponding esters of Formula 6 using a variety of methods reported in the chemical literature, including nucleophilic cleavage under anhydrous conditions or hydrolysis involving the use of either acids or bases (see T. W. Greene and P. G. M. Wuts, Protective Groups in Organic Synthesis, 2nd ed., John Wiley & Sons, Inc., New York, 1991, pp. 224-269 for a review of methods). Base-catalyzed hydrolytic methods are preferred to prepare the carboxylic acids of Formula 5 from the corresponding esters. Suitable bases include alkali metal such as lithium, sodium or potassium hydroxide. For example, the esters can be dissolved in an alcohol such as methanol or a mixture of water and methanol. Upon treatment with sodium hydroxide or potassium hydroxide, the ester saponifies to provide the sodium or potassium salt of the carboxylic acid. Acidification with a strong acid, such as hydrochloric acid or sulfuric acid, gives the carboxylic acid.

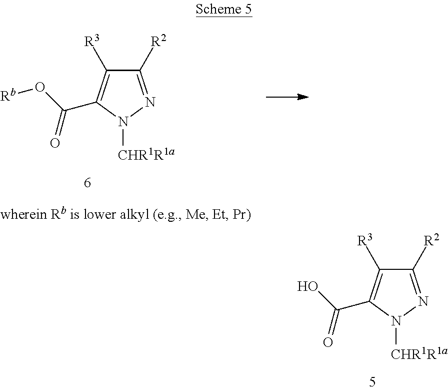

As shown in Scheme 6, compounds of Formula 7 can be converted to pyrazoles of Formula 6 through condensation with hydrazines of formula NH₂NH—CHR¹R^{1a}, which are commercially available or can be prepared by methods known in the art. The reaction is conducted in a suitable solvent such as ethanol, methanol or acetonitrile, at a temperature between about ambient temperature to the reflux temperature of the solvent. Two regioisomers can result from these types of reactions, which can be separated by standard techniques such as column chromatography. For a relevant reference, see for example, PCT Patent Publication WO 2009/013211.

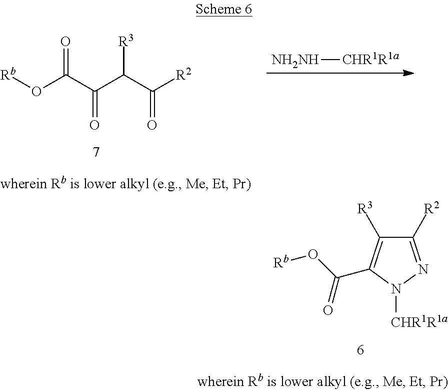

Compounds of Formula 7 can be prepared by acylation of a compound of Formula 8 with an oxalate derivative of Formula 9. The reaction is typically run in the presence of a base such as sodium ethoxide or lithium bis(trimethylsilyl)amide at a temperature between about −78 to 50° C. in solvents like ethanol, or with lithium hexamethyldisilazide at a temperature between about −78° C. to ambient temperature in solvents like ether.

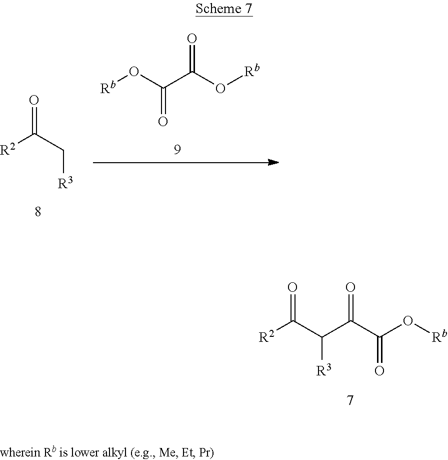

Intermediates of Formula 3 (shown Scheme 2) can be prepared using a method analogous to Scheme 3, where an aryl organometallic reagent of formula Q¹-M² is reacted with a compound of Formula 4 to provide a compound of Formula 3, as shown in Scheme 9.

For a related reference, see Journal of Medicinal Chemistry 2009, 52, 3377-3384.

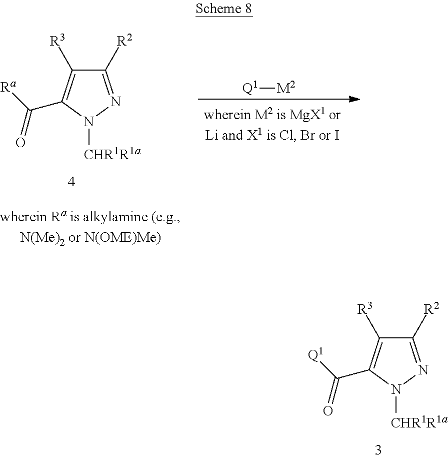

Alternatively, as shown in Scheme 9, compounds of Formula 3 can be prepared by reaction of an acid chloride of Formula 10 with a compound of formula Q¹-H using Friedel-Crafts condensation techniques. Typically the reaction is run in the presence of a Lewis acid (such as aluminum chloride or tin tetrachloride) and a solvent such as dichloromethane, 1,2-dichloroethane, tetrachloroethane, benzene or 1,2-dichlorobenzene, at a temperature between about −10 to 220° C. Friedel-Crafts reactions are documented in a variety of published references including Canadian Journal of Chemistry 1986, 64(11) 2211-2219, Journal of Heterocyclic Chemistry 2010, 47(5) 1040-1048 and J. March, Advanced Organic Chemistry, McGraw-Hill, New York, p 490 and references cited within.

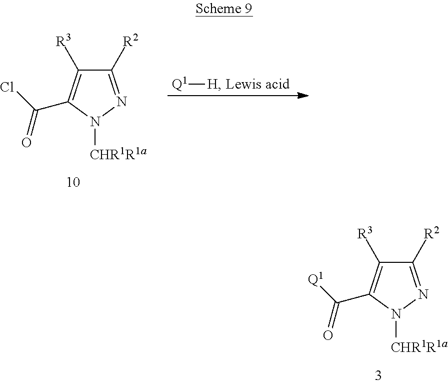

As shown in Scheme 11, Compounds of Formula 1 wherein X is O, S or NR⁵ can be prepared by reacting compounds of Formula 10 (e.g., 5-hydroxypyrazoles for X being 0, 5-mercaptopyrazoles for X being S or 5-aminopyrazoles for X being NR⁵) with compounds of formula Q¹-L¹ wherein L¹ is a leaving group such as halogen (e.g., Cl, Br or I) or (halo)alkylsulfonate (e.g., p-toluenesulfonate, methanesulfonate or trifluoromethanesulfonate) optionally in the presence of a metal catalyst, and generally in the presence of a base and a polar aprotic solvent such as N,N-dimethylformamide or dimethyl sulfoxide. For compounds of formula Q¹-L¹ wherein Q¹ is attached through a spa-hybridized carbon atom, L¹ is typically Cl, Br, I or a sulfonate (e.g., methanesulfonate). For Compounds of formula Q¹-L¹ wherein Q¹ is an aromatic ring lacking an electron-withdrawing substituent(s), or in general, to improve reaction rate, yield or product purity, the use of a metal catalyst (e.g., metal or metal salt) in amounts ranging from catalytic up to superstoichiometric can facilitate the desired reaction. Typically for these conditions, L¹ is Br, I or a sulfonate such as methyl trifluoromethanesulfonate or —OS(O)₂(CF₂)₃CF₃. For example, the reaction can be run in the presence of a metal catalyst such as copper salt complexes (e.g., CuI with N,N′-dimethylethylenediamine, proline or bipyridyl), palladium complexes (e.g., tris(dibenzylideneacetone)dipalladium(0)) or palladium salts (e.g., palladium acetate) with ligands such as 4,5-bis(diphenylphosphino)-9,9-dimethylxanthene, 2-dicyclohexylphosphino-2′,4′,6′-triisopropylbiphenyl or 2,2′-bis(diphenylphosphino)1,1′-bi-naphthalene, with a base such as potassium carbonate, cesium carbonate, potassium phosphate, sodium phenoxide or sodium tert-butoxide and a solvent such as N,N-dimethylformamide, 1,2-dimethoxyethane, dimethyl sulfoxide, 1,4-dioxane or toluene, optionally containing an alcohol such as ethanol. For relevant references, see PCT Patent Publication WO 2012/030922 (see Example 1, Step C and Example 2, Step G) and Archives of Pharmacal Research 2002, 25(6), 781-785. Also, the method of Scheme 11 is illustrated in present Example 1, Step B and Example 3, Step B.

One skilled in the art will appreciate that the leaving group L¹ attached to compounds of formula Q¹-L¹ should be selected in view of the relative reactivity of other functional groups present on formula Q¹-L¹ (i.e. substituents attached to Q¹), so that the group L¹ is displaced and not the functional groups to give the final desired compounds of Formula 1.

General methods useful for preparing starting compounds of Formula 11 are well-known in the art; see, for example, Journal für Praktische Chemie (Liepzig) 1911, 83, 171-182, Journal of the American Chemical Society 1954, 76, 501-503 and PCT Patent Publication WO 2012/030922 (see Example 1, Steps A-B and Example 2, Steps A-F). Also, present Example 1, Step A and Example 3, Step A illustrate the preparation of compounds of Formula 11.

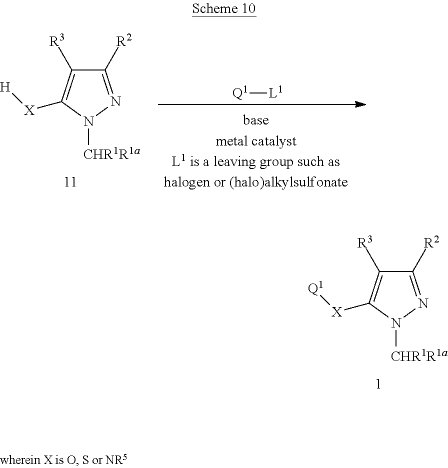

As illustrated in Scheme 11, compounds of Formula 1 wherein X is O, S or NR⁵ can also be prepared by reacting a compound of Formula 12 wherein L¹ is a leaving group such as halogen (e.g., Cl, Br or I) or (halo)alkylsulfonate (e.g., p-toluenesulfonate, methanesulfonate or trifluoromethanesulfonate) with a compound formula Q¹X—H under conditions analogous to those described for Scheme 10. For references illustrating this method see, for example, Synthesis 2012, 44, 2058-2061 and Organic Letters 2014, 16, 832-835.

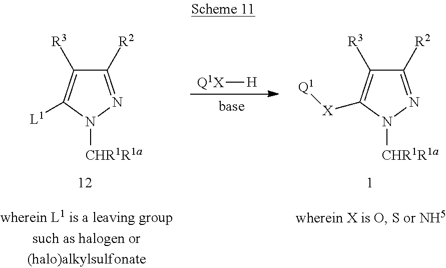

As shown in Scheme 12, intermediates of Formula 12 wherein L¹ is Br, Cl or I can be prepared from compounds of Formula 11 wherein X is NH using typical Sandmeyer reaction conditions. For example, addition of tert-butyl nitrite to a solution of a 5-aminopyrazole of Formula 11 in the presence of CuBr₂ in a solvent such as acetonitrile provides the corresponding 5-bromopyrazole of Formula 12. For a related reference, see Bioorganic & Medicinal Chemistry Letters 2013, 23, 6569-6576.

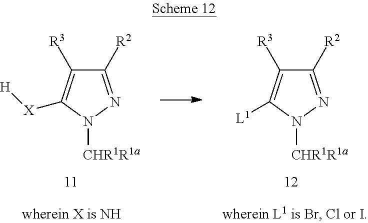

As shown in Scheme 13, compounds of Formula 12 wherein L¹ is fluoroalkylsulfonyl can be prepared from compounds of Formula 11 wherein X is O using the method described in Synlett 2004, (5), 795-798.

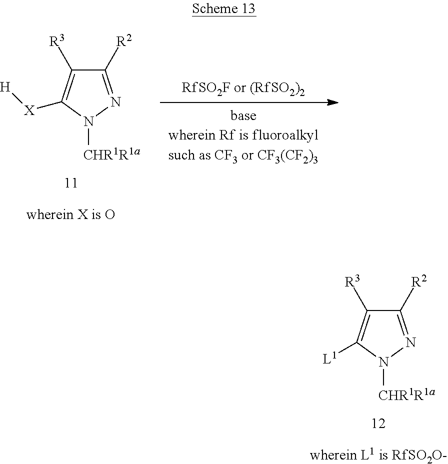

As shown in Scheme 14, compounds of Formula 1 can also be prepared by reacting a compound of Formula 13 with an alkylating agent of formula L¹-CHR¹R^{1a} wherein L¹ is a leaving group such as halogen (e.g., Cl, Br or I) or (halo)alkylsulfonate (e.g., p-toluenesulfonate, methanesulfonate or trifluoromethanesulfonate), preferably in the presence of a base such as 1,8-diazabicyclo[5.4.0]undec-7-ene, potassium carbonate or potassium hydroxide, and in a solvent such as N,N-dimethylformamide, tetrahydrofuran, toluene or water. General procedures for alkylations of this type are well-known in the art and can be readily adapted to prepare compounds of the present invention. Particularly useful alkylating agents for preparing compounds of Formula 1 wherein R¹ and R^{1a} are H are diazomethane or iodomethane using general procedures known in the art, such as those described in Journal of Heterocyclic Chemistry 2004, 41, 931-939, Chem. Pharm. Bull. 1984, 32(11), 4402-4409 and PCT Patent Publication WO 2012/030922 (see Example 9, Step B). Compounds of Formula 1 wherein R¹ and R^{1a} form an optionally substituted cyclopropyl ring can likewise be prepared by reaction of a compound of Formula 13 with an organometallic reagent, such as tricyclopropylbismuth, in the presence of a catalyst, such as copper acetate, under conditions known in the art such as those described in J. Am. Chem. Soc. 2007, 129(1), 44-45.

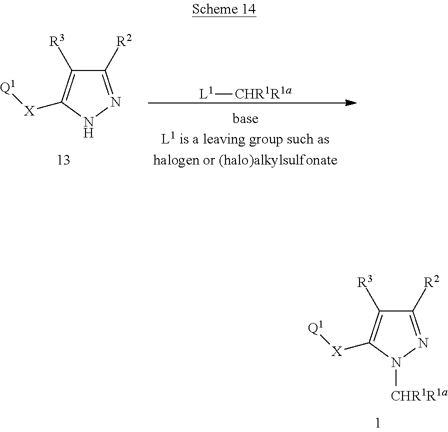

Compounds of Formula 13 are known and can be prepared by a variety of methods disclosed in the chemical literature. For example, as shown in Scheme 15, compounds of Formula 15 can first be prepared by contacting a compound of Formula 14 with hydrazine hydrochloride. The reaction can be run in a variety of solvents, but optimal yields are typically obtained when the reaction is run in ethanol at a temperature between about ambient temperature and the reflux temperature of the solvent. General procedures for this type of reaction are well documented in the chemical literature; see, for example, Journal of Medicinal Chemistry 2006, 49, 4762-4766 and PCT Patent Publication WO 2009/137651 (see Example 39, Step C). In a subsequent step, compounds of Formula 15 are halogenated or alkylated to provide compounds of Formula 13 wherein R² is halogen or alkyl. Halogenation can be achieved using a variety of halogenating agents known in the art such as elemental halogen (e.g., Cl₂, Br₂, I₂), sulfuryl chloride, iodine monochloride or a N-halosuccinimide (e.g., NBS, NCS, NIS) in an appropriate solvent such as N,N-dimethylformamide, carbon tetrachloride, acetonitrile, dichloromethane or acetic acid. Alkylation is achieved by contacting a compound of Formula 15 with a metalating agent, followed by an alkylating agent of formula R²-L¹ (wherein L¹ is a leaving group such as Cl, Br, I or a sulfonate, for example, p-toluenesulfonate, methanesulfonate or trifluoromethanesulfonate). Suitable metalating agents include, for example, n-butyllithium (n-BuLi), lithium diisopropylamide (LDA) or sodium hydride (NaH). As used herein, the terms “alkylation” and “alkylating agent” are not limited to R² being an alkyl group, and include in addition to alkyl groups such as alkylthio, haloalkyl, alkenyl, haloalkenyl, alkynyl, and the like. For reaction conditions see, Synthetic Communications 2008, 38(5), 674-683 and PCT Patent Publication WO 2009/137651 (see Example 39, Step D).

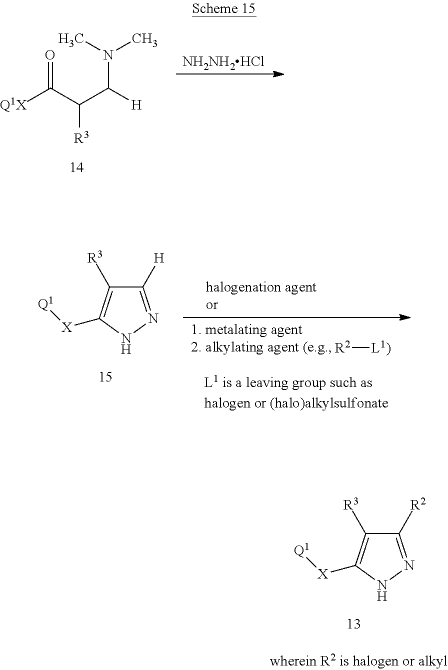

As shown in Scheme 16, compounds of Formula 14 can be prepared from ketones of Formula 16 and N,N-dimethylformamide dimethyl acetal using the method described in Journal of Medicinal Chemistry 2006, 49, 4762-4766. The reaction is typically conducted in a solvent such as benzene, toluene or xylenes at a temperature between about ambient temperature and the reflux temperature of the solvent.

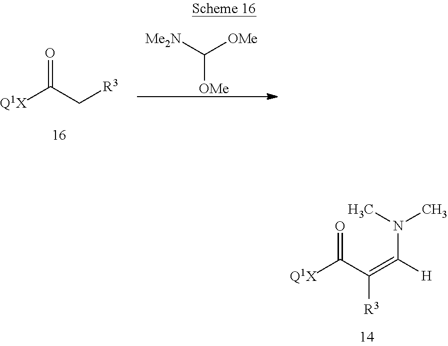

As shown in Scheme 17, ketones of Formula 16 can be prepared by contacting a compound of Formula 17 with a compound of formula Q¹X—H using the method described in Journal of Medicinal Chemistry 2006, 49, 4762-4766.

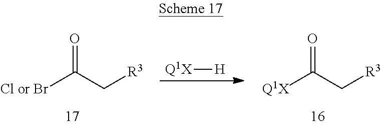

Compounds of Formula 1 can also be prepared as shown in Scheme 18. In this method a compound of Formula 18 is first treated with an organometallic agent of formula Rb-M³ such an alkyl lithium base (e.g., n-butyllithium, s-butyllithium or lithium diisopropylamide) or a Grignard reagent in a solvent such as toluene, diethyl ether, tetrahydrofuran or dimethoxymethane at temperatures ranging from about −78° C. to ambient temperature. Anions of Formula 18a are then contacted with an electrophile of Formula 19 or 20. The use and choice of an appropriate electrophile of Formula 19 or 20 will depend on the desired compound of Formula 1 and will be apparent to one skilled in chemical synthesis. For example, aldehydes of the formula Q¹CHO provide compounds Formula 1 wherein X is CH(OH) and chlorosulfides of formula Q¹SCl or disulfides formula of Q¹S—S-Q¹ provide compounds Formula 1 wherein X is S. There are a wide-variety of general methods described in the synthetic literature for metalation/alkylation reactions which can be readily adapted to prepare compounds of the present invention; see, for example, J. Org. Chem. 2010, 75, 984-987.

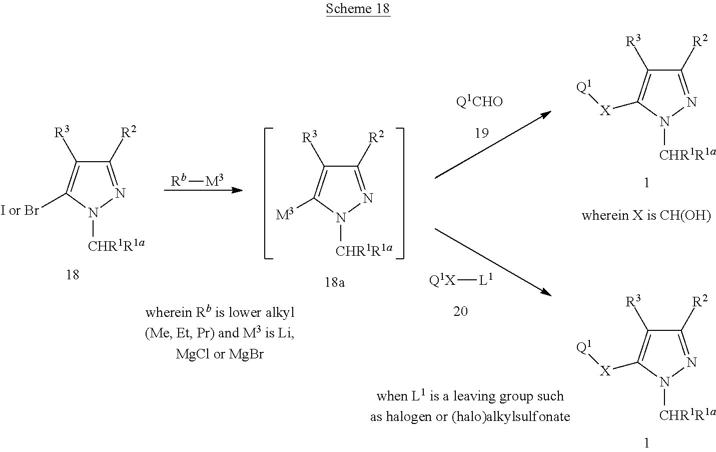

Electrophiles of Formulae 19 and 20 are commercially available and can be prepared by methods known in the art. Compounds of Formula 18 can be prepared by methods analogous to those disclosed in Schemes 12 and 14 and by a variety of methods disclosed in the chemical literature.

Compounds of Formula 1 can be subjected to various nucleophilic and metalation reactions to add substituents or modify existing substituents, and thus provide other functionalized compounds of Formula 1. For example, as shown in Scheme 19, compounds of Formula 1b (i.e. Formula 1 wherein X in NR⁵ and R⁵ is other than H) can be prepared by reacting corresponding compounds of Formula 1c (i.e. Formula 1 wherein X is NR⁵ and R⁵ is H) with an electrophile comprising R⁵ (i.e. Formula 21) typically in the presence of a base such as NaH and a polar solvent such as N,N-dimethylformamide. In this context the expression “electrophile comprising R⁵” means a chemical compound capable of transferring an R⁵ moiety to a nucleophile (such as the nitrogen atom attached to Q¹ in Formula 1b). Often electrophiles comprising R⁵ have the formula R⁵L² wherein L² is a nucleofuge (i.e. leaving group in nucleophilic reactions). Typical nucleofuges include halogens (e.g., Cl, Br, I) and sulfonates (e.g., OS(O)₂CH₃, OS(O)₂CF₃, OS(O)₂-(4-CH₃-Ph)).

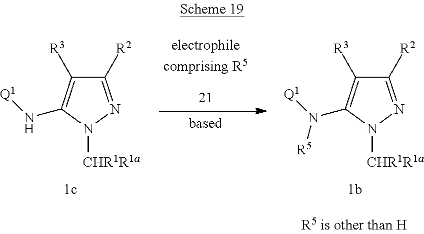

As shown in Scheme 20, a fluorine can be introduced at the 3-position of the pyrazole ring by treating compounds Formula 1d (i.e. Formula 1 wherein R² is chlorine) with potassium fluoride or cesium fluoride in the presence of a solvent such as dimethyl sulfoxide or N,N-dimethylformamide at 0-25° C. for 30 minutes to 4 h, using procedures described in Zhurnal Organicheskoi Khimii 1983, 19, 2164-2173.

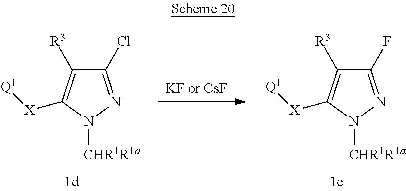

As shown in Scheme 21, sulfoxides and sulfones of Formula 1f (i.e. Formula 1 wherein X is S(═O)ₘ and m is 1 or 2) can be prepared by oxidation of compounds of Formula 1g (i.e. Formula 1 wherein X is S). Typically an oxidizing agent in an amount from about 1 to 4 equivalents, depending on the oxidation state of the desired product, is added to a mixture of a compound of Formula 1g and a solvent. Useful oxidizing agents include Oxone® (potassium peroxymonosulfate), potassium permanganate, hydrogen peroxide, sodium periodate, peracetic acid and 3-chloroperbenzoic acid. The solvent is selected with regard to the oxidizing agent employed. Aqueous ethanol or aqueous acetone is preferably used with potassium peroxymonosulfate, and dichloromethane is generally preferable with 3-chloroperbenzoic acid. Useful reaction temperatures typically range from about −78 to 90° C. Oxidation reactions of this type are described in J. Agric. Food Chem. 1984, 32, 221-226 and J. Agric. Food Chem. 2008, 56, 10160-10167.

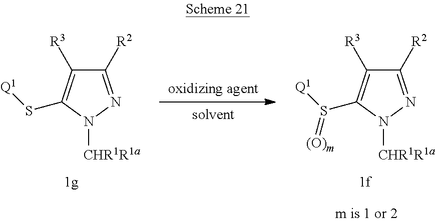

It is recognized by one skilled in the art that various functional groups can be converted into others to provide different compounds of Formula 1. For example, compounds of Formula 1 wherein R² is methyl, ethyl, cyclopropyl, and the like, can be modified by free-radical halogenation to form compounds of Formula 1 wherein R² is halomethyl, haloethyl, halocyclopropyl, and the like. Compounds of Formula 1 wherein R² is halomethyl can be used to prepare compounds of Formula 1 wherein R² is hydroxymethyl or cyanomethyl. Compounds of Formula 1, or intermediates for their preparation, may contain aromatic nitro groups, which can be reduced to amino groups, and then converted via reactions well-known in the art (e.g., Sandmeyer reaction) to various halides. By similar known reactions, aromatic amines (anilines) can be converted via diazonium salts to phenols, which can then be alkylated to prepare compounds of Formula 1 with alkoxy substituents. Likewise, aromatic halides such as bromides or iodides prepared via the Sandmeyer reaction can react with alcohols under copper-catalyzed conditions, such as the Ullmann reaction or known modifications thereof, to provide compounds of Formula 1 that contain alkoxy substituents. Additionally, some halogen groups, such as fluorine or chlorine, can be displaced with alcohols under basic conditions to provide compounds of Formula 1 containing the corresponding alkoxy substituents. Compounds of Formula 1 or precursors thereof in which R² is halide, preferably bromide or iodide, are particularly useful intermediates for transition metal-catalyzed cross-coupling reactions to prepare compounds of Formula 1. These types of reactions are well documented in the literature; see, for example, Tsuji in Transition Metal Reagents and Catalysts: Innovations in Organic Synthesis, John Wiley and Sons, Chichester, 2002; Tsuji in Palladium in Organic Synthesis, Springer, 2005; and Miyaura and Buchwald in Cross Coupling Reactions: A Practical Guide, 2002; and references cited therein.

It is recognized that some reagents and reaction conditions described above for preparing compounds of Formula 1 may not be compatible with certain functionalities present in the intermediates. In these instances, the incorporation of protection/deprotection sequences or functional group interconversions may be necessary to obtaining the desired products. The use and choice of the protecting groups will be apparent to one skilled in chemical synthesis (see, for example, Greene, T. W.; Wuts, P. G. M. Protective Groups in Organic Synthesis, 2nd ed.; Wiley: New York, 1991). One skilled in the art will recognize that, in some cases, after the introduction of a given reagent as it is depicted in any individual scheme above, it may be necessary to perform additional routine synthetic steps not described in detail to complete the synthesis of compounds of Formula 1. One skilled in the art will also recognize that it may be necessary to perform a combination of the steps illustrated in the above schemes in an order other than that which is implied by the particular sequence presented to prepare the compounds of Formula 1. One skilled in the art will also recognize that compounds of Formula 1 and the intermediates described herein can be subjected to various electrophilic, nucleophilic, radical, organometallic, oxidation, and reduction reactions to add substituents or modify existing substituents.

Without further elaboration, it is believed that one skilled in the art using the preceding description can utilize the present invention to its fullest extent. The following Examples are, therefore, to be construed as merely illustrative, and not limiting of the disclosure in any way whatsoever. Steps in the following Examples illustrate a procedure for each step in an overall synthetic transformation, and the starting material for each step may not have necessarily been prepared by a particular preparative run whose procedure is described in other Examples or Steps. Percentages are by weight except for chromatographic solvent mixtures or where otherwise indicated. Parts and percentages for chromatographic solvent mixtures are by volume unless otherwise indicated. ¹H NMR spectra are reported in ppm downfield from tetramethylsilane; “s” means singlet, “d” means doublet, “t” means triplet, “m” means multiplet, and “dd” means doublet of doublets.

### Example 1

### Preparation of N-(4-chloro-2,6-difluorophenyl)-4-(1-cyclohexen-1-yl)-1,3-dimethyl-1H-pyrazol-5-amine (Compound 6)

### Step A: Preparation of 4-(1-cyclohexen-1-yl)-1,3-dimethyl-1H-pyrazol-5-amine

To a mixture of 1,3-dimethyl-1H-pyrazol-5-amine (5.0 g, 45 mmol) in acetic acid (50 mL) was added cyclohexanone (9.4 mL, 91 mmol). The reaction mixture was stirred at ambient temperature for 45 h, and then concentrated under reduced pressure. The resulting material was diluted with water (1 L), and then made basic by the addition of sodium hydroxide solution (10% in water). The resulting solid precipitate was collected by filtration and recrystallized from ethyl acetate to provide the title compound as a solid (7.0 g).

¹H NMR (DMSO-d₆): δ 5.41 (m, 1H), 4.67 (s, 2H), 3.42 (s, 3H), 2.11 (m, 4H), 1.94 (s, 3H), 1.60 (m, 4H).

M.P.: 174-176° C.

### Step B: Preparation of N-(4-chloro-2,6-difluorophenyl)-4-(1-cyclohexen-1-yl)-1,3-dimethyl-1H-pyrazol-5-amine

To a mixture of 4-(1-cyclohexen-1-yl)-1,3-dimethyl-1H-pyrazol-5-amine (i.e. the product of Step A) (3.0 g, 16 mmol) in toluene (30 mL) was added 2-bromo-5-chloro-1,3-difluorobenzene (2.3 mL, 19 mmol), 4,5-bis(diphenylphosphino)-9,9-dimethylxanthene (also known as xantphos) (1.8 g, 3.1 mmol), potassium phosphate (6.7 g, 32 mmol) and tris(dibenzylideneacetone)dipalladium (1.4 g, 1.5 mmol) while purging with argon gas. The reaction mixture was heated at reflux for 5 h and then diluted with ethyl acetate (100 mL). The resulting mixture was washed with water (30 mL) and saturated sodium chloride solution (30 mL), and then dried over sodium sulfate, filtered and concentrated under reduced pressure. The resulting material was purified by silica gel column chromatography (40% ethyl acetate in hexanes as eluant) to provide the title compound, a compound of the present invention, as a yellow solid (800 mg).

¹H NMR (DMSO-d₆): δ 7.57 (s, 1H), 7.22 (m, 2H), 5.38 (m, 1H), 3.58 (s, 3H), 2.01 (s, 3H), 1.83 (m, 4H), 1.32 (m, 4H).

M.P.: 142-145° C.

### Example 2

### Preparation of N-(4-chloro-2,6-difluorophenyl)-4-cyclohexyl-1,3-dimethyl-1H-pyrazol-5-amine (Compound 46)

To a mixture of N-(4-chloro-2,6-difluorophenyl)-4-(1-cyclohexen-1-yl)-1,3-dimethyl-1H-pyrazol-5-amine (i.e. the product of Step B, Example 1) (1.8 g, 5.3 mmol) in ethanol (20 mL) was added palladium on carbon (10%, 0.57 g, 0.53 mmol). The reaction mixture was stirred under a hydrogen balloon for 16 h at ambient temperature. The reaction mixture was filtered to remove the palladium on carbon and the filtrate was concentrated under reduced pressure. The resulting material was purified by HPLC to provide the title compound, a compound of the present invention, as a white solid (200 mg).

¹H NMR (DMSO-d₆): δ 7.48 (t, 1H), 7.20 (m, 2H), 3.48 (s, 3H), 2.21 (m, 1H), 2.09 (s, 3H), 1.62 (m, 3H), 1.50 (d, 2H), 1.38 (m, 2H), 1.08 (m, 3H).

M.P.: 128-132° C.

### Example 3

### Preparation of N-(4-chloro-2, 6-difluorophenyl)-1,3-dimethyl-4-(1-methyl-1-propen-1-yl)-1H-pyrazol-5-amine (Compound 24)

### Step A: Preparation of 1,3-dimethyl-4-(1-methyl-1-propen-1-yl)-1H-pyrazol-5-amine

To a mixture of 1,3-dimethyl-1H-pyrazol-5-amine (10 g, 90 mmol) in acetic acid (100 mL) was added 2-butanone (16 mL, 180 mmol). The reaction mixture was stirred at ambient temperature for 45 h, and then concentrated under reduced pressure. The resulting material was diluted with water (1 L), and then made basic by the addition of sodium hydroxide (10% in water). The resulting precipitate was collected by filtration and recrystallized from ethyl acetate to provide the title compound as a solid (4.5 g).

¹H NMR (DMSO-d₆): δ 5.21 (m, 1H), 4.71 (s, 2H), 3.43 (s, 3H), 1.94 (s, 3H), 1.80 (m, 3H), 1.68 (dd, 3H).

M.P.: 120-123° C.

### Step B: Preparation of N-(4-chloro-2, 6-difluorophenyl)-1,3-dimethyl-4-(1-methyl-1-propen-1-yl)-1H-pyrazol-5-amine

To a mixture of 1,3-dimethyl-4-(1-methyl-1-propen-1-yl)-1H-pyrazol-5-amine (i.e. the product of Step A) (3.0 g, 18 mmol) in toluene (30 mL) was added 2-bromo-5-chloro-1,3-difluorobenzene (3.4 mL, 27 mmol), 4,5-bis(diphenylphosphino)-9,9-dimethylxanthene (also known as xantphos), (2.1 g, 3.6 mmol), potassium phosphate (7.7 g, 36 mmol) and tris(dibenzylideneacetone)dipalladium (1.6 g, 1.8 mmol) while purging with argon gas. The reaction mixture was heated at reflux for 16 h and then diluted with ethyl acetate (100 mL). The resulting mixture was washed with water (30 mL) and saturated sodium chloride solution (30 mL), and then dried over sodium sulfate, filtered and concentrated under reduced pressure. The resulting material was purified by silica gel column chromatography (40% ethyl acetate in hexanes as eluant) to provide the title compound, a compound of the present invention, as a white solid (1.1 g).

¹H NMR (DMSO-d₆): δ 7.63 (t, 1H), 7.20 (m, 2H), 5.24 (m, 1H), 3.60 (s, 3H), 1.92 (s, 3H), 1.60 (s, 3H), 1.31 (d, 3H).

M.P.: 140-142° C.

### Example 4

### Preparation of N-(4-chloro-2, 6-difluorophenyl)-1,3-dimethyl-4-(1-methylpropyl)-1H-pyrazol-5-amine (Compound 23)

To a mixture of N-(4-chloro-2, 6-difluorophenyl)-1,3-dimethyl-4-(1-methyl-1-propen-1-yl)-1H-pyrazol-5-amine (i.e. the product of Step B, Example 3) (0.60 g, 1.92 mmol) in ethanol (15 mL) was added palladium on carbon (10%, 0.10 g, 0.97 mmol). The reaction mixture was stirred for 4 h under a hydrogen atmosphere at 30 psi, and then for 32 h under a hydrogen atmosphere at 50 psi. The reaction mixture was filtered to remove the palladium on carbon and the filtrate was concentrated under reduced pressure to provide the title compound, a compound of the present invention, as a solid (130 mg).

¹H NMR (DMSO-d₆): δ 7.47 (t, 1H), 7.23 (d, 2H), 3.50 (s, 3H), 2.30 (m, 1H), 2.06 (s, 3H), 1.37 (m, 2H), 1.01 (d, 3H), 0.65 (t, 3H).

M.P.: 80-84° C.

### Example 5

### Preparation of 4-cyclohexyl-α-(2,4-difluorophenyl)-1,3-dimethyl-1H-pyrazole-5-methanol (Compound 12)

### Step A: Preparation of 4-(1-cyclohexen-1-yl)-1,3-dimethyl-1H-pyrazol-5-amine

To a mixture of 1,3-dimethyl-1H-pyrazol-5-amine (5.0 g, 45 mmol) in acetic acid (50 mL) was added cyclohexanone (9.4 mL, 91 mmol). The reaction mixture was stirred at ambient temperature for 45 h, and then concentrated under reduced pressure. The resulting material was diluted with water (1 L), and then made basic by the addition of sodium hydroxide solution (10% in water). The resulting solid precipitate was collected by filtration to provide the title compound as a yellow solid (7.0 g).

¹H NMR (CDCl₃): δ 5.58 (m, 1H), 3.61 (s, 3H), 3.45 (br s, 2H), 2.22-2.12 (m, 4H), 2.16 (s, 3H), 1.8-1.6 (m, 4H).

### Step B: Preparation of 4-cyclohexyl-1,3-dimethyl-1H-pyrazol-5-amine

To a mixture of 4-(1-cyclohexen-1-yl)-1,3-dimethyl-1H-pyrazol-5-amine (i.e. the product of Step A) (3 g, 16 mmol) in ethanol (30 mL) was added palladium on carbon (10%, 1 g, 0.94 mmol). The reaction mixture was stirred under a hydrogen balloon for 16 h at ambient temperature. The reaction mixture was filtered through a pad of Celite® (diatomaceous earth) on a sintered glass frit funnel, rinsing with ethanol. The filtrate was concentrated under reduced pressure to provide the title compound as a white solid (2.8 g).

¹H NMR (CDCl₃): δ 3.61 (s, 3H), 3.23 (br s, 2H), 2.28 (m, 1H), 2.18 (s, 3H), 1.85-1.7 (m, 5H), 1.6-1.45 (m, 2H), 1.4-1.2-(m, 3H).

### Step C: Preparation of 5-bromo-4-cyclohexyl-1,3-dimethyl-1H-pyrazole

To a mixture of 4-cyclohexyl-1,3-dimethyl-1H-pyrazol-5-amine (i.e. the product of Step B) (2.8 g, 14.5 mmol) in acetonitrile (30 mL) was added copper(II) bromide (3.56 g, 15.95 mmol). The reaction mixture was cooled in an ice bath, and then isoamyl nitrite (2.9 g, 24.66 mmol) was added. After 15 minutes, the reaction was slowly warmed to 50° C. and stirred for 2 h. The reaction mixture was allowed to cool to room temperature, and then aqueous hydrochloric acid (1 M, 10 mL) was added. The resulting mixture was extracted with ethyl acetate. The organic layer was washed with water and saturated sodium chloride solution, then dried over sodium sulfate and filtered. The filtrate was concentrated under reduced pressure to provide an oil. The oil was purified by silica gel chromatography (eluting with 20-40% ethyl acetate in hexanes) to provide the title compound as a green oil 1.4 g)

¹H NMR (CDCl₃): δ 3.8 (s, 3H), 2.45 (m, 1H), 2.25 (s, 3H) 1.9-1.6 (m, 7H), 1.45-1.2 (m, 3H).

### Step D: Preparation of 4-cyclohexyl-α-(2,4-difluorophenyl)-1,3-dimethyl-1H-pyrazole-5-methanol

To a mixture of 5-bromo-4-cyclohexyl-1,3-dimethyl-1H-pyrazole (i.e. the product of Step C) (500 mg, 1.95 mmol) in tetrahydrofuran (15 mL) at −78° C. was slowly added butyllithium (1.6 M, 1.34 mL, 2.18 mmol). The reaction mixture was stirred for 15 minutes, at −78° C., and then 2,4-difluorobenzaldehyde (305 mg, 2.18 mmol) was added. The reaction mixture was stirred at −78° C. for 1 h, and then aqueous saturated ammonium chloride solution (10 mL) was added. The resulting mixture was extracted with ethyl acetate. The combined organic layers were washed with water and saturated sodium chloride solution, dried over sodium sulfate and filtered. The filtrate was concentrated under reduced pressure to provide an oil. The oil was purified by silica gel chromatography (eluting with 20-40% ethyl acetate in hexanes) to provide the title compound, a compound of the present invention, as a white solid (112 mg).

¹H NMR (CDCl₃): δ 7.43 (m, 1H), 6.88 (m, 1H), 6.78 (m, 1H), 6.23 (m, 1H), 3.69 (s, 3H), 2.50 (m, 1H), 2.32 (br s, 1H), 2.28 (s, 3H) 1.8-1.45 (m, 7H), 1.35-1.15 (m, 3H).

By the procedures described herein together with methods known in the art, the compounds disclosed in the Tables that follow can be prepared. The following abbreviations are used in the Tables which follow: i means iso, c means cyclo, n means normal, s means secondary, Me means methyl, Bu means butyl, Pr means propyl, MeO means methoxy, CN means cyano, and Ph means phenyl.

TABLE 1

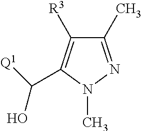

Q¹ is 2,4,6-tri-F—Ph
R³
CH₃
CH₃CH2
CH₃CH₂CH₂
(CH₃)₂CH
c-Pr
CH≡CCH₂—
CH₂═CH(CH₃)—
Bu
tert-Bu
(CH₃)₂CHCH₂
CH₃CH₂CH(CH₃)
c-Bu
CH₃CH═C(CH₃)—
CH≡CCH(CH₃)—
CH₂═CHCH(CH₃)—
pentyl
c-pentyl
CH₃C(CH₃)₂CH₂—
CH₃CH₂CH₂CH(CH₃)—
(CH₃CH₂)₂CH—
CH₃CH═C(Et)—
CH₂═CHCH(Et)—
CH₃CH₂CH═C(CH₃)—
CH₃CH═CHCH(CH₃)—
CH₃C≡CCH(CH₃)—
hexyl
c-hexyl
(CH₃)₂CHCH═C(Me)—
CH₃CH₂CH₂CH(Et)—
CH₃(CH₂)₃CH(CH₃)—
(CH₃)₂C═CHCH(CH₃)—
c-heptyl
c-octyl
CH₃OCH(CH₃)—
CH₃SCH(CH₃)—
CH₃OCH₂CH(CH₃)—
CH₃SCH₂CH(CH₃)—
CH₃CH₂OCH(CH₃)—
CH₃CH₂SCH(CH₃)—
CH₃O(CH₂)₂CH(CH₃)—
EtOCH₂CH(CH₃)—
CH₃CH₂CH₂OCH(Me)—
CF₃CH(CH₃)—
CF₃CH₂CH(CH₃)—
CH₃CH₂CH(CF₃)—
ClCH₂CH(CH₃)—

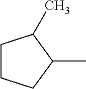

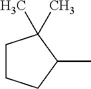

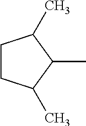

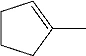

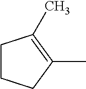

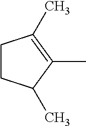

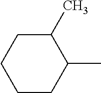

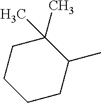

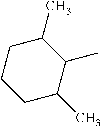

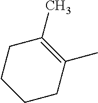

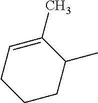

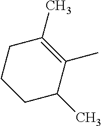

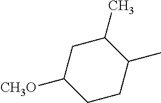

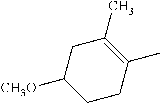

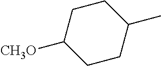

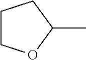

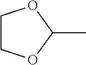

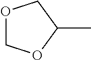

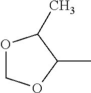

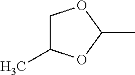

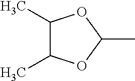

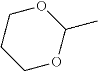

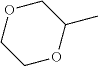

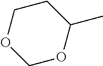

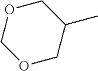

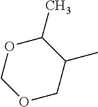

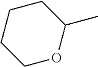

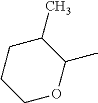

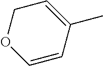

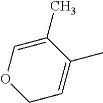

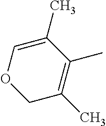

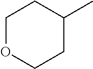

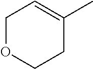

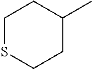

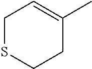

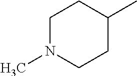

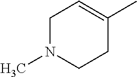

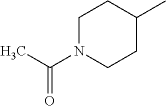

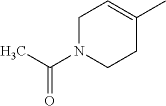

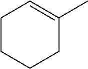

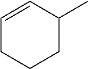

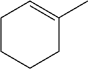

The present disclosure also includes Tables 1A through 28A, each of which is constructed the same as Table 1 above, except that the row heading in Table 1 (i.e. “Q¹ is 2,4,6-tri-F-Ph”) is replaced with the respective row headings shown below.

| Table | Row Heading |
|---|---|
| 1A | Q¹ is 2,6-di-F—Ph. |
| 2A | Q¹ is 2,6-di-F-4-MeO—Ph. |
| 3A | Q¹ is 2,6-di-F-4-Me—Ph. |
| 4A | Q¹ is 2,6-di-F-4-CN—Ph. |
| 5A | Q¹ is 2,6-di-F-4-Cl—Ph. |
| 6A | Q¹ is 2,6-di-F-4-Br—Ph. |
| 7A | Q¹ is 2,4-di-F—Ph. |
| 8A | Q¹ is 2,4-di-F-6-Cl—Ph. |
| 9A | Q¹ is 2,4-di-F-6-Br—Ph. |
| 10A | Q¹ is 2-Cl-6-F—Ph. |
| 11A | Q¹ is 2-Br-6-F—Ph. |
| 12A | Q¹ is 2-Cl-4-Me-6-F—Ph. |
| 13A | Q¹ is 2-Cl-4-MeO-6-F—Ph. |
| 14A | Q¹ is 2-Br-4-Me-6-F—Ph. |
| 15A | Q¹ is 2-Br-4-MeO-6-F—Ph. |
| 16A | Q¹ is 2,6-di-Cl-4-Me—Ph. |
| 17A | Q¹ is 2,6-di-Br-4-Me—Ph. |
| 18A | Q¹ is 2,4,6-tri-Cl—Ph. |
| 19A | Q¹ is 2-Cl-4-F—Ph. |
| 20A | Q¹ is 2-Cl-4-Me—Ph. |
| 21A | Q¹ is 2-Cl-4-MeO—Ph. |
| 22A | Q¹ is 2-Br-4-F—Ph. |
| 23A | Q¹ is 2-Br-4-Me—Ph. |
| 24A | Q¹ is 2-Br-4-MeO—Ph. |
| 25A | Q¹ is 2,4-di-Cl—Ph. |
| 26A | Q¹ is 2,6-di-Cl—Ph. |
| 27A | Q¹ is 2,4-di-Me—Ph. |
| 28A | Q¹ is 2,6-di-Me—Ph. |

TABLE 2

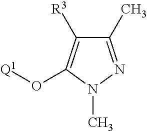

Q¹ is 2,4,6-tri-F—Ph
R³
CH₃
CH₃CH2
CH₃CH₂CH₂
(CH₃)₂CH
c-Pr
CH≡CCH₂—
CH₂═CH(CH₃)—
Bu
tert-Bu
(CH₃)₂CHCH₂
CH₃CH₂CH(CH₃)
c-Bu
CH₃CH═C(CH₃)—
CH≡CCH(CH₃)—
CH₂═CHCH(CH₃)—
pentyl
c-pentyl
CH₃C(CH₃)₂CH₂—
CH₃CH₂CH₂CH(CH₃)—
(CH₃CH₂)₂CH—
CH₃CH═C(Et)—
CH₂═CHCH(Et)—
CH₃CH₂CH═C(CH₃)—
CH₃CH═CHCH(CH₃)—
CH₃C≡CCH(CH₃)—
hexyl
c-hexyl
(CH₃)₂CHCH═C(Me)—
CH₃CH₂CH₂CH(Et)—
CH₃(CH₂)₃CH(CH₃)—
(CH₃)₂C═CHCH(CH₃)—
c-heptyl
c-octyl
CH₃OCH(CH₃)—
CH₃SCH(CH₃)—
CH₃OCH₂CH(CH₃)—
CH₃SCH₂CH(CH₃)—
CH₃CH₂OCH(CH₃)—
CH₃CH₂SCH(CH₃)—
CH₃O(CH₂)₂CH(CH₃)—
EtOCH₂CH(CH₃)—
CH₃CH₂CH₂OCH(Me)—
CF₃CH(CH₃)—
CF₃CH₂CH(CH₃)—
CH₃CH₂CH(CF₃)—
ClCH₂CH(CH₃)—

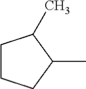

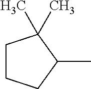

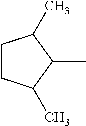

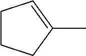

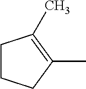

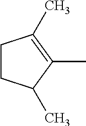

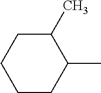

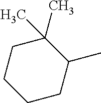

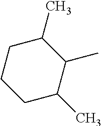

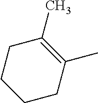

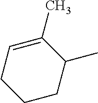

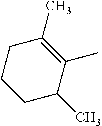

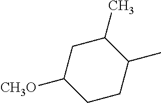

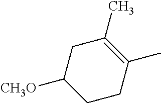

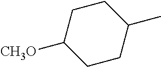

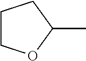

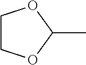

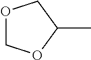

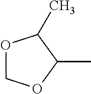

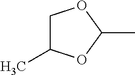

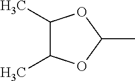

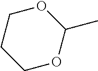

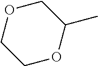

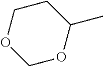

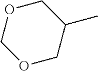

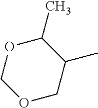

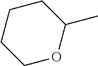

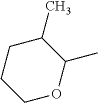

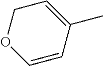

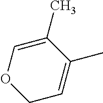

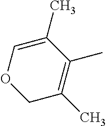

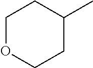

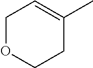

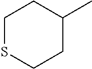

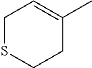

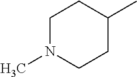

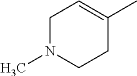

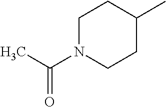

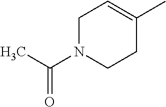

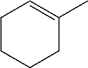

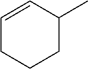

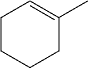

The present disclosure also includes Tables 1B through 28B, each of which is constructed the same as Table 2 above, except that the row heading in Table 2 (i.e. “Q¹ is 2,4,6-tri-F-Ph”) is replaced with the respective row headings shown below.

| Table | Row Heading |
|---|---|
| 1B | Q¹ is 2,6-di-F—Ph. |
| 2B | Q¹ is 2,6-di-F-4-MeO—Ph. |
| 3B | Q¹ is 2,6-di-F-4-Me—Ph. |
| 4B | Q¹ is 2,6-di-F-4-CN—Ph. |
| 5B | Q¹ is 2,6-di-F-4-Cl—Ph. |
| 6B | Q¹ is 2,6-di-F-4-Br—Ph. |
| 7B | Q¹ is 2,4-di-F—Ph. |
| 8B | Q¹ is 2,4-di-F-6-Cl—Ph. |
| 9B | Q¹ is 2,4-di-F-6-Br—Ph. |
| 10B | Q¹ is 2-Cl-6-F—Ph. |
| 11B | Q¹ is 2-Br-6-F—Ph. |
| 12B | Q¹ is 2-Cl-4-Me-6-F—Ph. |
| 13B | Q¹ is 2-Cl-4-MeO-6-F—Ph. |
| 14B | Q¹ is 2-Br-4-Me-6-F—Ph. |
| 15B | Q¹ is 2-Br-4-MeO-6-F—Ph. |
| 16B | Q¹ is 2,6-di-Cl-4-Me—Ph. |
| 17B | Q¹ is 2,6-di-Br-4-Me—Ph. |
| 18B | Q¹ is 2,4,6-tri-Cl—Ph. |
| 19B | Q¹ is 2-Cl-4-F—Ph. |
| 20B | Q¹ is 2-Cl-4-Me—Ph. |
| 21B | Q¹ is 2-Cl-4-MeO—Ph. |
| 22B | Q¹ is 2-Br-4-F—Ph. |
| 23B | Q¹ is 2-Br-4-Me—Ph. |
| 24B | Q¹ is 2-Br-4-MeO—Ph. |
| 25B | Q¹ is 2,4-di-Cl—Ph. |
| 26B | Q¹ is 2,6-di-Cl—Ph. |
| 27B | Q¹ is 2,4-di-Me—Ph. |
| 28B | Q¹ is 2,6-di-Me—Ph. |

TABLE 3

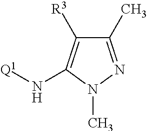

Q¹ is 2,4,6-tri-F—Ph
R³
CH₃
CH₃CH2
CH₃CH₂CH₂
(CH₃)₂CH
c-Pr
CH≡CCH₂—
CH₂═CH(CH₃)—
Bu
tert-Bu
(CH₃)₂CHCH₂
CH₃CH₂CH(CH₃)
c-Bu
CH₃CH═C(CH₃)—
CH≡CCH(CH₃)—
CH₂═CHCH(CH₃)—
pentyl
c-pentyl
CH₃C(CH₃)₂CH₂—
CH₃CH₂CH₂CH(CH₃)—
(CH₃CH₂)₂CH—
CH₃CH═C(Et)—
CH₂═CHCH(Et)—
CH₃CH₂CH═C(CH₃)—
CH₃CH═CHCH(CH₃)—
CH₃C≡CCH(CH₃)—
hexyl
c-hexyl
(CH₃)₂CHCH═C(Me)—
CH₃CH₂CH₂CH(Et)—
CH₃(CH₂)₃CH(CH₃)—
(CH₃)₂C═CHCH(CH₃)—
c-heptyl
c-octyl
CH₃OCH(CH₃)—
CH₃SCH(CH₃)—
CH₃OCH₂CH(CH₃)—
CH₃SCH₂CH(CH₃)—
CH₃CH₂OCH(CH₃)—
CH₃CH₂SCH(CH₃)—
CH₃O(CH₂)₂CH(CH₃)—
EtOCH₂CH(CH₃)—
CH₃CH₂CH₂OCH(Me)—
CF₃CH(CH₃)—
CF₃CH₂CH(CH₃)—
CH₃CH₂CH(CF₃)—
ClCH₂CH(CH₃)—

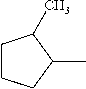

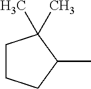

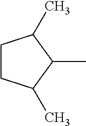

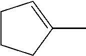

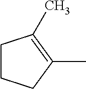

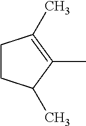

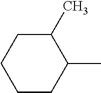

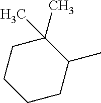

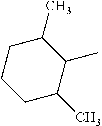

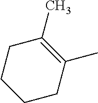

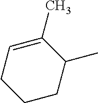

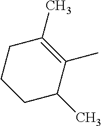

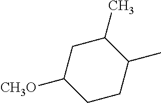

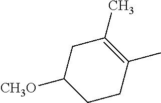

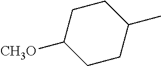

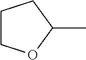

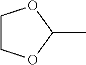

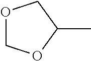

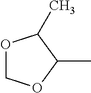

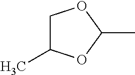

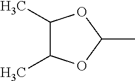

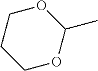

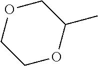

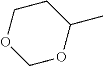

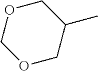

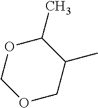

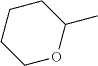

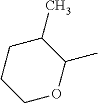

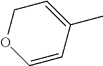

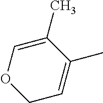

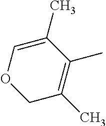

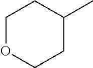

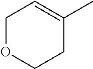

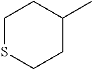

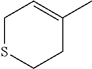

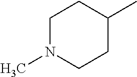

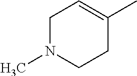

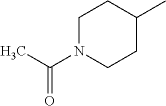

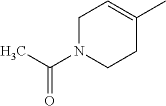

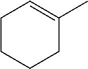

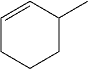

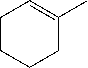

The present disclosure also includes Tables 1C through 28C, each of which is constructed the same as Table 3 above, except that the row heading in Table 3 (i.e. “Q¹ is 2,4,6-tri-F-Ph”) is replaced with the respective row headings shown below.

| Table | Row Heading |
|---|---|
| 1C | Q¹ is 2,6-di-F—Ph. |
| 2C | Q¹ is 2,6-di-F-4-MeO—Ph. |
| 3C | Q¹ is 2,6-di-F-4-Me—Ph. |
| 4C | Q¹ is 2,6-di-F-4-CN—Ph. |
| 5C | Q¹ is 2,6-di-F-4-Cl—Ph. |
| 6C | Q¹ is 2,6-di-F-4-Br—Ph. |
| 7C | Q¹ is 2,4-di-F—Ph. |
| 8C | Q¹ is 2,4-di-F-6-Cl—Ph. |
| 9C | Q¹ is 2,4-di-F-6-Br—Ph. |
| 10C | Q¹ is 2-Cl-6-F—Ph. |
| 11C | Q¹ is 2-Br-6-F—Ph. |
| 12C | Q¹ is 2-Cl-4-Me-6-F—Ph. |
| 13C | Q¹ is 2-Cl-4-MeO-6-F—Ph. |
| 14C | Q¹ is 2-Br-4-Me-6-F—Ph. |
| 15C | Q¹ is 2-Br-4-MeO-6-F—Ph. |
| 16C | Q¹ is 2,6-di-Cl-4-Me—Ph. |
| 17C | Q¹ is 2,6-di-Br-4-Me—Ph. |
| 18C | Q¹ is 2,4,6-tri-Cl—Ph. |
| 19C | Q¹ is 2-Cl-4-F—Ph. |
| 20C | Q¹ is 2-Cl-4-Me—Ph. |
| 21C | Q¹ is 2-Cl-4-MeO—Ph. |
| 22C | Q¹ is 2-Br-4-F—Ph. |
| 23C | Q¹ is 2-Br-4-Me—Ph. |
| 24C | Q¹ is 2-Br-4-MeO—Ph. |
| 25C | Q¹ is 2,4-di-Cl—Ph. |
| 26C | Q¹ is 2,6-di-Cl—Ph. |
| 27C | Q¹ is 2,4-di-Me—Ph. |
| 28C | Q¹ is 2,6-di-Me—Ph. |

TABLE 4

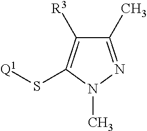

Q¹ is 2,4,6-tri-F—Ph
R³
CH₃
CH₃CH2
CH₃CH₂CH₂
(CH₃)₂CH
c-Pr
CH≡CCH₂—
CH₂═CH(CH₃)—
Bu
tert-Bu
(CH₃)₂CHCH₂
CH₃CH₂CH(CH₃)
c-Bu
CH₃CH═C(CH₃)—
CH≡CCH(CH₃)—
CH₂═CHCH(CH₃)—
pentyl
c-pentyl
CH₃C(CH₃)₂CH₂—
CH₃CH₂CH₂CH(CH₃)—
(CH₃CH₂)₂CH—
CH₃CH═C(Et)—
CH₂═CHCH(Et)—
CH₃CH₂CH═C(CH₃)—
CH₃CH═CHCH(CH₃)—
CH₃C≡CCH(CH₃)—
hexyl
c-hexyl
(CH₃)₂CHCH═C(Me)—
CH₃CH₂CH₂CH(Et)—
CH₃(CH₂)₃CH(CH₃)—
(CH₃)₂C═CHCH(CH₃)—
c-heptyl
c-octyl
CH₃OCH(CH₃)—
CH₃SCH(CH₃)—
CH₃OCH₂CH(CH₃)—
CH₃SCH₂CH(CH₃)—
CH₃CH₂OCH(CH₃)—
CH₃CH₂SCH(CH₃)—
CH₃O(CH₂)₂CH(CH₃)—
EtOCH₂CH(CH₃)—
CH₃CH₂CH₂OCH(Me)—
CF₃CH(CH₃)—
CF₃CH₂CH(CH₃)—
CH₃CH₂CH(CF₃)—
ClCH₂CH(CH₃)—

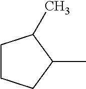

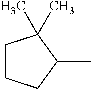

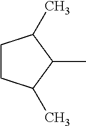

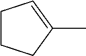

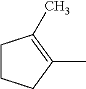

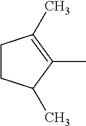

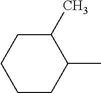

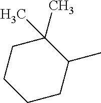

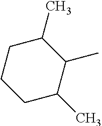

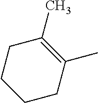

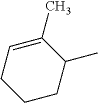

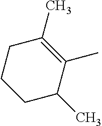

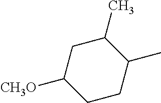

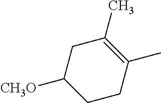

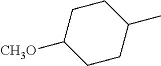

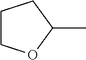

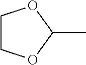

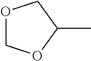

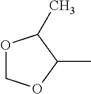

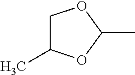

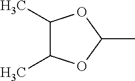

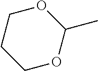

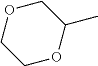

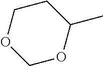

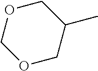

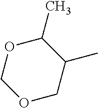

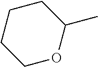

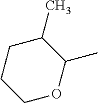

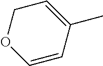

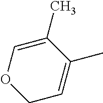

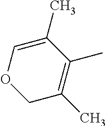

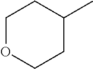

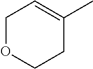

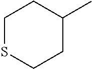

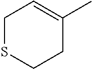

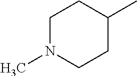

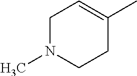

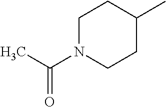

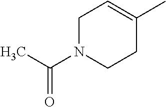

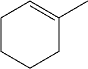

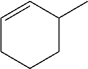

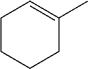

The present disclosure also includes Tables 1D through 28D, each of which is constructed the same as Table 4 above, except that the row heading in Table 4 (i.e. “Q¹ is 2,4,6-tri-F-Ph”) is replaced with the respective row headings shown below.

| Table | Row Heading |
|---|---|
| 1D | Q¹ is 2,6-di-F—Ph. |
| 2D | Q¹ is 2,6-di-F-4-MeO—Ph. |
| 3D | Q¹ is 2,6-di-F-4-Me—Ph. |
| 4D | Q¹ is 2,6-di-F-4-CN—Ph. |
| 5D | Q¹ is 2,6-di-F-4-Cl—Ph. |
| 6D | Q¹ is 2,6-di-F-4-Br—Ph. |
| 7D | Q¹ is 2,4-di-F—Ph. |
| 8D | Q¹ is 2,4-di-F-6-Cl—Ph. |
| 9D | Q¹ is 2,4-di-F-6-Br—Ph. |
| 10D | Q¹ is 2-Cl-6-F—Ph. |
| 11D | Q¹ is 2-Br-6-F—Ph. |
| 12D | Q¹ is 2-Cl-4-Me-6-F—Ph. |
| 13D | Q¹ is 2-Cl-4-MeO-6-F—Ph. |
| 14D | Q¹ is 2-Br-4-Me-6-F—Ph. |
| 15D | Q¹ is 2-Br-4-MeO-6-F—Ph. |
| 16D | Q¹ is 2,6-di-Cl-4-Me—Ph. |
| 17D | Q¹ is 2,6-di-Br-4-Me—Ph. |
| 18D | Q¹ is 2,4,6-tri-Cl—Ph. |
| 19D | Q¹ is 2-Cl-4-F—Ph. |
| 20D | Q¹ is 2-Cl-4-Me—Ph. |
| 21D | Q¹ is 2-Cl-4-MeO—Ph. |
| 22D | Q¹ is 2-Br-4-F—Ph. |
| 23D | Q¹ is 2-Br-4-Me—Ph. |
| 24D | Q¹ is 2-Br-4-MeO—Ph. |
| 25D | Q¹ is 2,4-di-Cl—Ph. |
| 26D | Q¹ is 2,6-di-Cl—Ph. |
| 27D | Q¹ is 2,4-di-Me—Ph. |
| 28D | Q¹ is 2,6-di-Me—Ph. |

### Formulation/Utility

A compound of Formula 1 of this invention (including N-oxides and salts thereof) will generally be used as a fungicidal active ingredient in a composition, i.e. formulation, with at least one additional component selected from the group consisting of surfactants, solid diluents and liquid diluents, which serve as a carrier. The formulation or composition ingredients are selected to be consistent with the physical properties of the active ingredient, mode of application and environmental factors such as soil type, moisture and temperature.

Useful formulations include both liquid and solid compositions. Liquid compositions include solutions (including emulsifiable concentrates), suspensions, emulsions (including microemulsions, oil-in-water emulsions, flowable concentrates and/or suspoemulsions) and the like, which optionally can be thickened into gels. The general types of aqueous liquid compositions are soluble concentrate, suspension concentrate, capsule suspension, concentrated emulsion, microemulsion, oil-in-water emulsion, flowable concentrate and suspo-emulsion. The general types of nonaqueous liquid compositions are emulsifiable concentrate, microemulsifiable concentrate, dispersible concentrate and oil dispersion.

The general types of solid compositions are dusts, powders, granules, pellets, prills, pastilles, tablets, filled films (including seed coatings) and the like, which can be water-dispersible (“wettable”) or water-soluble. Films and coatings formed from film-forming solutions or flowable suspensions are particularly useful for seed treatment. Active ingredient can be (micro)encapsulated and further formed into a suspension or solid formulation; alternatively the entire formulation of active ingredient can be encapsulated (or “overcoated”). Encapsulation can control or delay release of the active ingredient. An emulsifiable granule combines the advantages of both an emulsifiable concentrate formulation and a dry granular formulation. High-strength compositions are primarily used as intermediates for further formulation.

Sprayable formulations are typically extended in a suitable medium before spraying. Such liquid and solid formulations are formulated to be readily diluted in the spray medium, usually water, but occasionally another suitable medium like an aromatic or paraffinic hydrocarbon or vegetable oil. Spray volumes can range from about one to several thousand liters per hectare, but more typically are in the range from about ten to several hundred liters per hectare. Sprayable formulations can be tank mixed with water or another suitable medium for foliar treatment by aerial or ground application, or for application to the growing medium of the plant. Liquid and dry formulations can be metered directly into drip irrigation systems or metered into the furrow during planting. Liquid and solid formulations can be applied onto seeds of crops and other desirable vegetation as seed treatments before planting to protect developing roots and other subterranean plant parts and/or foliage through systemic uptake.

The formulations will typically contain effective amounts of active ingredient, diluent and surfactant within the following approximate ranges which add up to 100 percent by weight.

| | Weight Percent | | |
|---|---|---|---|
| | Active | | |
| | Ingredient | Diluent | Surfactant |
| Water-Dispersible and Water- | 0.001-90 | 0-99.999 | 0-15 |
| soluble Granules, Tablets and | | | |
| Powders | | | |
| Oil Dispersions, Suspensions, | 1-50 | 40-99 | 0-50 |
| Emulsions, Solutions | | | |
| (including Emulsifiable | | | |
| Concentrates) | | | |
| Dusts | 1-25 | 70-99 | 0-5 |
| Granules and Pellets | 0.001-95 | 5-99.999 | 0-15 |
| High Strength Compositions | 90-99 | 0-10 | 0-2 |

Solid diluents include, for example, clays such as bentonite, montmorillonite, attapulgite and kaolin, gypsum, cellulose, titanium dioxide, zinc oxide, starch, dextrin, sugars (e.g., lactose, sucrose), silica, talc, mica, diatomaceous earth, urea, calcium carbonate, sodium carbonate and bicarbonate, and sodium sulfate. Typical solid diluents are described in Watkins et al., Handbook of Insecticide Dust Diluents and Carriers, 2nd Ed., Dorland Books, Caldwell, New Jersey.

Liquid diluents include, for example, water, N,N-dimethylalkanamides (e.g., N,N-dimethylformamide), limonene, dimethyl sulfoxide, N-alkylpyrrolidones (e.g., N-methylpyrrolidinone), alkyl phosphates (e.g., triethyl phosphate), ethylene glycol, triethylene glycol, propylene glycol, dipropylene glycol, polypropylene glycol, propylene carbonate, butylene carbonate, paraffins (e.g., white mineral oils, normal paraffins, isoparaffins), alkylbenzenes, alkylnaphthalenes, glycerine, glycerol triacetate, sorbitol, aromatic hydrocarbons, dearomatized aliphatics, alkylbenzenes, alkylnaphthalenes, ketones such as cyclohexanone, 2-heptanone, isophorone and 4-hydroxy-4-methyl-2-pentanone, acetates such as isoamyl acetate, hexyl acetate, heptyl acetate, octyl acetate, nonyl acetate, tridecyl acetate and isobornyl acetate, other esters such as alkylated lactate esters, dibasic esters, alkyl and aryl benzoates and γ-butyrolactone, and alcohols, which can be linear, branched, saturated or unsaturated, such as methanol, ethanol, n-propanol, isopropyl alcohol, n-butanol, isobutyl alcohol, n-hexanol, 2-ethylhexanol, n-octanol, decanol, isodecyl alcohol, isooctadecanol, cetyl alcohol, lauryl alcohol, tridecyl alcohol, oleyl alcohol, cyclohexanol, tetrahydrofurfuryl alcohol, diacetone alcohol, cresol and benzyl alcohol. Liquid diluents also include glycerol esters of saturated and unsaturated fatty acids (typically C₆-C₂₂), such as plant seed and fruit oils (e.g., oils of olive, castor, linseed, sesame, corn (maize), peanut, sunflower, grapeseed, safflower, cottonseed, soybean, rapeseed, coconut and palm kernel), animal-sourced fats (e.g., beef tallow, pork tallow, lard, cod liver oil, fish oil), and mixtures thereof. Liquid diluents also include alkylated fatty acids (e.g., methylated, ethylated, butylated) wherein the fatty acids may be obtained by hydrolysis of glycerol esters from plant and animal sources, and can be purified by distillation. Typical liquid diluents are described in Marsden, Solvents Guide, 2nd Ed., Interscience, New York, 1950.

The solid and liquid compositions of the present invention often include one or more surfactants. When added to a liquid, surfactants (also known as “surface-active agents”) generally modify, most often reduce, the surface tension of the liquid. Depending on the nature of the hydrophilic and lipophilic groups in a surfactant molecule, surfactants can be useful as wetting agents, dispersants, emulsifiers or defoaming agents.

Surfactants can be classified as nonionic, anionic or cationic. Nonionic surfactants useful for the present compositions include, but are not limited to: alcohol alkoxylates such as alcohol alkoxylates based on natural and synthetic alcohols (which may be branched or linear) and prepared from the alcohols and ethylene oxide, propylene oxide, butylene oxide or mixtures thereof; amine ethoxylates, alkanolamides and ethoxylated alkanolamides; alkoxylated triglycerides such as ethoxylated soybean, castor and rapeseed oils; alkylphenol alkoxylates such as octylphenol ethoxylates, nonylphenol ethoxylates, dinonyl phenol ethoxylates and dodecyl phenol ethoxylates (prepared from the phenols and ethylene oxide, propylene oxide, butylene oxide or mixtures thereof); block polymers prepared from ethylene oxide or propylene oxide and reverse block polymers where the terminal blocks are prepared from propylene oxide; ethoxylated fatty acids; ethoxylated fatty esters and oils; ethoxylated methyl esters; ethoxylated tristyrylphenol (including those prepared from ethylene oxide, propylene oxide, butylene oxide or mixtures thereof); fatty acid esters, glycerol esters, lanolin-based derivatives, polyethoxylate esters such as polyethoxylated sorbitan fatty acid esters, polyethoxylated sorbitol fatty acid esters and polyethoxylated glycerol fatty acid esters; other sorbitan derivatives such as sorbitan esters; polymeric surfactants such as random copolymers, block copolymers, alkyd peg (polyethylene glycol) resins, graft or comb polymers and star polymers; polyethylene glycols (pegs); polyethylene glycol fatty acid esters; silicone-based surfactants; and sugar-derivatives such as sucrose esters, alkyl polyglycosides and alkyl polysaccharides.

Useful anionic surfactants include, but are not limited to: alkylaryl sulfonic acids and their salts; carboxylated alcohol or alkylphenol ethoxylates; diphenyl sulfonate derivatives; lignin and lignin derivatives such as lignosulfonates; maleic or succinic acids or their anhydrides; olefin sulfonates; phosphate esters such as phosphate esters of alcohol alkoxylates, phosphate esters of alkylphenol alkoxylates and phosphate esters of styryl phenol ethoxylates; protein-based surfactants; sarcosine derivatives; styryl phenol ether sulfate; sulfates and sulfonates of oils and fatty acids; sulfates and sulfonates of ethoxylated alkylphenols; sulfates of alcohols; sulfates of ethoxylated alcohols; sulfonates of amines and amides such as N,N-alkyltaurates; sulfonates of benzene, cumene, toluene, xylene, and dodecyl and tridecylbenzenes; sulfonates of condensed naphthalenes; sulfonates of naphthalene and alkyl naphthalene; sulfonates of fractionated petroleum; sulfosuccinamates; and sulfosuccinates and their derivatives such as dialkyl sulfosuccinate salts.

Useful cationic surfactants include, but are not limited to: amides and ethoxylated amides; amines such as N-alkyl propanediamines, tripropylenetriamines and dipropylenetetramines, and ethoxylated amines, ethoxylated diamines and propoxylated amines (prepared from the amines and ethylene oxide, propylene oxide, butylene oxide or mixtures thereof); amine salts such as amine acetates and diamine salts; quaternary ammonium salts such as quaternary salts, ethoxylated quaternary salts and diquaternary salts; and amine oxides such as alkyldimethylamine oxides and bis-(2-hydroxyethyl)-alkylamine oxides.

Also useful for the present compositions are mixtures of nonionic and anionic surfactants or mixtures of nonionic and cationic surfactants. Nonionic, anionic and cationic surfactants and their recommended uses are disclosed in a variety of published references including McCutcheon's Emulsifiers and Detergents, annual American and International Editions published by McCutcheon's Division, The Manufacturing Confectioner Publishing Co.; Sisely and Wood, Encyclopedia of Surface Active Agents, Chemical Publ. Co., Inc., New York, 1964; and A. S. Davidson and B. Milwidsky, Synthetic Detergents, Seventh Edition, John Wiley and Sons, New York, 1987.

Compositions of this invention may also contain formulation auxiliaries and additives, known to those skilled in the art as formulation aids (some of which may be considered to also function as solid diluents, liquid diluents or surfactants). Such formulation auxiliaries and additives may control: pH (buffers), foaming during processing (antifoams such polyorganosiloxanes), sedimentation of active ingredients (suspending agents), viscosity (thixotropic thickeners), in-container microbial growth (antimicrobials), product freezing (antifreezes), color (dyes/pigment dispersions), wash-off (film formers or stickers), evaporation (evaporation retardants), and other formulation attributes. Film formers include, for example, polyvinyl acetates, polyvinyl acetate copolymers, polyvinylpyrrolidone-vinyl acetate copolymer, polyvinyl alcohols, polyvinyl alcohol copolymers and waxes. Examples of formulation auxiliaries and additives include those listed in McCutcheon's Volume 2: Functional Materials, annual International and North American editions published by McCutcheon's Division, The Manufacturing Confectioner Publishing Co.; and PCT Publication WO 03/024222.

The compound of Formula 1 and any other active ingredients are typically incorporated into the present compositions by dissolving the active ingredient in a solvent or by grinding in a liquid or dry diluent. Solutions, including emulsifiable concentrates, can be prepared by simply mixing the ingredients. If the solvent of a liquid composition intended for use as an emulsifiable concentrate is water-immiscible, an emulsifier is typically added to emulsify the active-containing solvent upon dilution with water. Active ingredient slurries, with particle diameters of up to 2,000 μm can be wet milled using media mills to obtain particles with average diameters below 3 μm. Aqueous slurries can be made into finished suspension concentrates (see, for example, U.S. Pat. No. 3,060,084) or further processed by spray drying to form water-dispersible granules. Dry formulations usually require dry milling processes, which produce average particle diameters in the 2 to 10 μm range. Dusts and powders can be prepared by blending and usually grinding (such as with a hammer mill or fluid-energy mill). Granules and pellets can be prepared by spraying the active material upon preformed granular carriers or by agglomeration techniques. See Browning, “Agglomeration”, Chemical Engineering, Dec. 4, 1967, pp 147-48, Perry's Chemical Engineer's Handbook, 4th Ed., McGraw-Hill, New York, 1963, pages 8-57 and following, and WO 91/13546. Pellets can be prepared as described in U.S. Pat. No. 4,172,714. Water-dispersible and water-soluble granules can be prepared as taught in U.S. Pat. No. 4,144,050, U.S. Pat. No. 3,920,442 and DE 3,246,493. Tablets can be prepared as taught in U.S. Pat. No. 5,180,587, U.S. Pat. No. 5,232,701 and U.S. Pat. No. 5,208,030. Films can be prepared as taught in GB 2,095,558 and U.S. Pat. No. 3,299,566.

One embodiment of the present invention relates to a method for controlling fungal pathogens, comprising diluting the fungicidal composition of the present invention (a compound of Formula 1 formulated with surfactants, solid diluents and liquid diluents or a formulated mixture of a compound of Formula 1 and at least one other fungicide) with water, and optionally adding an adjuvant to form a diluted composition, and contacting the fungal pathogen or its environment with an effective amount of said diluted composition.

Although a spray composition formed by diluting with water a sufficient concentration of the present fungicidal composition can provide sufficient efficacy for controlling fungal pathogens, separately formulated adjuvant products can also be added to spray tank mixtures. These additional adjuvants are commonly known as “spray adjuvants” or “tank-mix adjuvants”, and include any substance mixed in a spray tank to improve the performance of a pesticide or alter the physical properties of the spray mixture. Adjuvants can be anionic or nonionic surfactants, emulsifying agents, petroleum-based crop oils, crop-derived seed oils, acidifiers, buffers, thickeners or defoaming agents. Adjuvants are used to enhancing efficacy (e.g., biological availability, adhesion, penetration, uniformity of coverage and durability of protection), or minimizing or eliminating spray application problems associated with incompatibility, foaming, drift, evaporation, volatilization and degradation. To obtain optimal performance, adjuvants are selected with regard to the properties of the active ingredient, formulation and target (e.g., crops, insect pests).

The amount of adjuvants added to spray mixtures is generally in the range of about 2.5% to 0.1% by volume. The application rates of adjuvants added to spray mixtures are typically between about 1 to 5 L per hectare. Representative examples of spray adjuvants include: Adigor® (Syngenta) 47% methylated rapeseed oil in liquid hydrocarbons, Silwet® (Helena Chemical Company) polyalkyleneoxide modified heptamethyltrisiloxane and Assist® (BASF) 17% surfactant blend in 83% paraffin based mineral oil.

One method of seed treatment is by spraying or dusting the seed with a compound of the invention (i.e. as a formulated composition) before sowing the seeds. Compositions formulated for seed treatment generally comprise a film former or adhesive agent. Therefore typically a seed coating composition of the present invention comprises a biologically effective amount of a compound of Formula 1 and a film former or adhesive agent. Seeds can be coated by spraying a flowable suspension concentrate directly into a tumbling bed of seeds and then drying the seeds. Alternatively, other formulation types such as wetted powders, solutions, suspoemulsions, emulsifiable concentrates and emulsions in water can be sprayed on the seed. This process is particularly useful for applying film coatings on seeds. Various coating machines and processes are available to one skilled in the art. Suitable processes include those listed in P. Kosters et al., Seed Treatment: Progress and Prospects, 1994 BCPC Mongraph No. 57, and references listed therein.

For further information regarding the art of formulation, see T. S. Woods, “The Formulator's Toolbox—Product Forms for Modern Agriculture” in Pesticide Chemistry and Bioscience, The Food-Environment Challenge, T. Brooks and T. R. Roberts, Eds., Proceedings of the 9th International Congress on Pesticide Chemistry, The Royal Society of Chemistry, Cambridge, 1999, pp. 120-133. See also U.S. Pat. No. 3,235,361, Col. 6, line 16 through Col. 7, line 19 and Examples 10-41; U.S. Pat. No. 3,309,192, Col. 5, line 43 through Col. 7, line 62 and Examples 8, 12, 15, 39, 41, 52, 53, 58, 132, 138-140, 162-164, 166, 167 and 169-182; U.S. Pat. No. 2,891,855, Col. 3, line 66 through Col. 5, line 17 and Examples 1-4; Klingman, Weed Control as a Science, John Wiley and Sons, Inc., New York, 1961, pp 81-96; Hance et al., Weed Control Handbook, 8th Ed., Blackwell Scientific Publications, Oxford, 1989; and Developments in formulation technology, PJB Publications, Richmond, U K, 2000.

In the following Examples, all percentages are by weight and all formulations are prepared in conventional ways. Compound numbers refer to compounds in Index Table A. Without further elaboration, it is believed that one skilled in the art using the preceding description can utilize the present invention to its fullest extent. The following Examples are, therefore, to be constructed as merely illustrative, and not limiting of the disclosure in any way whatsoever.

### Example A

High Strength Concentrate

| | |
|---|---|
| Compound 11 | 98.5% |
| silica aerogel | 0.5% |
| synthetic amorphous fine silica | 1.0% |

### Example B

Wettable Powder

| | |
|---|---|
| Compound 12 | 65.0% |
| dodecylphenol polyethylene glycol ether | 2.0% |
| sodium ligninsulfonate | 4.0% |
| sodium silicoaluminate | 6.0% |
| montmorillonite (calcined) | 23.0% |

### Example C

Granule

| | |
|---|---|
| Compound 24 | 10.0% |
| attapulgite granules (low volatile matter, 0.71/0.30 mm; | 90.0% |
| U.S.S. No. 25-50 sieves) | |

### Example D

Extruded Pellet

| | |
|---|---|
| Compound 46 | 25.0% |
| anhydrous sodium sulfate | 10.0% |
| crude calcium ligninsulfonate | 5.0% |
| sodium alkylnaphthalenesulfonate | 1.0% |
| calcium/magnesium bentonite | 59.0% |

### Example E

Emulsifiable Concentrate

| | |
|---|---|
| Compound 11 | 10.0% |
| polyoxyethylene sorbitol hexoleate | 20.0% |
| C₆-C₁₀ fatty acid methyl ester | 70.0% |

### Example F

Microemulsion

| | |
|---|---|
| Compound 52 | 5.0% |
| polyvinylpyrrolidone-vinyl acetate copolymer | 30.0% |
| alkylpolyglycoside | 30.0% |
| glyceryl monooleate | 15.0% |
| water | 20.0% |

### Example G

Seed Treatment

| | |
|---|---|
| Compound 11 | 20.00% |
| polyvinylpyrrolidone-vinyl acetate copolymer | 5.00% |
| montan acid wax | 5.00% |
| calcium ligninsulfonate | 1.00% |
| polyoxyethylene/polyoxypropylene block copolymers | 1.00% |
| stearyl alcohol (POE 20) | 2.00% |
| polyorganosilane | 0.20% |
| colorant red dye | 0.05% |
| water | 65.75% |

### Example H

Fertilizer Stick

| | |
|---|---|
| compound 12 | 2.50% |
| pyrrolidone-styrene copolymer | 4.80% |
| tristyrylphenyl 16-ethoxylate | 2.30% |
| talc | 0.80% |
| corn starch | 5.00% |
| slow-release fertilizer | 36.00% |
| kaolin | 38.00% |
| water | 10.60% |

### Example I

Suspension Concentrate

| | |
|---|---|
| compound 10 | 35% |
| butyl polyoxyethylene/polypropylene block copolymer | 4.0% |
| stearic acid/polyethylene glycol copolymer | 1.0% |
| styrene acrylic polymer | 1.0% |
| xanthan gum | 0.1% |
| propylene glycol | 5.0% |
| silicone based defoamer | 0.1% |
| 1,2-benzisothiazolin-3-one | 0.1% |
| water | 53.7% |

### Example J

Emulsion in Water

| | |
|---|---|
| compound 11 | 10.0% |
| butyl polyoxyethylene/polypropylene block copolymer | 4.0% |
| stearic acid/polyethylene glycol copolymer | 1.0% |
| styrene acrylic polymer | 1.0% |
| xanthan gum | 0.1% |
| propylene glycol | 5.0% |
| silicone based defoamer | 0.1% |
| 1,2-benzisothiazolin-3-one | 0.1% |
| aromatic petroleum based hydrocarbon | 20.0 |
| water | 58.7% |

### Example K

Oil Dispersion

| | |
|---|---|
| compound 24 | 25% |
| polyoxyethylene sorbitol hexaoleate | 15% |
| organically modified bentonite clay | 2.5% |
| fatty acid methyl ester | 57.5% |

### Example L

Suspoemulsion

| | |
|---|---|
| compound 46 | 10.0% |
| imidacloprid | 5.0% |
| butyl polyoxyethylene/polypropylene block copolymer | 4.0% |
| stearic acid/polyethylene glycol copolymer | 1.0% |
| styrene acrylic polymer | 1.0% |
| xanthan gum | 0.1% |
| propylene glycol | 5.0% |
| silicone based defoamer | 0.1% |
| 1,2-benzisothiazolin-3-one | 0.1% |
| aromatic petroleum based hydrocarbon | 20.0% |
| water | 53.7% |

Water-soluble and water-dispersible formulations are typically diluted with water to form aqueous compositions before application. Aqueous compositions for direct applications to the plant or portion thereof (e.g., spray tank compositions) typically contain at least about 1 ppm or more (e.g., from 1 ppm to 100 ppm) of the compound(s) of this invention.

Seed is normally treated at a rate of from about 0.001 g (more typically about 0.1 g) to about 10 g per kilogram of seed (i.e. from about 0.0001 to 1% by weight of the seed before treatment). A flowable suspension formulated for seed treatment typically comprises from about 0.5 to about 70% of the active ingredient, from about 0.5 to about 30% of a film-forming adhesive, from about 0.5 to about 20% of a dispersing agent, from 0 to about 5% of a thickener, from 0 to about 5% of a pigment and/or dye, from 0 to about 2% of an antifoaming agent, from 0 to about 1% of a preservative, and from 0 to about 75% of a volatile liquid diluent.

The compounds of this invention are useful as plant disease control agents. The present invention therefore further comprises a method for controlling plant diseases caused by fungal plant pathogens comprising applying to the plant or portion thereof to be protected, or to the plant seed to be protected, an effective amount of a compound of the invention or a fungicidal composition containing said compound. The compounds and/or compositions of this invention provide control of diseases caused by a broad spectrum of fungal plant pathogens in the Ascomycota, Basidiomycota, Zygomycota phyla, and the fungal-like Oomycata class. They are effective in controlling a broad spectrum of plant diseases, particularly foliar pathogens of ornamental, turf, vegetable, field, cereal, and fruit crops. These pathogens include but are not limited to those listed in Table 1-1. For Ascomycetes and Basidiomycetes, names for both the sexual/teleomorph/perfect stage as well as names for the asexual/anamorph/imperfect stage (in parentheses) are listed where known. Synonymous names for pathogens are indicated by an equal sign. For example, the sexual/teleomorph/perfect stage name Phaeosphaeria nodorum is followed by the corresponding asexual/anamorph/imperfect stage name Stagnospora nodorum and the synonymous older name Septoria nodorum.

TABLE 1-1
Ascomycetes in the order Pleosporales including Alternaria solani, A. alternata and
A. brassicae, Guignardia bidwellii, Venturia inaequalis, Pyrenophora tritici-repentis
(Dreschlera tritici-repentis = Helminthosporium tritici-repentis) and Pyrenophora teres
(Dreschlera teres = Helminthosporium teres), Corynespora cassiicola, Phaeosphaeria
nodorum (Stagonospora nodorum = Septoria nodorum), Cochliobolus carbonum and
C. heterostrophus, Leptosphaeria biglobosa and L. maculans;
Ascomycetes in the order Mycosphaerellales including Mycosphaerella graminicola
(Zymoseptoria tritici = Septoria tritici), M. berkeleyi (Cercosporidium personatum),
M. arachidis (Cercospora arachidicola), Passalora sojina (Cercospora sojina), Cercospora
zeae-maydis and C. beticola;
Ascomycetes in the order Erysiphales (the powdery mildews) such as Blumeria graminis
f. sp. tritici and Blumeria graminis f. sp. hordei, Erysiphe polygoni, E. necator (=Uncinula
necator), Podosphaera fuliginea (=Sphaerotheca fuliginea), and Podosphaera leucotricha
(=Sphaerotheca fuliginea);
Ascomycetes in the order Helotiales such as Botryotinia fuckeliana (Botrytis cinerea),
Oculimacula yallundae (=Tapesia yallundae; anamorph Helgardia herpotrichoides =
Pseudocercosporella herpetrichoides), Monilinia fructicola, Sclerotinia sclerotiorum,
Sclerotinia minor, and Sclerotinia homoeocarpa;
Ascomycetes in the order Hypocreales such as Giberella zeae (Fusarium graminearum),
G. monoliformis (Fusarium moniliforme), Fusarium solani and Verticillium dahliae;
Ascomycetes in the order Eurotiales such as Aspergillus flavus and A. parasiticus;
Ascomycetes in the order Diaporthales such as Cryptosphorella viticola (=Phomopsis
viticola), Phomopsis longicolla, and Diaporthe phaseolorum;
Other Ascomycete pathogens including Magnaporthe grisea, Gaeumannomyces graminis,
Rhynchosporium secalis, and anthracnose pathogens such as Glomerella acutata
(Colletotrichum acutatum), G. graminicola (C. graminicola) and G. lagenaria
(C. orbiculare);
Basidiomycetes in the order Urediniales (the rusts) including Puccinia recondita,
P. striiformis, Puccinia hordei, P. graminis and P. arachidis), Hemileia vastatrix and
Phakopsora pachyrhizi;
Basidiomycetes in the order Ceratobasidiales such as Thanatophorum cucumeris
(Rhizoctonia solani) and Ceratobasidium oryzae-sativae (Rhizoctonia oryzae);
Basidiomycetes in the order Polyporales such as Athelia rolfsii (Sclerotium rolfsii);
Basidiomycetes in the order Ustilaginales such as Ustilago maydis;
Zygomycetes in the order Mucorales such as Rhizopus stolonifer;
Oomycetes in the order Pythiales, including Phytophthora infestans, P. megasperma,
P. parasitica, P. sojae, P. cinnamomi and P. capsici, and Pythium pathogens such as Pythium
aphanidermatum, P. graminicola, P. irregulare, P. ultimum and P. dissoticum;
Oomycetes in the order Peronosporales such as Plasmopara viticola, P. halstedii,
Peronospora hyoscyami (=Peronospora tabacina), P. manshurica, Hyaloperonospora
parasitica (=Peronospora parasitica), Pseudoperonospora cubensis and Bremia lactucae;
and other genera and species closely related to all of the above pathogens.

In addition to their fungicidal activity, the compositions or combinations also have activity against bacteria such as Erwinia amylovora, Xanthomonas campestris, Pseudomonas syringae, and other related species. By controlling harmful microorganisms, the compounds of the invention are useful for improving (i.e. increasing) the ratio of beneficial to harmful microorganisms in contact with crop plants or their propagules (e.g., seeds, corms, bulbs, tubers, cuttings) or in the agronomic environment of the crop plants or their propagules.

Compounds of the invention are useful in treating all plants, plant parts and seeds. Plant and seed varieties and cultivars can be obtained by conventional propagation and breeding methods or by genetic engineering methods. Genetically modified plants or seeds (transgenic plants or seeds) are those in which a heterologous gene (transgene) has been stably integrated into the plant's or seed's genome. A transgene that is defined by its particular location in the plant genome is called a transformation or transgenic event.

Genetically modified plant cultivars which can be treated according to the invention include those that are resistant against one or more biotic stresses (pests such as nematodes, insects, mites, fungi, etc.) or abiotic stresses (drought, cold temperature, soil salinity, etc.), or that contain other desirable characteristics. Plants can be genetically modified to exhibit traits of, for example, herbicide tolerance, insect-resistance, modified oil profiles or drought tolerance. Useful genetically modified plants containing single gene transformation events or combinations of transformation events are listed in Table 2-1. Additional information for the genetic modifications listed in Table 2-1 can be obtained from publicly available databases maintained, for example, by the U.S. Department of Agriculture.

The following abbreviations are used in Table 2-1 for traits. A dash (“-”) means the entry is not available.

| Trait | Description |
|---|---|
| T1 | Glyphosate tolerance |
| T2 | High lauric acid oil |
| T3 | Glufosinate tolerance |
| T4 | Phytate breakdown |
| T5 | Oxynil tolerance |
| T6 | Disease resistance |
| T7 | Insect resistance |
| T9 | Modified flower color |
| T11 | ALS herbicide tol. |
| T12 | Dicamba tolerance |
| T13 | Anti-allergy |
| T14 | Salt tolerance |
| T15 | Cold tolerance |
| T16 | Imidazolinone herbicide tol. |
| T17 | Modified alpha-amylase |
| T18 | Pollination control |
| T19 | 2,4-D tolerance |
| T20 | Increased lysine |
| T21 | Drought tolerance |
| T22 | Delayed ripening/senescence |
| T23 | Modified product quality |
| T24 | High cellulose |
| T25 | Modified starch/carbohydrate |
| T26 | Insect & disease resist. |
| T27 | High tryptophan |
| T28 | Erect leaves semidwarf |
| T29 | Semidwarf |
| T30 | Low iron tolerance |
| T31 | Modified oil/fatty acid |
| T32 | HPPD tolerance |
| T33 | High oil |
| T34 | Aryloxyalkanoate tol. |
| T35 | Mesotrione tolerance |
| T36 | Reduced nicotine |
| T37 | Modified product |

TABLE 2-1

| Crop | Event Name | Event Code | Trait(s) | Gene(s) |
|---|---|---|---|---|
| Alfalfa | J101 | MON-00101-8 | T1 | cp4 epsps (aroA:CP4) |
| Alfalfa | J163 | MON-ØØ163-7 | T1 | cp4 epsps (aroA:CP4) |
| Canola* | 23-18-17 (Event 18) | CGN-89465-2 | T2 | te |
| Canola* | 23-198 (Event 23) | CGN-89465-2 | T2 | te |
| Canola* | 61061 | DP-Ø61Ø61-7 | T1 | gat4621 |
| Canola* | 73496 | DP-Ø73496-4 | T1 | gat4621 |
| Canola* | GT200 (RT200) | MON-89249-2 | T1 | cp4 epsps (aroA:CP4); goxv247 |
| Canola* | GT73 (RT73) | MON-ØØØ73-7 | T1 | cp4 epsps (aroA:CP4); goxv247 |
| Canola* | HCN10 (Topas 19/2) | — | T3 | bar |
| Canola* | HCN28 (T45) | ACS-BNØØ8-2 | T3 | pat (syn) |
| Canola* | HCN92 (Topas 19/2) | ACS-BNØØ7-1 | T3 | bar |
| Canola* | MON88302 | MON-883Ø2-9 | T1 | cp4 epsps (aroA:CP4) |
| Canola* | MPS961 | — | T4 | phyA |
| Canola* | MPS962 | — | T4 | phyA |
| Canola* | MPS963 | — | T4 | phyA |
| Canola* | MPS964 | — | T4 | phyA |
| Canola* | MPS965 | — | T4 | phyA |
| Canola* | MS1 (B91-4) | ACS-BNØØ4-7 | T3 | bar |
| Canola* | MS8 | ACS-BNØØ5-8 | T3 | bar |
| Canola* | OXY-235 | ACS-BNØ11-5 | T5 | bxn |
| Canola* | PHY14 | — | T3 | bar |
| Canola* | PHY23 | — | T3 | bar |
| Canola* | PHY35 | — | T3 | bar |
| Canola* | PHY36 | — | T3 | bar |
| Canola* | RF1 (B93-101) | ACS-BNØØ1-4 | T3 | bar |
| Canola* | RF2 (B94-2) | ACS-BNØØ2-5 | T3 | bar |
| Canola* | RF3 | ACS-BNØØ3-6 | T3 | bar |
| Bean | EMBRAPA 5.1 | EMB-PV051-1 | T6 | ac1 (sense and antisense) |
| Brinjal# | EE-1 | — | T7 | cry1Ac |
| Carnation | 11 (7442) | FLO-07442-4 | T8; T9 | surB; dfr; hfl (f3′5′h) |
| Carnation | 11363 (1363A) | FLO-11363-1 | T8; T9 | surB; dfr; bp40 (f3′5′h) |
| Carnation | 1226A (11226) | FLO-11226-8 | T8; T9 | surB; dfr; bp40 (f3′5′h) |
| Carnation | 123.2.2 (40619) | FLO-4Ø619-7 | T8; T9 | surB; dfr; hfl (f3′5′h) |
| Carnation | 123.2.38 (40644) | FLO-4Ø644-4 | T8; T9 | surB; dfr; hfl (f3′5′h) |
| Carnation | 123.8.12 | FLO-4Ø689-6 | T8; T9 | surB; dfr; bp40 (f3′5′h) |
| Carnation | 123.8.8 (40685) | FLO-4Ø685-1 | T8; T9 | surB; dfr; bp40 (f3′5′h) |
| Carnation | 1351A (11351) | FLO-11351-7 | T8; T9 | surB; dfr; bp40 (f3′5′h) |
| Carnation | 1400A (11400) | FLO-114ØØ-2 | T8; T9 | surB; dfr; bp40 (f3′5′h) |
| Carnation | 15 | FLO-ØØØ15-2 | T8; T9 | surB; dfr; hfl (f3′5′h) |
| Carnation | 16 | FLO-ØØØ16-3 | T8; T9 | surB; dfr; hfl (f3′5′h) |
| Carnation | 4 | FLO-ØØØØ4-9 | T8; T9 | surB; dfr; hfl (f3′5′h) |
| Carnation | 66 | FLO-ØØØ66-8 | T8; T10 | surB; acc |
| Carnation | 959A (11959) | FLO-11959-3 | T8; T9 | surB; dfr; bp40 (f3′5′h) |
| Carnation | 988A (11988) | FLO-11988-7 | T8; T9 | surB; dfr; bp40 (f3′5′h) |
| Carnation | 26407 | IFD-26497-2 | ST8; T9 | surB; dfr; bp40 (f3′5′h) |
| Carnation | 25958 | IFD-25958-3 | T8; T9 | surB; dfr; bp40 (f3′5′h) |
| Chicory | RM3-3 | — | T3 | bar |
| Chicory | RM3-4 | — | T3 | bar |
| Chicory | RM3-6 | — | T3 | bar |
| Cotton | 19-51a | DD-Ø1951A-7 | T11 | S4-HrA |
| Cotton | 281-24-236 | DAS-24236-5 | T3; T7 | pat (syn); cry1F |
| Cotton | 3006-210-23 | DAS-21Ø23-5 | T3; T7 | pat (syn); cry1Ac |
| Cotton | 31707 | — | T5; T7 | bxn; cry1Ac |
| Cotton | 31803 | — | T5; T7 | bxn; cry1Ac |
| Cotton | 31807 | — | T5; T7 | bxn; cry1Ac |
| Cotton | 31808 | — | T5; T7 | bxn; cry1Ac |
| Cotton | 42317 | — | T5; T7 | bxn; cry1Ac |
| Cotton | BNLA-601 | — | T7 | cry1Ac |
| Cotton | BXN10211 | BXN10211-9 | T5 | bxn; cry1Ac |
| Cotton | BXN10215 | BXN10215-4 | T5 | bxn; cry1Ac |
| Cotton | BXN10222 | BXN10222-2 | T5 | bxn; cry1Ac |
| Cotton | BXN10224 | BXN10224-4 | T5 | bxn; cry1Ac |
| Cotton | COT102 | SYN-IR102-7 | T7 | vip3A(a) |
| Cotton | COT67B | SYN-IR67B-1 | T7 | cry1Ab |
| Cotton | COT202 | — | T7 | vip3A |
| Cotton | Event 1 | — | T7 | cry1Ac |
| Cotton | GMF Cry1A | GTL-GMF311-7 | T7 | cry1Ab-Ac |
| Cotton | GHB119 | BCS-GH005-8 | T7 | cry2Ae |
| Cotton | GHB614 | BCS-GH002-5 | T1 | 2mepsps |
| Cotton | GK12 | — | T7 | cry1Ab-Ac |
| Cotton | LLCotton25 | ACS-GH001-3 | T3 | bar |
| Cotton | MLS 9124 | — | T7 | cry1C |
| Cotton | MON1076 | MON-89924-2 | T7 | cry1Ac |
| Cotton | MON1445 | MON-01445-2 | T1 | cp4 epsps (aroA:CP4) |
| Cotton | MON15985 | MON-15985-7 | T7 | cry1Ac; cry2Ab2 |
| Cotton | MON1698 | MON-89383-1 | T7 | cp4 epsps (aroA:CP4) |
| Cotton | MON531 | MON-00531-6 | T7 | cry1Ac |
| Cotton | MON757 | MON-00757-7 | T7 | cry1Ac |
| Cotton | MON88913 | MON-88913-8 | T1 | cp4 epsps (aroA:CP4) |
| Cotton | Nqwe Chi 6 Bt | — | T7 | — |
| Cotton | SKG321 | — | T7 | cry1A; CpTI |
| Cotton | T303-3 | BCS-GH003-6 | T7; T3 | cry1Ab; bar |
| Cotton | T304-40 | BCS-GH004-7 | T7; T3 | cry1Ab; bar |
| Cotton | CE43-67B | — | T7 | cry1Ab |
| Cotton | CE46-02A | — | T7 | cry1Ab |
| Cotton | CE44-69D | — | T7 | cry1Ab |
| Cotton | 1143-14A | — | T7 | cry1Ab |
| Cotton | 1143-51B | — | T7 | cry1Ab |
| Cotton | T342-142 | — | T7 | cry1Ab |
| Cotton | PV-GHGT07 (1445) | — | T1 | cp4 epsps (aroA:CP4) |
| Cotton | EE-GH3 | — | T1 | mepsps |
| Cotton | EE-GH5 | — | T7 | cry1Ab |
| Cotton | MON88701 | MON-88701-3 | T12; T3 | Modified dmo; bar |
| Cotton | OsCr11 | — | T13 | Modified Cry j |
| Creeping | ASR368 | SMG-368ØØ-2 | T1 | cp4 epsps (aroA:CP4) |
| Bentgrass | | | | |
| Eucalyptus | 20-C | — | T14 | codA |
| Eucalyptus | 12-5C | — | T14 | codA |
| Eucalyptus | 12-5B | — | T14 | codA |
| Eucalyptus | 107-1 | — | T14 | codA |
| Eucalyptus | 1/9/2001 | — | T14 | codA |
| Eucalyptus | 2/1/2001 | — | T14 | codA |
| Eucalyptus | | — | T15 | des9 |
| Flax | FP967 | CDC-FL001-2 | T11 | als |
| Lentil | RH44 | — | T16 | als |
| Maize | 3272 | SYN-E3272-5 | T17 | amy797E |
| Maize | 5307 | SYN-05307-1 | T7 | ecry3.1Ab |
| Maize | 59122 | DAS-59122-7 | T7; T3 | cry34Ab1; cry35Ab1; pat |
| Maize | 676 | PH-000676-7 | T3; T18 | pat; dam |
| Maize | 678 | PH-000678-9 | T3; T18 | pat; dam |
| Maize | 680 | PH-000680-2 | T3; T18 | pat; dam |
| Maize | 98140 | DP-098140-6 | T1; T11 | gat4621; zm-hra |
| Maize | Bt10 | — | T7; T3 | cry1Ab; pat |
| Maize | Bt176 (176) | SYN-EV176-9 | T7; T3 | cry1Ab; bar |
| Maize | BVLA430101 | — | T4 | phyA2 |
| Maize | CBH-351 | ACS-ZM004-3 | T7; T3 | cry9C; bar |
| Maize | DAS40278-9 | DAS40278-9 | T19 | aad-1 |
| Maize | DBT418 | DKB-89614-9 | T7; T3 | cry1Ac; pinII; bar |
| Maize | DLL25 (B16) | DKB-89790-5 | T3 | bar |
| Maize | GA21 | MON-00021-9 | T1 | mepsps |
| Maize | GG25 | — | T1 | mepsps |
| Maize | GJ11 | — | T1 | mepsps |
| Maize | Fl117 | — | T1 | mepsps |
| Maize | GAT-ZM1 | — | T3 | pat |
| Maize | LY038 | REN-00038-3 | T20 | cordapA |
| Maize | MIR162 | SYN-IR162-4 | T7 | vip3Aa20 |
| Maize | MIR604 | SYN-IR604-5 | T7 | mcry3A |
| Maize | MON801 | MON801 | T7; T1 | cry1Ab; cp4 epsps (aroA:CP4); |
| | (MON80100) | | | goxv247 |
| Maize | MON802 | MON-80200-7 | T7; T1 | cry1Ab; cp4 epsps (aroA:CP4); |
| | | | | goxv247 |
| Maize | MON809 | PH-MON-809-2 | T7; T1 | cry1Ab; cp4 epsps (aroA:CP4); |
| | | | | goxv247 |
| Maize | MON810 | MON-00810-6 | T7; T1 | cry1Ab; cp4 epsps (aroA:CP4); |
| | | | | goxv247 |
| Maize | MON832 | — | T1 | cp4 epsps (aroA:CP4); goxv247 |
| Maize | MON863 | MON-00863-5 | T7 | cry3Bb1 |
| Maize | MON87427 | MON-87427-7 | T1 | cp4 epsps (aroA:CP4) |
| Maize | MON87460 | MON-87460-4 | T21 | cspB |
| Maize | MON88017 | MON-88017-3 | T7; T1 | cry3Bb1; cp4 epsps (aroA:CP4) |
| Maize | MON89034 | MON-89034-3 | T7 | cry2Ab2; cry1A.105 |
| Maize | MS3 | ACS-ZM001-9 | T3; T18 | bar; bar-se |
| Maize | MS6 | ACS-ZM005-4 | T3; T18 | bar; bar-se |
| Maize | NK603 | MON-00603-6 | T1 | cp4 epsps (aroA:CP4) |
| Maize | T14 | ACS-ZM002-1 | T3 | pat (syn) |
| Maize | T25 | ACS-ZM003-2 | T3 | pat (syn) |
| Maize | TC1507 | DAS-01507-1 | T7; T3 | cry1Fa2; pat |
| Maize | TC6275 | DAS-06275-8 | T7; T3 | mocry1F; bar |
| Maize | VIP1034 | | T7; T3 | vip3A; pat |
| Maize | 43A47 | DP-043A47-3 | T7; T3 | cry1F; cry34Ab1; cry35Ab1; pat |
| Maize | 40416 | DP-040416-8 | T7; T3 | cry1F; cry34Ab1; cry35Ab1; pat |
| Maize | 32316 | DP-032316-8 | T7; T3 | cry1F; cry34Ab1; cry35Ab1; pat |
| Maize | 4114 | DP-004114-3 | T7; T3 | cry1F; cry34Ab1; cry35Ab1; pat |
| Melon | Melon A | — | T22 | sam-k |
| Melon | Melon B | — | T22 | sam-k |
| Papaya | 55-1 | CUH-CP551-8 | T6 | prsv cp |
| Papaya | 63-1 | CUH-CP631-7 | T6 | prsv cp |
| Papaya | Huanong No. 1 | — | T6 | prsv rep |
| Papaya | X17-2 | UFL-X17CP-6 | T6 | prsv cp |
| Petunia | Petunia-CHS | — | T25 | CHS suppres.sion |
| Plum | C-5 | ARS-PLMC5-6 | T6 | ppv cp |
| Canola** | ZSR500 | — | T1 | cp4 epsps (aroA:CP4); goxv247 |
| Canola** | ZSR502 | — | T1 | cp4 epsps (aroA:CP4); goxv247 |
| Canola** | ZSR503 | — | T1 | cp4 epsps (aroA:CP4); goxv247 |
| Poplar | Bt poplar | — | T7 | cry1Ac; API |
| Poplar | Hybrid poplar | — | T7 | cry1Ac; API |
| | clone 741 | | | |
| Poplar | trg300-l | — | T24 | AaXEG2 |
| Poplar | trg300-2 | — | T24 | AaXEG2 |
| Potato | 1210 amk | — | T7 | cry3A |
| Potato | 2904/1 kgs | — | T7 | cry3A |
| Canola** | ZSR500 | — | T1 | cp4 epsps (aroA:CP4); goxv247 |
| Canola** | ZSR502 | — | T1 | cp4 epsps (aroA:CP4); goxv247 |
| Potato | ATBT04-27 | NMK-89367-8 | T7 | cry3A |
| Potato | ATBT04-30 | NMK-89613-2 | T7 | cry3A |
| Potato | ATBT04-31 | NMK-89170-9 | T7 | cry3A |
| Potato | ATBT04-36 | NMK-89279-1 | T7 | cry3A |
| Potato | ATBT04-6 | NMK-89761-6 | T7 | cry3A |
| Potato | BT06 | NMK-89812-3 | T7 | cry3A |
| Potato | BT10 | NMK-89175-5 | T7 | cry3A |
| Potato | BT12 | NMK-89601-8 | T7 | cry3A |
| Potato | BT16 | NMK-89167-6 | T7 | cry3A |
| Potato | BT17 | NMK-89593-9 | T7 | cry3A |
| Potato | BT18 | NMK-89906-7 | T7 | cry3A |
| Potato | BT23 | NMK-89675-1 | T7 | cry3A |
| Potato | EH92-527-1 | BPS-25271-9 | T25 | gbss (antisense) |
| Potato | HLMT15-15 | — | T7; T6 | cry3A; pvy cp |
| Potato | HLMT15-3 | — | T7; T6 | cry3A; pvy cp |
| Potato | HLMT15-46 | — | T7; T6 | cry3A; pvy cp |
| Potato | RBMT15-101 | NMK-89653-6 | T7; T6 | cry3A; pvy cp |
| Potato | RBMT21-129 | NMK-89684-1 | T7; T6 | cry3A; plrv orf1; plrv orf2 |
| Potato | RBMT21-152 | — | T7; T6 | cry3A; plrv orf1; plrv orf2 |
| Potato | RBMT21-350 | NMK-89185-6 | T7; T6 | cry3A; plrv orf1; plrv orf2 |
| Potato | RBMT22-082 | NMK-89896-6 | T7; T6.; T1 | cry3A; plrv orf1; plrv orf2; cp4 |
| | | | | epsps (aroA:CP4) |
| Potato | RBMT22-186 | — | T7; T6.; T1 | cry3A; plrv orf1; plrv orf2; cp4 |
| | | | | epsps (aroA:CP4) |
| Potato | RBMT22-238 | — | T7; T6.; T1 | cry3A; plrv orf1; plrv orf2; cp4 |
| | | | | epsps (aroA:CP4) |
| Potato | RBMT22-262 | — | T7; T6.; T1 | cry3A; plrv orf1; plrv orf2; cp4 |
| | | | | epsps (aroA:CP4) |
| Potato | SEMT15-02 | NMK-89935-9 | T7; T6 | cry3A; pvy cp |
| Potato | SEMT15-07 | — | T7; T6 | cry3A; pvy cp |
| Potato | SEMT15-15 | NMK-89930-4 | T7; T6 | cry3A; pvy cp |
| Potato | SPBT02-5 | NMK-89576-1 | T7 | cry3A |
| Potato | SPBT02-7 | NMK-89724-5 | T7 | cry3A |
| Rice | 7Crp#242-95-7 | — | T13 | 7crp |
| Rice | 7Crp#10 | — | T13 | 7crp |
| Rice | GM Shanyou 63 | — | T7 | cry1Ab; cry1Ac |
| Rice | Huahui-1/TT51-1 | — | T7 | cry1Ab; cry1Ac |
| Rice | LLRICE06 | ACS-OS001-4 | T3 | bar |
| Rice | LLRICE601 | BCS-OS003-7 | T3 | bar |
| Rice | LLRICE62 | ACS-OS002-5 | T3 | bar |
| Rice | Tarom molaii + | — | T7 | cry1Ab (truncated) |
| | cry1Ab | | | |
| Rice | GAT-OS2 | — | T3 | bar |
| Rice | GAT-OS3 | — | T3 | bar |
| Rice | PE-7 | — | T7 | Cry1Ac |
| Rice | 7Crp#10 | — | T13 | 7crp |
| Rice | KPD627-8 | — | T27 | OASA1D |
| Rice | KPD722-4 | — | T27 | OASA1D |
| Rice | KA317 | — | T27 | OASA1D |
| Rice | HW5 | — | T27 | OASA1D |
| Rice | HW1 | — | T27 | OASA1D |
| Rice | B-4-1-18 | — | T28 | Δ OsBRI1 |
| Rice | G-3-3-22 | — | T29 | OSGA2ox1 |
| Rice | AD77 | — | T6 | DEF |
| Rice | AD51 | — | T6 | DEF |
| Rice | AD48 | — | T6 | DEF |
| Rice | AD41 | — | T6 | DEF |
| Rice | 13p-s-atAprt1 | — | T30 | Hv-S1; Hv-AT-A; APRT |
| Rice | 13pAprt1 | — | T30 | APRT |
| Rice | gHv-S1-gHv-AT-1 | — | T30 | Hv-S1; Hv-AT-A; Hv-AT-B |
| Rice | gHvIDS3-1 | — | T30 | HvIDS3 |
| Rice | gHv-AT1 | — | T30 | Hv-AT-A; Hv-AT-B |
| Rice | gHv-S1-1 | — | T30 | Hv-S1 |
| Rice | NIA-OS006-4 | — | T6 | WRKY45 |
| Rice | NIA-OS005-3 | — | T6 | WRKY45 |
| Rice | NIA-OS004-2 | — | T6 | WRKY45 |
| Rice | NIA-OS003-1 | — | T6 | WRKY45 |
| Rice | NIA-OS002-9 | — | T6 | WRKY45 |
| Rice | NIA-OS001-8 | — | T6 | WRKY45 |
| Rice | OsCr11 | — | T13 | Modified Cry j |
| Rice | 17053 | — | T1 | cp4 epsps (aroA:CP4) |
| Rice | 17314 | — | T1 | cp4 epsps (aroA:CP4) |
| Rose | WKS82/130-4-1 | IFD-52401-4 | T9 | 5AT; bp40 (f3′5′h) |
| Rose | WKS92/130-9-1 | IFD-52901-9 | T9 | 5AT; bp40 (f3′5′h) |
| Soybean | 260-05 (G94-1, | — | T9 | gm-fad2-1 (silencing locus) |
| | G94-19, G168) | | | |
| Soybean | A2704-12 | ACS-GM005-3 | T3 | pat |
| Soybean | A2704-21 | ACS-GM004-2 | T3 | pat |
| Soybean | A5547-127 | ACS-GM006-4 | T3 | pat |
| Soybean | A5547-35 | ACS-GM008-6 | T3 | pat |
| Soybean | CV127 | BPS-CV127-9 | T16 | csr1-2 |
| Soybean | DAS68416-4 | DAS68416-4 | T3 | pat |
| Soybean | DP305423 | DP-305423-1 | T31; T11 | gm-fad2-1 (silencing locus); gm-hra |
| Soybean | DP356043 | DP-356043-5 | T31; T1 | gm-fad2-1 (silencing locus); gat4601 |
| Soybean | FG72 | MST-FG072-3 | T1; T32 | 2mepsps; hppdPF W336 |
| Soybean | GTS 40-3-2 (40-3-2) | MON-04032-6 | T1 | cp4 epsps (aroA:CP4) |
| Soybean | GU262 | ACS-GM003-1 | T3 | pat |
| Soybean | MON87701 | MON-87701-2 | T7 | cry1Ac |
| Soybean | MON87705 | MON-87705-6 | T31; T1 | fatb1-A (sense & antisense); fad2- |
| | | | | 1A (sense & antisense); cp4 epsps |
| | | | | (aroA:CP4) |
| Soybean | MON87708 | MON-87708-9 | T12; T1 | dmo; cp4 epsps (aroA:CP4) |
| Soybean | MON87769 | MON-87769-7 | T31; T1 | Pj.D6D; Nc.Fad3; cp4 epsps |
| | | | | (aroA:CP4) |
| Soybean | MON89788 | MON-89788-1 | T1 | cp4 epsps (aroA:CP4) |
| Soybean | W62 | ACS-GM002-9 | T3 | bar |
| Soybean | W98 | ACS-GM001-8 | T3 | bar |
| Soybean | MON87754 | MON-87754-1 | T33 | dgat2A |
| Soybean | DAS21606 | DAS-21606 | T34; T3 | Modified aad-12; pat |
| Soybean | DAS44406 | DAS-44406-6 | T34; T1; T3 | Modified aad-12; 2mepsps; pat |
| Soybean | SYHT04R | SYN-0004R-8 | T35 | Modified avhppd |
| Soybean | 9582.814.19.1 | | T7; T3 | cry1Ac; cry1F; pat |
| Squash | CZW3 | SEM-ØCZW3-2 | T6 | cmv cp; zymv cp; wmv cp |
| Squash | ZW20 | SEM-0ZW20-7 | T6 | zymv cp; wmv cp |
| Sugar Beet | GTSB77 | SY-GTSB77-8 | T1 | cp4 epsps (aroA:CP4); goxv247 |
| | (T9100152) | | | |
| Sugar Beet | H7-1 | KM-000H71-4 | T1 | cp4 epsps (aroA:CP4) |
| Sugar Beet | T120-7 | ACS-BV001-3 | T3 | pat |
| Sugar Beet | T227-1 | — | T1 | cp4 epsps (aroA:CP4) |
| Sugarcane | NXI-1T | — | T21 | EcbetA |
| Sunflower | X81359 | — | T16 | als |
| Sweet Pepper | PK-SP01 | — | T6 | cmv cp |
| Tobacco | C/F/93/08-02 | — | T5 | bxn |
| Tobacco | Vector 21-41 | — | T36 | NtQPT1 (antisense) |
| Tomato | 1345-4 | — | T22 | acc (truncated) |
| Tomato | 35-1-N | — | T22 | sam-k |
| Tomato | 5345 | — | T7 | cry1Ac |
| Tomato | 8338 | CGN-89322-3 | T22 | accd |
| Tomato | B | SYN-0000B-6 | T22 | pg (sense or antisense) |
| Tomato | Da | SYN-0000DA-9 | T22 | pg (sense or antisense) |
| Sunflower | X81359 | — | T16 | als |
| Tomato | Da Dong No 9 | — | T37 | — |
| Tomato | F (1401F, h38F, | SYN-0000F-1 | T22 | pg (sense or antisense) |
| | 11013F, 7913F) | | | |
| Tomato | FLAVR SAVR ™ | CGN-89564-2 | T22 | pg (sense or antisense) |
| Tomato | Huafan No 1 | — | T22 | anti-efe |
| Tomato | PK-TM8805R | — | T6 | cmv cp |
| | (8805R) | | | |
| Wheat | MON71800 | MON-718ØØ-3 | T1 | cp4 epsps (aroA:CP4) |

*Argentine,
**Polish,
#Eggplant

Treatment of genetically modified plants and seeds with compounds of the invention may result in super-additive or synergistic effects. For example, reduction in application rates, broadening of the activity spectrum, increased tolerance to biotic/abiotic stresses or enhanced storage stability may be greater than expected from just simple additive effects of the application of compounds of the invention on genetically modified plants and seeds.

Compounds of this invention are useful in seed treatments for protecting seeds from plant diseases. In the context of the present disclosure and claims, treating a seed means contacting the seed with a biologically effective amount of a compound of this invention, which is typically formulated as a composition of the invention. This seed treatment protects the seed from soil-borne disease pathogens and generally can also protect roots and other plant parts in contact with the soil of the seedling developing from the germinating seed. The seed treatment may also provide protection of foliage by translocation of the compound of this invention or a second active ingredient within the developing plant. Seed treatments can be applied to all types of seeds, including those from which plants genetically transformed to express specialized traits will germinate. Representative examples include those expressing proteins toxic to invertebrate pests, such as Bacillus thuringiensis toxin or those expressing herbicide resistance such as glyphosate acetyltransferase, which provides resistance to glyphosate. Seed treatments with compounds of this invention can also increase vigor of plants growing from the seed.

Compounds of this invention and their compositions, both alone and in combination with other fungicides, nematicides and insecticides, are particularly useful in seed treatment for crops including, but not limited to, maize or corn, soybeans, cotton, cereal (e.g., wheat, oats, barley, rye and rice), potatoes, vegetables and oilseed rape.

Furthermore, the compounds of this invention are useful in treating postharvest diseases of fruits and vegetables caused by fungi and bacteria. These infections can occur before, during and after harvest. For example, infections can occur before harvest and then remain dormant until some point during ripening (e.g., host begins tissue changes in such a way that infection can progress); also infections can arise from surface wounds created by mechanical or insect injury. In this respect, the compounds of this invention can reduce losses (i.e. losses resulting from quantity and quality) due to postharvest diseases which may occur at any time from harvest to consumption. Treatment of postharvest diseases with compounds of the invention can increase the period of time during which perishable edible plant parts (e.g, fruits, seeds, foliage, stems, bulbs, tubers) can be stored refrigerated or un-refrigerated after harvest, and remain edible and free from noticeable or harmful degradation or contamination by fungi or other microorganisms. Treatment of edible plant parts before or after harvest with compounds of the invention can also decrease the formation of toxic metabolites of fungi or other microorganisms, for example, mycotoxins such as aflatoxins.

Plant disease control is ordinarily accomplished by applying an effective amount of a compound of this invention either pre- or post-infection, to the portion of the plant to be protected such as the roots, stems, foliage, fruits, seeds, tubers or bulbs, or to the media (soil or sand) in which the plants to be protected are growing. The compounds can also be applied to seeds to protect the seeds and seedlings developing from the seeds. The compounds can also be applied through irrigation water to treat plants. Control of postharvest pathogens which infect the produce before harvest is typically accomplished by field application of a compound of this invention, and in cases where infection occurs after harvest the compounds can be applied to the harvested crop as dips, sprays, fumigants, treated wraps and box liners.

Rates of application for these compounds (i.e. a fungicidally effective amount) can be influenced by factors such as the plant diseases to be controlled, the plant species to be protected, ambient moisture and temperature and should be determined under actual use conditions. One skilled in the art can easily determine through simple experimentation the fungicidally effective amount necessary for the desired level of plant disease control. Foliage can normally be protected when treated at a rate of from less than about 1 g/ha to about 5,000 g/ha of active ingredient. Seed and seedlings can normally be protected when seed is treated at a rate of from about 0.001 g (more typically about 0.1 g) to about 10 g per kilogram of seed.

Compounds of this invention can also be mixed with one or more other biologically active compounds or agents including fungicides, insecticides, nematocides, bactericides, acaricides, herbicides, herbicide safeners, growth regulators such as insect molting inhibitors and rooting stimulants, chemosterilants, semiochemicals, repellents, attractants, pheromones, feeding stimulants, plant nutrients, other biologically active compounds or entomopathogenic bacteria, virus or fungi to form a multi-component pesticide giving an even broader spectrum of agricultural protection. Thus the present invention also pertains to a composition comprising a compound of Formula 1 (in a fungicidally effective amount) and at least one additional biologically active compound or agent (in a biologically effective amount) and can further comprise at least one of a surfactant, a solid diluent or a liquid diluent. The other biologically active compounds or agents can be formulated in compositions comprising at least one of a surfactant, solid or liquid diluent. For mixtures of the present invention, one or more other biologically active compounds or agents can be formulated together with a compound of Formula 1, to form a premix, or one or more other biologically active compounds or agents can be formulated separately from the compound of Formula 1, and the formulations combined together before application (e.g., in a spray tank) or, alternatively, applied in succession.

As mentioned in the Summary of the Invention, one aspect of the present invention is a fungicidal composition comprising (i.e. a mixture or combination of) a compound of Formula 1, an N-oxide, or a salt thereof (i.e. component a), and at least one other fungicide (i.e. component b). Of note is such a combination where the other fungicidal active ingredient has different site of action from the compound of Formula 1. In certain instances, a combination with at least one other fungicidal active ingredient having a similar spectrum of control but a different site of action will be particularly advantageous for resistance management. Thus, a composition of the present invention can further comprise a fungicidally effective amount of at least one additional fungicidal active ingredient having a similar spectrum of control but a different site of action.

Of note is a composition which in addition to the Formula 1 compound of component (a), includes as component (b) at least one fungicidal compound selected from the group consisting of the FRAC-defined mode of action (MOA) classes (A) nucleic acid synthesis, (B) mitosis and cell division, (C) respiration, (D) amino acid and protein synthesis, (E) signal transduction, (F) lipid synthesis and membrane integrity, (G) sterol biosynthesis in membranes, (H) cell wall biosynthesis in membranes, (I) melanin synthesis in cell wall, (P) host plant defense induction, multi-site contact activity and unknown mode of action.

FRAC-recognized or proposed target sites of action along with their FRAC target site codes belonging to the above MOA classes are (A1) RNA polymerase I, (A2) adenosine deaminase, (A3) DNA/RNA synthesis (proposed), (A4) DNA topoisomerase, (B1-B3) β-tubulin assembly in mitosis, (B4) cell division (proposed), (B5) delocalization of spectrin-like proteins, (C1) complex I NADH odxido-reductase, (C2) complex II: succinate dehydrogenase, (C3) complex III: cytochrome bc1 (ubiquinol oxidase) at Qo site, (C4) complex III: cytochrome bc1 (ubiquinone reductase) at Qi site, (C5) uncouplers of oxidative phosphorylation, (C6) inhibitors of oxidative phosphorylation, ATP synthase, (C7) ATP production (proposed), (C8) complex III: cytochrome bc1 (ubiquinone reductase) at Qx (unknown) site, (D1) methionine biosynthesis (proposed), (D2-D5) protein synthesis, (E1) signal transduction (mechanism unknown), (E2-E3) MAP/histidine kinase in osmotic signal transduction, (F2) phospholipid biosynthesis, methyl transferase, (F3) lipid peroxidation (proposed), (F4) cell membrane permeability, fatty acids (proposed), (F6) microbial disrupters of pathogen cell membranes, (F7) cell membrane disruption (proposed), (G1) C14-demethylase in sterol biosynthesis, (G2) Δ14-reductase and Δ8→Δ7-isomerase in sterol biosynthesis, (G3) 3-keto reductase, C4-demethylation, (G4) squalene epoxidase in sterol biosynthesis, (H3) trehalase and inositol biosynthesis, (H4) chitin synthase, (H5) cellulose synthase, (I1) reductase in melanin biosynthesis and (12) dehydratase in melanin biosynthesis.

Of particular note is a composition which in addition to the Formula 1 compound of component (a), includes as component (b) at least one fungicidal compound selected from the group consisting of the classes (b1) methyl benzimidazole carbamate (MBC) fungicides; (b2) dicarboximide fungicides; (b3) demethylation inhibitor (DMI) fungicides; (b4) phenylamide fungicides; (b5) amine/morpholine fungicides; (b6) phospholipid biosynthesis inhibitor fungicides; (b7) succinate dehydrogenase inhibitor fungicides; (b8) hydroxy(2-amino-)pyrimidine fungicides; (b9) anilinopyrimidine fungicides; (b10) N-phenyl carbamate fungicides; (b11) quinone outside inhibitor (QoI) fungicides; (b12) phenylpyrrole fungicides; (b13) azanaphthalene fungicides; (b14) lipid peroxidation inhibitor fungicides; (b15) melanin biosynthesis inhibitor-reductase (MBI-R) fungicides; (b16) melanin biosynthesis inhibitor-dehydratase (MBI-D) fungicides; (b17) sterol biosynthesis inhibitor (SBI): Class III fungicides; (b18) squalene-epoxidase inhibitor fungicides; (b19) polyoxin fungicides; (b20) phenylurea fungicides; (b21) quinone inside inhibitor (QiI) fungicides; (b22) benzamide and thiazole carboxamide fungicides; (b23) enopyranuronic acid antibiotic fungicides; (b24) hexopyranosyl antibiotic fungicides; (b25) glucopyranosyl antibiotic: protein synthesis fungicides; (b26) glucopyranosyl antibiotic: trehalase and inositol biosynthesis fungicides; (b27) cyanoacetamideoxime fungicides; (b28) carbamate fungicides; (b29) oxidative phosphorylation uncoupling fungicides; (b30) organo tin fungicides; (b31) carboxylic acid fungicides; (b32) heteroaromatic fungicides; (b33) phosphonate fungicides; (b34) phthalamic acid fungicides; (b35) benzotriazine fungicides; (b36) benzene-sulfonamide fungicides; (b37) pyridazinone fungicides; (b38) thiophene-carboxamide fungicides; (b39) complex I NADH oxidoreductase inhibitor fungicides; (b40) carboxylic acid amide (CAA) fungicides; (b41) tetracycline antibiotic fungicides; (b42) thiocarbamate fungicides; (b43) benzamide fungicides; (b44) microbial fungicides; (b45) Q_{X}I fungicides; (b46) plant extract fungicides; (b47) host plant defense induction fungicides; (b48) multi-site contact activity fungicides; (b49) fungicides other than fungicides of classes (b1) through (b48); and salts of compounds of classes (b1) through (b48).

Further descriptions of these classes of fungicidal compounds are provided below.

(b1) “Methyl benzimidazole carbamate (MBC) fungicides” (FRAC code 1) inhibit mitosis by binding to β-tubulin during microtubule assembly. Inhibition of microtubule assembly can disrupt cell division, transport within the cell and cell structure. Methyl benzimidazole carbamate fungicides include benzimidazole and thiophanate fungicides. The benzimidazoles include benomyl, carbendazim, fuberidazole and thiabendazole. The thiophanates include thiophanate and thiophanate-methyl.

(b2) “Dicarboximide fungicides” (FRAC code 2) inhibit a MAP/histidine kinase in osmotic signal transduction. Examples include chlozolinate, iprodione, procymidone and vinclozolin.

(b3) “Demethylation inhibitor (DMI) fungicides” (FRAC code 3) (Sterol Biosynthesis Inhibitors (SBI): Class I) inhibit C14-demethylase, which plays a role in sterol production. Sterols, such as ergosterol, are needed for membrane structure and function, making them essential for the development of functional cell walls. Therefore, exposure to these fungicides results in abnormal growth and eventually death of sensitive fungi. DMI fungicides are divided between several chemical classes: azoles (including triazoles and imidazoles), pyrimidines, piperazines, pyridines and triazolinthiones. The triazoles include azaconazole, bitertanol, bromuconazole, cyproconazole, difenoconazole, diniconazole (including diniconazole-M), epoxiconazole, etaconazole, fenbuconazole, fluquinconazole, flusilazole, flutriafol, hexaconazole, imibenconazole, ipconazole, metconazole, myclobutanil, penconazole, propiconazole, quinconazole, simeconazole, tebuconazole, tetraconazole, triadimefon, triadimenol, triticonazole, uniconazole, uniconazole-P, α(1-chlorocyclopropyl)-α-[2-(2,2-dichiorocyclopropyl)ethyl]-1H-1,2,4-triazole-1-ethanol, rel-1-[[(2R,3S)-3-(2-chlorophenyl)-2-(2,4-difluorophenyl)-2-oxiranyl]methyl]-1H-1,2,4-triazole, rel-2-[[(2R,3S)-3-(2-chlorophenyl)-2-(2,4-difluorophenyl)-2-oxiranyl]methyl]-1,2-dihydro-3H-1,2,4-triazole-3-thione, and rel-1-[[(2R,3S)-3-(2-chlorophenyl)-2-(2,4-difluorophenyl)-2-oxiranyl]methyl]-5-(2-propen-1-ylthio)-1H-1,2,4-triazole. The imidazoles include econazole, imazalil, oxpoconazole, prochloraz, pefurazoate and triflumizole. The pyrimidines include fenarimol, nuarimol and triarimol. The piperazines include triforine. The pyridines include buthiobate, pyrifenox, pyrisoxazole (3-[(3R)-5-(4-chlorophenyl)-2,3-dimethyl-3-isoxazolidinyl]pyridine, mixture of 3R,5R- and 3R,5S-isomers) and (αS)-[3-(4-chloro-2-fluorophenyl)-5-(2,4-difluorophenyl)-4-isoxazolyl]-3-pyridinemethanol. The triazolinthiones include prothioconazole and 2-[2-(1-chlorocyclopropyl)-4-(2,2-dichlorocyclopropyl)-2-hydroxybutyl]-1,2-dihydro-3H-1,2,4-triazole-3-thione. Biochemical investigations have shown that all of the above mentioned fungicides are DMI fungicides as described by K. H. Kuck et al. in Modern Selective Fungicides—Properties, Applications and Mechanisms of Action, H. Lyr (Ed.), Gustav Fischer Verlag: New York, 1995, 205-258.

(b4) “Phenylamide fungicides” (FRAC code 4) are specific inhibitors of RNA polymerase in Oomycete fungi. Sensitive fungi exposed to these fungicides show a reduced capacity to incorporate uridine into rRNA. Growth and development in sensitive fungi is prevented by exposure to this class of fungicide. Phenylamide fungicides include acylalanine, oxazolidinone and butyrolactone fungicides. The acylalanines include benalaxyl, benalaxyl-M (also known as kiralaxyl), furalaxyl, metalaxyl and metalaxyl-M (also known as mefenoxam). The oxazolidinones include oxadixyl. The butyrolactones include ofurace.

(b5) “Amine/morpholine fungicides” (FRAC code 5) (SBI: Class II) inhibit two target sites within the sterol biosynthetic pathway, Δ⁸→Δ⁷ isomerase and Δ¹⁴ reductase. Sterols, such as ergosterol, are needed for membrane structure and function, making them essential for the development of functional cell walls. Therefore, exposure to these fungicides results in abnormal growth and eventually death of sensitive fungi. Amine/morpholine fungicides (also known as non-DMI sterol biosynthesis inhibitors) include morpholine, piperidine and spiroketal-amine fungicides. The morpholines include aldimorph, dodemorph, fenpropimorph, tridemorph and trimorphamide. The piperidines include fenpropidin and piperalin. The spiroketal-amines include spiroxamine.

(b6) “Phospholipid biosynthesis inhibitor fungicides” (FRAC code 6) inhibit growth of fungi by affecting phospholipid biosynthesis. Phospholipid biosynthesis fungicides include phophorothiolate and dithiolane fungicides. The phosphorothiolates include edifenphos, iprobenfos and pyrazophos. The dithiolanes include isoprothiolane.

(b7) “Succinate dehydrogenase inhibitor (SDHI) fungicides”” (FRAC code 7) inhibit Complex II fungal respiration by disrupting a key enzyme in the Krebs Cycle (TCA cycle) named succinate dehydrogenase. Inhibiting respiration prevents the fungus from making ATP, and thus inhibits growth and reproduction. SDHI fungicides include phenylbenzamide, furan carboxamide, oxathiin carboxamide, thiazole carboxamide, pyrazole-4-carboxamide, pyridine carboxamide, phenyl oxoethyl thiophene amides and pyridinylethyl benzamides. The benzamides include benodanil, flutolanil and mepronil. The furan carboxamides include fenfuram. The oxathiin carboxamides include carboxin and oxycarboxin. The thiazole carboxamides include thifluzamide. The pyrazole-4-carboxamides include benzovindiflupyr (N-[9-(dichloromethylene)-1,2,3,4-tetrahydro-1,4-methanonaphthalen-5-yl]-3-(difluoromethyl)-1-methyl-1H-pyrazole-4-carboxamide), bixafen, fluxapyroxad (3-(difluoromethyl)-1-methyl-N-(3′,4′,5′-trifluoro[1,1′-biphenyl]-2-yl)-1H-pyrazole-4-carboxamide), furametpyr, isopyrazam (3-(difluoromethyl)-1-methyl-N-[1,2,3,4-tetrahydro-9-(1-methylethy 1)-1,4-methanonaphthalen-5-yl]-1H-pyrazole-4-carboxamide), penflufen (N-[2-(1,3-dimethylbutyl)phenyl]-5-fluoro-1,3-dimethyl-1H-pyrazole-4-carboxamide), penthiopyrad, sedaxane (N-[2-[1,1′-bicyclopropyl]-2-ylphenyl]-3-(difluoromethyl)-1-methyl-1H-pyrazole-4-carboxamide), N-[2-(1S,2R)-[1,1′-bicyclopropyl]-2-ylphenyl]-3-(difluoromethyl)-1-methyl-1H-pyrazole-4-carboxamide, 3-(difluoromethyl)-N-(2,3-dihydro-1,1,3-trimethyl-1H-inden-4-yl)-1-methyl-1H-pyrazole-4-carboxamide, N-[2-(2,4-dichlorophenyl)-2-methoxy-1-methylethyl]-3-(difluoromethyl)-1-methyl-1H-pyrazole-4-carboxamide and N-cyclopropyl-3-(difluoromethyl)-5-fluoro-1-methyl-N-[[2-(1-methyl-ethyl)phenyl]methyl]-1H-pyrazole-4-carboxamide. The pyridine carboxamides include boscalid. The phenyl oxoethyl thiophene amides include isofetamid (N-[1,1-dimethyl-2-[2-methyl-4-(1-methylethoxy)phenyl]-2-oxoethyl]-3-methyl-2-thiophenecarboxamide). The pyridinylethyl benzamides include fluopyram.

(b8) “Hydroxy-(2-amino-)pyrimidine fungicides” (FRAC code 8) inhibit nucleic acid synthesis by interfering with adenosine deaminase. Examples include bupirimate, dimethirimol and ethirimol.

(b9) “Anilinopyrimidine fungicides” (FRAC code 9) are proposed to inhibit biosynthesis of the amino acid methionine and to disrupt the secretion of hydrolytic enzymes that lyse plant cells during infection. Examples include cyprodinil, mepanipyrim and pyrimethanil.

(b10) “N-Phenyl carbamate fungicides” (FRAC code 10) inhibit mitosis by binding to β-tubulin and disrupting microtubule assembly. Inhibition of microtubule assembly can disrupt cell division, transport within the cell and cell structure. Examples include diethofencarb.

(b11) “Quinone outside inhibitor (QoI) fungicides” (FRAC code 11) inhibit Complex III mitochondrial respiration in fungi by affecting ubiquinol oxidase. Oxidation of ubiquinol is blocked at the “quinone outside” (Q_{O}) site of the cytochrome bc₁ complex, which is located in the inner mitochondrial membrane of fungi. Inhibiting mitochondrial respiration prevents normal fungal growth and development. Quinone outside inhibitor fungicides include methoxyacrylate, methoxycarbamate, oximinoacetate, oximinoacetamide and dihydrodioxazine fungicides (collectively also known as strobilurin fungicides), and oxazolidinedione, imidazolinone and benzylcarbamate fungicides. The methoxyacrylates include azoxystrobin, coumoxystrobin (methyl (αE)-2-[[(3-butyl-4-methyl-2-oxo-2H-1-benzopyran-7-yl)oxy]methyl]-α-(methoxymethylene)benzeneacetate), enoxastrobin (methyl (αE)-2-[[[(E)-[(2E)-3-(4-chlorophenyl)-1-methyl-2-propen-1-ylidene]amino]oxy]methyl]-α-(methoxymethylene)benzeneaceate) (also known as enestroburin), flufenoxystrobin (methyl (αE)-2-[[2-chloro-4-(trifluoromethyl)phenoxy]methyl]-α-(methoxymethylene)benzeneacetate), picoxystrobin, and pyraoxystrobin (methyl (αE)-2-[[[3-(4-chlorophenyl)-1-methyl-1H-pyrazol-5-yl]oxy]methyl]-α-(methoxymethylene)benzeneacetate). The methoxycarbamates include pyraclostrobin, pyrametostrobin (methyl N-[2-[[(1,4-dimethyl-3-phenyl-1H-pyrazol-5-yl)oxy]methyl]phenyl]-N-methoxycarbamate) and triclopyricarb (methyl N-methoxy-N-[2-[[(3,5,6-trichloro-2-pyridinyl)oxy]methyl]phenyl]carbamate). The oximinoacetates include kresoxim-methyl, and trifloxystrobin. The oximinoacetamides include dimoxystrobin, fenaminstrobin ((αE)-2-[[[(E)-[(2E)-3-(2,6-dichlorophenyl)-1-methyl-2-propen-1-ylidene]amino]oxy]methyl]-α-(methoxyimino)-N-methylbenzeneacetamide), metominostrobin, orysastrobin and α-[methoxyimino]-N-methyl-2-[[[1-[3-(trifluoro-methyl)phenyl]ethoxy]imino]methyl]benzeneacetamide. The dihydrodioxazines include fluoxastrobin. The oxazolidinediones include famoxadone. The imidazolinones include fenamidone. The benzylcarbamates include pyribencarb. Class (b11) also includes mandestrobin (2-[(2, 5-dimethylphenoxy)methyl]-α-methoxy-N-benzeneacetamide).

(b12) “Phenylpyrrole fungicides” (FRAC code 12) inhibit a MAP/histidine kinase associated with osmotic signal transduction in fungi. Fenpiclonil and fludioxonil are examples of this fungicide class.

(b13) “Azanaphthalene fungicides” (FRAC code 13) are proposed to inhibit signal transduction by a mechanism which is as yet unknown. They have been shown to interfere with germination and/or appressorium formation in fungi that cause powdery mildew diseases. Azanaphthalene fungicides include aryloxyquinolines and quinazolinones. The aryloxyquinolines include quinoxyfen. The quinazolinones include proquinazid.

(b14) “Lipid peroxidation inhibitor fungicides” (FRAC code 14) are proposed to inhibit lipid peroxidation which affects membrane synthesis in fungi. Members of this class, such as etridiazole, may also affect other biological processes such as respiration and melanin biosynthesis. Lipid peroxidation fungicides include aromatic hydrocarbon and 1,2,4-thiadiazole fungicides. The aromatic hydrocarboncarbon fungicides include biphenyl, chloroneb, dicloran, quintozene, tecnazene and tolclofos-methyl. The 1,2,4-thiadiazoles include etridiazole.

(b15) “Melanin biosynthesis inhibitors-reductase (MBI-R) fungicides” (FRAC code 16.1) inhibit the naphthal reduction step in melanin biosynthesis. Melanin is required for host plant infection by some fungi. Melanin biosynthesis inhibitors-reductase fungicides include isobenzofuranone, pyrroloquinolinone and triazolobenzothiazole fungicides. The isobenzofuranones include fthalide. The pyrroloquinolinones include pyroquilon. The triazolobenzothiazoles include tricyclazole.

(b16) “Melanin biosynthesis inhibitors-dehydratase (MBI-D) fungicides” (FRAC code 16.2) inhibit scytalone dehydratase in melanin biosynthesis. Melanin in required for host plant infection by some fungi. Melanin biosynthesis inhibitors-dehydratase fungicides include cyclopropanecarboxamide, carboxamide and propionamide fungicides. The cyclopropanecarboxamides include carpropamid. The carboxamides include diclocymet. The propionamides include fenoxanil.

(b17) “Sterol Biosynthesis Inhibitor (SBI): Class III fungicides (FRAC code 17) inhibit 3-ketoreductase during C4-demethylation in sterol production. SBI: Class III inhibitors include hydroxyanilide fungicides and amino-pyrazolinone fungicides. Hydroxyanilides include fenhexamid. Amino-pyrazolinones include fenpyrazamine (S-2-propen-1-yl 5-amino-2,3-dihydro-2-(1-methylethyl)-4-(2-methylphenyl)-3-oxo-1H-pyrazole-1-carbothioate).

(b18) “Squalene-epoxidase inhibitor fungicides” (FRAC code 18) (SBI: Class IV) inhibit squalene-epoxidase in the sterol biosynthesis pathway. Sterols such as ergosterol are needed for membrane structure and function, making them essential for the development of functional cell walls. Therefore exposure to these fungicides results in abnormal growth and eventually death of sensitive fungi. Squalene-epoxidase inhibitor fungicides include thiocarbamate and allylamine fungicides. The thiocarbamates include pyributicarb. The allylamines include naftifine and terbinafine.

(b19) “Polyoxin fungicides” (FRAC code 19) inhibit chitin synthase. Examples include polyoxin.

(b20) “Phenylurea fungicides” (FRAC code 20) are proposed to affect cell division. Examples include pencycuron.

(b21) “Quinone inside inhibitor (QilI) fungicides” (FRAC code 21) inhibit Complex III mitochondrial respiration in fungi by affecting ubiquinone reductase. Reduction of ubiquinone is blocked at the “quinone inside” (Qi) site of the cytochrome bc₁ complex, which is located in the inner mitochondrial membrane of fungi. Inhibiting mitochondrial respiration prevents normal fungal growth and development. Quinone inside inhibitor fungicides include cyanoimidazole and sulfamoyltriazole fungicides. The cyanoimidazoles include cyazofamid. The sulfamoyltriazoles include amisulbrom.

(b22) “Benzamide and thiazole carboxamide fungicides” (FRAC code 22) inhibit mitosis by binding to β-tubulin and disrupting microtubule assembly. Inhibition of microtubule assembly can disrupt cell division, transport within the cell and cell structure. The benzamides include zoxamide. The thiazole carboxamides include ethaboxam.

(b23) “Enopyranuronic acid antibiotic fungicides” (FRAC code 23) inhibit growth of fungi by affecting protein biosynthesis. Examples include blasticidin-S.

(b24) “Hexopyranosyl antibiotic fungicides” (FRAC code 24) inhibit growth of fungi by affecting protein biosynthesis. Examples include kasugamycin.

(b25) “Glucopyranosyl antibiotic: protein synthesis fungicides” (FRAC code 25) inhibit growth of fungi by affecting protein biosynthesis. Examples include streptomycin.

(b26) “Glucopyranosyl antibiotic: trehalase and inositol biosynthesis fungicides” (FRAC code 26) inhibit trehalase and inositol biosynthesis. Examples include validamycin.

(b27) “Cyanoacetamideoxime fungicides (FRAC code 27) include cymoxanil.

(b28) “Carbamate fungicides” (FRAC code 28) are considered multi-site inhibitors of fungal growth. They are proposed to interfere with the synthesis of fatty acids in cell membranes, which then disrupts cell membrane permeability. Propamacarb, iodocarb, and prothiocarb are examples of this fungicide class.

(b29) “Oxidative phosphorylation uncoupling fungicides” (FRAC code 29) inhibit fungal respiration by uncoupling oxidative phosphorylation. Inhibiting respiration prevents normal fungal growth and development. This class includes 2,6-dinitroanilines such as fluazinam, and dinitrophenyl crotonates such as dinocap, meptyldinocap and binapacryl.

(b30) “Organo tin fungicides” (FRAC code 30) inhibit adenosine triphosphate (ATP) synthase in oxidative phosphorylation pathway. Examples include fentin acetate, fentin chloride and fentin hydroxide.

(b31) “Carboxylic acid fungicides” (FRAC code 31) inhibit growth of fungi by affecting deoxyribonucleic acid (DNA) topoisomerase type II (gyrase). Examples include oxolinic acid.

(b32) “Heteroaromatic fungicides” (Fungicide Resistance Action Committee (FRAC) code 32) are proposed to affect DNA/ribonucleic acid (RNA) synthesis. Heteroaromatic fungicides include isoxazoles and isothiazolones. The isoxazoles include hymexazole and the isothiazolones include octhilinone.

(b33) “Phosphonate fungicides” (FRAC code 33) include phosphorous acid and its various salts, including fosetyl-aluminum.

(b34) “Phthalamic acid fungicides” (FRAC code 34) include teclofthalam.

(b35) “Benzotriazine fungicides” (FRAC code 35) include triazoxide.

(b36) “Benzene-sulfonamide fungicides” (FRAC code 36) include flusulfamide.

(b37) “Pyridazinone fungicides” (FRAC code 37) include diclomezine.

(b38) “Thiophene-carboxamide fungicides” (FRAC code 38) are proposed to affect ATP production. Examples include silthiofam.

(b39) “Complex I NADH oxidoreductase inhibitor fungicides” (FRAC code 39) inhibit electron transport in mitochondria and include pyrimidinamines such as diflumetorim, and pyrazole-5-carboxamides such as tolfenpyrad.

(b40) “Carboxylic acid amide (CAA) fungicides” (FRAC code 40) inhibit cellulose synthase which prevents growth and leads to death of the target fungus. Carboxylic acid amide fungicides include cinnamic acid amide, valinamide and other carbamate, and mandelic acid amide fungicides. The cinnamic acid amides include dimethomorph, flumorph and pyrimorph (3-(2-chloro-4-pyridinyl)-3-[4-(1,1-dimethylethyl)phenyl]-1-(4-morpholinyl)-2-propene-1-one). The valinamide and other carbamates include benthiavalicarb, benthiavalicarb-isopropyl, iprovalicarb, tolprocarb (2,2,2-trifluoroethyl N-[(1S)-2-methyl-1-[[(4-methylbenzoyl)amino]methyl]propyl]carbamate) and valifenalate (methyl N-[(1-methylethoxy)carbonyl]-L-valyl-3-(4-chlorophenyl)-3-alaninate) (also known as valiphenal). The mandelic acid amides include mandipropamid, N-[2-[4-[[3-(4-chlorophenyl)-2-propyn-1-yl]oxy]-3-methoxyphenyl]ethyl]-3-methyl-2-[(methylsulfonyl)-amino]butanamide and N-[2-[4-[[3-(4-chlorophenyl)-2-propyn-1-yl]oxy]-3-methoxyphenyl]-ethyl]-3-methyl-2-[(ethyl sulfonyl)amino]butanamide.

(b41) “Tetracycline antibiotic fungicides” (FRAC code 41) inhibit growth of fungi by affecting protein synthesis. Examples include oxytetracycline.

(b42) “Thiocarbamate fungicides” (FRAC code 42) include methasulfocarb.

(b43) “Benzamide fungicides” (FRAC code 43) inhibit growth of fungi by delocalization of spectrin-like proteins. Examples include pyridinylmethyl benzamide fungicides such as fluopicolide (now FRAC code 7, pyridinylethyl benzamides).

(b44) “Microbial fungicides” (FRAC code 44) disrupt fungal pathogen cell membranes. Microbial fungicides include Bacillus species such as Bacillus amyloliquefaciens strains QST 713, FZB24, MB1600, D747 and the fungicidal lipopeptides which they produce.

(b45) “Q_{X}I fungicides” (FRAC code 45) inhibit Complex III mitochondrial respiration in fungi by affecting ubiquinone reductase at an unknown (Qx) site of the cytochrome bc₁ complex. Inhibiting mitochondrial respiration prevents normal fungal growth and development. Q_{X}I fungicides include triazolopyrimidylamines such as ametoctradin (5-ethyl-6-octyl[1,2,4]triazolo[1,5-a]pyrimidin-7-amine).

(b46) “Plant extract fungicides” are proposed to act by cell membrane disruption. Plant extract fungicides include terpene hydrocarbons and terpene alcohols such as the extract from Melaleuca alternifolia (tea tree).

(b47) “Host plant defense induction fungicides” (FRAC code P) induce host plant defense mechanisms. Host plant defense induction fungicides include benzothiadiazoles, benzisothiazole and thiadiazole-carboxamide fungicides. The benzothiadiazoles include acibenzolar-S-methyl. The benzisothiazoles include probenazole. The thiadiazole-carboxamides include tiadinil and isotianil.

(b48) “Multi-site contact fungicides” inhibit fungal growth through multiple sites of action and have contact/preventive activity. This class of fungicides includes: (b48.1) “copper fungicides” (FRAC code M1)”, (b48.2) “sulfur fungicides” (FRAC code M2), (b48.3) “dithiocarbamate fungicides” (FRAC code M3), (b48.4) “phthalimide fungicides” (FRAC code M4), (b48.5) “chloronitrile fungicides” (FRAC code M5), (b48.6) “sulfamide fungicides” (FRAC code M6), (b48.7) multi-site contact “guanidine fungicides” (FRAC code M7), (b48.8) “triazine fungicides” (FRAC code M8), (b48.9) “quinone fungicides” (FRAC code M9), (b48.10) “quinoxaline fungicides” (FRAC code M10) and (b48.11) “maleimide fungicides” (FRAC code M11). “Copper fungicides” are inorganic compounds containing copper, typically in the copper(II) oxidation state; examples include copper oxychloride, copper sulfate and copper hydroxide, including compositions such as Bordeaux mixture (tribasic copper sulfate). “Sulfur fungicides” are inorganic chemicals containing rings or chains of sulfur atoms; examples include elemental sulfur. “Dithiocarbamate fungicides” contain a dithiocarbamate molecular moiety; examples include mancozeb, metiram, propineb, ferbam, maneb, thiram, zineb and ziram. “Phthalimide fungicides” contain a phthalimide molecular moiety; examples include folpet, captan and captafol. “Chloronitrile fungicides” contain an aromatic ring substituted with chloro and cyano; examples include chlorothalonil. “Sulfamide fungicides” include dichlofluanid and tolyfluanid. Multi-site contact “guanidine fungicides” include, guazatine, iminoctadine albesilate and iminoctadine triacetate. “Triazine fungicides” include anilazine. “Quinone fungicides” include dithianon. “Quinoxaline fungicides” include quinomethionate (also known as chinomethionate). “Maleimide fungicides” include fluoroimide.

(b49) “Fungicides other than fungicides of classes (b1) through (b48)” include certain fungicides whose mode of action may be unknown. These include: (b49.1), “phenyl-acetamide fungicides” (FRAC code U6), (b49.2) “aryl-phenyl-ketone fungicides” (FRAC code U8), (b49.3) “guanidine fungicides” (FRAC code U12), (b49.4) “thiazolidine fungicides” (FRAC code U13), (b49.5) “pyrimidinone-hydrazone fungicides” (FRAC code U14) and (b49.6) compounds that bind to oxysterol-binding protein as described in PCT Patent Publication WO 2013/009971. The phenyl-acetamides include cyflufenamid and N-[[(cyclopropylmethoxy)amino][6-(difluoromethoxy)-2,3-difluorophenyl]-methylene]-benzeneacetamide. The aryl-phenyl ketones include benzophenones such as metrafenone, and benzoylpyridines such as pyriofenone (5-chloro-2-methoxy-4-methyl-3-pyridinyl)(2,3,4-trimethoxy-6-methylphenyl)methanone). The quanidines include dodine. The thiazolidines include flutianil ((2Z)-2-[[2-fluoro-5-(trifluoromethyl)phenyl]thio]-2-[3-(2-methoxyphenyl)-2-thiazolidinylidene]acetonitrile). The pyrimidinonehydrazones include ferimzone. The (b49.6) class includes oxathiapiprolin (1-[4-[4-[5-(2,6-difluorophenyl)-4,5-dihydro-3-isoxazolyl]-2-thiazolyl]-1-piperidinyl]-2-[5-methyl-3-(trifluoromethyl)-1H-pyrazol-1-yl]ethanone) and its R-enantiomer which is 1-[4-[4-[5R-(2,6-difluorophenyl)-4,5-dihydro-3-isoxazolyl]-2-thiazolyl]-1-piperidinyl]-2-[5-methyl-3-(trifluoromethyl)-1H-pyrazol-1-yl]-ethanone (Registry Number 1003319-79-6).

The (b49) class also includes bethoxazin, flometoquin (2-ethyl-3,7-dimethyl-6-[4-(trifluoromethoxy)phenoxy]-4-quinolinyl methyl carbonate), fluoroimide, neo-asozin (ferric methanearsonate), picarbutrazox (1,1-dimethylethyl N-[6-[[[[((Z)-1-methyl-1H-tetrazol-5-yl)phenylmethylene]amino]oxy]methyl]-2-pyridinyl]carbamate), pyrrolnitrin, quinomethionate, tebufloquin (6-(1,1-dimethylethyl)-8-fluoro-2,3-dimethyl-4-quinolinyl acetate), tolnifanide (N-(4-chloro-2-nitrophenyl)-N-ethyl-4-methylbenzenesulfonamide), 2-butoxy-6-iodo-3-propyl-4H-1-benzopyran-4-one, 3-butyn-1-yl N-[6-[[[[(1-methyl-1H-tetrazol-5-yl)phenylmethylene]amino]oxy]methyl]-2-pyridinyl]carbamate, (N-(4-chloro-2-nitrophenyl)-N-ethyl-4-methylbenzenesulfonamide), N-[4-[4-chloro-3-(trifluoromethyl)-phenoxy]-2, 5-dimethylphenyl]-N-ethyl-N-methylmethanimidamide, N-[[(cyclopropyl-methoxy)amino][6-(difluoromethoxy)-2,3-difluorophenyl]methylene]benzeneacetamide, 2,6-dimethyl-1H,5H-[1,4]dithiino[2,3-c:5,6-c′]dipyrrole-1,3,5,7(2H,6H)-tetrone, 5-fluoro-2-[(4-methylphenyl)methoxy]-4-pyrimidinamine, 5-fluoro-2-[(4-fluorophenyl)methoxy]-4-pyrimidinamine and 4-fluorophenyl N-[1-[[[1-(4-cyanophenyl)ethyl]sulfonyl]methyl]-propyl]carbamate, pentyl N-[6-[[[[(1-methyl-1H-tetrazol-5-yl)phenylmethylene]amino]oxy]methyl]-2-pyridinyl]carbamate, pentyl N-[4-[[[[(1-methyl-1H-tetrazol-5-yl)phenylmethylene]amino]oxy]methyl]-2-thiazolyl]carbamate and pentyl N-[6-[[[[(Z)-(1-methyl-1H-tetrazol-5-yl)phenylmethylene]amino]oxy]methyl]-2-pyridinyl]-carbamate. The (b46) class further includes mitosis- and cell division-inhibiting fungicides besides those of the particular classes described above (e.g., (b1), (b10) and (b22)).

Additional “Fungicides other than fungicides of classes (1) through (46)” whose mode of action may be unknown, or may not yet be classified include a fungicidal compound selected from components (b49.7) through (b49.12), as shown below.

Component (b49.7) relates to a compound of Formula b49.7

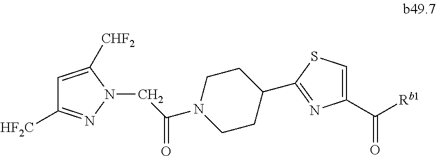

wherein R^{b1} is

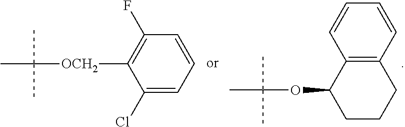

Examples of a compound of Formula b49.7 include (b49.7a) (2-chloro-6-fluorophenyl)-methyl 2-[1-[2-[3,5-bis(difluoromethyl)-1H-pyrazol-1-yl]acetyl]-4-piperidinyl]-4-thiazolecarboxylate (Registry Number 1299409-40-7) and (b49.7b) (1R)-1,2,3,4-tetrahydro-1-naphthalenyl 2-[1-[2-[3,5-bis(difluoromethyl)-1H-pyrazol-1-yl]acetyl]-4-piperidinyl]-4-thiazolecarboxylate (Registry Number 1299409-42-9). Methods for preparing compounds of Formula b46.2 are described in PCT Patent Publications WO 2009/132785 and WO 2011/051243.

Component (b49.8) relates to a compound of Formula b49.8

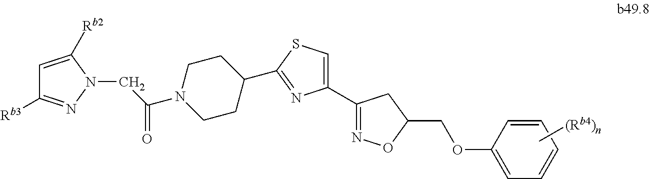

wherein R^{b2} is CH₃, CF₃ or CHF₂; R^{b3} is CH₃, CF₃ or CHF₂; R^{b4} is halogen or cyano; and n is 0, 1, 2 or 3.
   Examples of a compound of Formula b49.8 include (b49.8a) 1-[4-[4-[5-[(2,6-difluorophenoxy)methyl]-4,5-dihydro-3-isoxazolyl]-2-thiazolyl]-1-piperdinyl]-2-[5-methyl-3-(trifluoromethyl)-1H-pyrazol-1-yl]ethanone. Methods for preparing compounds of Formula b49.8 are described in PCT Patent Application PCT/US 11/64324.

Component (b4799) relates to a compound of Formula b49.9

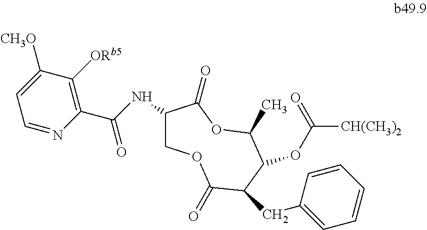

wherein R^{b5} is —CH₂OC(O)CH(CH₃)₂, —C(O)CH₃, —CH₂OC(O)CH₃, —C(O)OCH₂CH(CH₃)₂ or

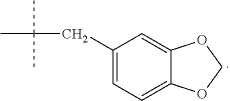

Examples of a compound of Formula b49.9 include (b49.9a) [[4-methoxy-2-[[[(3S,7R,8R,9S)-9-methyl-8-(2-methyl-1-oxopropoxy)-2,6-dioxo-7-(phenylmethyl)-1,5-dioxonan-3-yl]amino]carbonyl]-3-pyridinyl]oxy]methyl 2-methylpropanoate (Registry Number 517875-34-2), (b49.9b) (3S,6S,7R,8R)-3-[[[3-(acetyloxy)-4-methoxy-2-pyridinyl]-carbonyl]amino]-6-methyl-4,9-dioxo-8-(phenylmethyl)-1,5-dioxonan-7-yl 2-methylpropanoate (Registry Number 234112-93-7), (b49.9c) (3S,6S,7R,8R)-3-[[[3-[(acetyloxy)methoxy]-4-methoxy-2-pyridinyl]carbonyl]amino]-6-methyl-4,9-dioxo-8-(phenylmethyl)-1,5-dioxonan-7-yl 2-methylpropanoate (Registry Number 517875-31-9), (b49.9d) (3S,6S,7R,8R)-3-[[[4-methoxy-3-[[(2-methylpropoxy)carbonyl]oxy]-2-pyridinyl]-carbonyl]amino]-6-methyl-4,9-dioxo-8-(phenylmethyl)-1,5-dioxonan-7-yl 2-methylpropanoate (Registry Number 328256-72-0), and (b49.9e) N-[[3-(1,3-benzodioxol-5-ylmethoxy)-4-methoxy-2-pyridinyl]carbonyl]-O-[2, 5-dideoxy-3-O-(2-methyl-1-oxopropyl)-2-(phenylmethyl)-L-arabinonoyl]-L-serine, (1-4′)-lactone (Registry Number 1285706-70-8). Methods for preparing compounds of Formula b49.9 are described in PCT Patent Publications WO 99/40081, WO 2001/014339, WO 2003/035617 and WO 2011044213.

Component (b49.10) relates to a compound of Formula b49.10

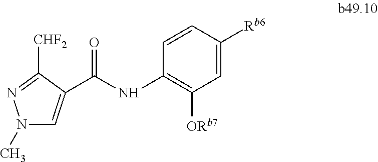

wherein R^{b6} is H or F, and R^{b7} is —CF₂CHFCF₃ or —CF₂CF₂H. Examples of a compound of Formula b49.10 are (b49.10a) 3-(difluoromethyl)-N-[4-fluoro-2-(1,1,2,3,3,3-hexafluoro-propoxy)phenyl]-1-methyl-1H-pyrazole-4-carboxamide (Registry Number 1172611-40-3) and (b49.10b) 3-(difluoromethyl)-1-methyl-N-[2-(1,1,2,2-tetrafluoroethoxy)phenyl]-1H-pyrazole-4-carboxamide (Registry Number 923953-98-4). Compounds of Formula 49.10 can be prepared by methods described in PCT Patent Publication WO 2007/017450.

Component b49.11 relates a compound of Formula b49.11

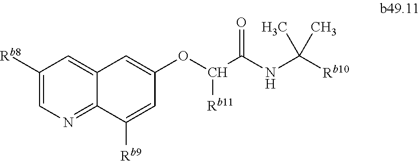

wherein
R^{b8} is halogen, C₁-C₄ alkoxy or C₂-C₄ alkynyl;
R^{b9} is H, halogen or C₁-C₄ alkyl;
R^{b10} is C₁-C₁₂ alkyl, C₁-C₁₂ haloalkyl, C₁-C₁₂ alkoxy, C₂-C₁₂ alkoxyalkyl, C₂-C₁₂ alkenyl, C₂-C₁₂ alkynyl, C₄-C₁₂ alkoxyalkenyl, C₄-C₁₂ alkoxyalkynyl, C₁-C₁₂ alkylthio or C₂-C₁₂ alkylthioalkyl;
R^{b11} is methyl or —Y^{b13}—R^{b12};
R^{b12} is C₁-C₂ alkyl; and
Y^{b13} is CH₂, O or S.
   Examples of compounds of Formula b49.11 include (b49.11a) 2-[(3-bromo-6-quinolinyl)oxy]-N-(1,1-dimethyl-2-butyn-1-yl)-2-(methylthio)acetamide, (b49.11b) 2-[(3-ethynyl-6-quinolinyl)oxy]-N-[1-(hydroxymethyl)-1-methyl-2-propyn-1-yl]-2-(methylthio)-acetamide, (b49.11c) N-(1,1-dimethyl-2-butyn-1-yl)-2-[(3-ethynyl-6-quinolinyl)oxy]-2-(methylthio)acetamide, (b49.11 d) 2-[(3-bromo-8-methyl-6-quinolinyl)oxy]-N-(1,1-dimethyl-2-propyn-1-yl)-2-(methylthio)acetamide and (b49.11e) 2-[(3-bromo-6-quinolinyl)oxy]-N-(1,1-dimethylethyl)butanamide. Compounds of Formula b49.11, their use as fungicides and methods of preparation are generally known; see, for example, PCT Patent Publications WO 2004/047538, WO 2004/108663, WO 2006/058699, WO 2006/058700, WO 2008/110355, WO 2009/030469, WO 2009/049716 and WO 2009/087098.

Component 49.12 relates to N-[4-[[3-[(4-chlorophenyl)methyl]-1,2,4-thiadiazol-5-yl]oxy]-2,5-dimethylphenyl]-N-ethyl-N-methylmethanimidamide, which is believed to inhibit C24-methyl transferase involved in the biosynthesis of sterols.

Therefore of note is a mixture (i.e. composition) comprising a compound of Formula 1 and at least one fungicidal compound selected from the group consisting of the aforedescribed classes (1) through (49). Also of note is a composition comprising said mixture (in fungicidally effective amount) and further comprising at least one additional component selected from the group consisting of surfactants, solid diluents and liquid diluents. Of particular note is a mixture (i.e. composition) comprising a compound of Formula 1 and at least one fungicidal compound selected from the group of specific compounds listed above in connection with classes (1) through (49). Also of particular note is a composition comprising said mixture (in fungicidally effective amount) and further comprising at least one additional surfactant selected from the group consisting of surfactants, solid diluents and liquid diluents.

Examples of component (b) fungicides include acibenzolar-S-methyl, aldimorph, ametoctradin, amisulbrom, anilazine, azaconazole, azoxystrobin, benalaxyl (including benalaxyl-M), benodanil, benomyl, benthiavalicarb (including benthiavalicarb-isopropyl), benzovindiflupyr, bethoxazin, binapacryl, biphenyl, bitertanol, bixafen, blasticidin-S, boscalid, bromuconazole, bupirimate, buthiobate, captafol, captan, carbendazim, carboxin, carpropamid, chloroneb, chlorothalonil, chlozolinate, clotrimazole, copper hydroxide, copper oxychloride, copper sulfate, coumoxystrobin, cyazofamid, cyflufenamid, cymoxanil, cyproconazole, cyprodinil, dichlofluanid, diclocymet, diclomezine, dicloran, diethofencarb, difenoconazole, diflumetorim, dimethirimol, dimethomorph, dimoxystrobin, diniconazole (including diniconazole-M), dinocap, dithianon, dithiolanes, dodemorph, dodine, econazole, edifenphos, enoxastrobin (also known as enestroburin), epoxiconazole, etaconazole, ethaboxam, ethirimol, etridiazole, famoxadone, fenamidone, fenarimol, fenaminstrobin, fenbuconazole, fenfuram, fenhexamid, fenoxanil, fenpiclonil, fenpropidin, fenpropimorph, fenpyrazamine, fentin acetate, fentin chloride, fentin hydroxide, ferbam, ferimzone, flometoquin, fluazinam, fludioxonil, flufenoxystrobin, flumorph, fluopicolide, fluopyram, flouroimide, fluoxastrobin, fluquinconazole, flusilazole, flusulfamide, flutianil, flutolanil, flutriafol, fluxapyroxad, folpet, fthalide, fuberidazole, furalaxyl, furametpyr, guazatine, hexaconazole, hymexazole, imazalil, imibenconazole, iminoctadine albesilate, iminoctadine triacetate, iodocarb, ipconazole, iprobenfos, iprodione, iprovalicarb, isoconazole, isofetamid, isoprothiolane, isopyrazam, isotianil, kasugamycin, kresoxim-methyl, mancozeb, mandepropamid, mandestrobin, maneb, mepanipyrim, mepronil, meptyldinocap, metalaxyl (including metalaxyl-M/mefenoxam), metconazole, methasulfocarb, metiram, metominostrobin, metrafenone, miconazole, myclobutanil, naftifine, neo-asozin, nuarimol, octhilinone, ofurace, orysastrobin, oxadixyl, oxathiapiprolin, oxolinic acid, oxpoconazole, oxycarboxin, oxytetracycline, pefurazoate, penconazole, pencycuron, penflufen, penthiopyrad, phosphorous acid (including salts thereof, e.g., fosetyl-aluminum), picarbutrazox, picoxystrobin, piperalin, polyoxin, probenazole, prochloraz, procymidone, propamacarb, propiconazole, propineb, proquinazid, prothiocarb, prothioconazole, pyraclostrobin, pyrametostrobin, pyraoxystrobin, pyrazophos, pyribencarb, pyributicarb, pyrifenox, pyrimethanil, pyriofenone, pyrisoxazole, pyroquilon, pyrrolnitrin, quinconazole, quinomethionate, quinoxyfen, quintozene, sedaxane, silthiofam, simeconazole, spiroxamine, streptomycin, sulfur, tebuconazole, tebufloquin, teclofthalam, tecnazene, terbinafine, tetraconazole, thiabendazole, thifluzamide, thiophanate, thiophanate-methyl, thiram, tiadinil, tolclofos-methyl, tolnifanide, tolprocarb, tolyfluanid, triadimefon, triadimenol, triarimol, triticonazole, triazoxide, tribasic copper sulfate, tricyclazole, triclopyricarb, tridemorph, trifloxystrobin, triflumizole, triforine, trimorphamide, uniconazole, uniconazole-P, validamycin, valifenalate (also known as valiphenal), vinclozolin, zineb, ziram, zoxamide, (3 S,6S,7R,8R)-3-[[[3-[(acetyloxy)methoxy]-4-methoxy-2-pyridinyl]carbonyl]amino]-6-methyl-4,9-dioxo-8-(phenylmethyl)-1,5-dioxonan-7-yl 2-methylpropanoate, (3S,6S,7R,8R)-3-[[[3-(acetyloxy)-4-methoxy-2-pyridinyl]carbonyl]amino]-6-methyl-4,9-dioxo-8-(phenylmethyl)-1,5-dioxonan-7-yl 2-methylpropanoate, N-[[3-(1,3-benzodioxol-5-ylmethoxy)-4-methoxy-2-pyridinyl]carbonyl]-O-[2,5-dideoxy-3-O-(2-methyl-1-oxopropyl)-2-(phenylmethyl)-L-arabinonoyl]-L-serine, (1-4′)-lactone, N-[2-(1S,2R)-[1,1′-bicyclopropyl]-2-ylphenyl]-3-(difluoromethyl)-1-methyl-1H-pyrazole-4-carboxamide, 2-[(3-bromo-6-quinolinyl)oxy]-N-(1,1-dimethyl-2-butyn-1-yl)-2-(methylthio)acetamide, 2-[(3-bromo-6-quinolinyl)oxy]-N-(1,1-dimethylethyl)butanamide, 2-[(3-bromo-8-methyl-6-quinolinyl)oxy]-N-(1,1-dimethyl-2-propyn-1-yl)-2-(methylthio)acetamide, 2-butoxy-6-iodo-3-propyl-4H-1-benzopyran-4-one, 3-butyn-1-yl N-[6-[[[[(1-methyl-1H-tetrazol-5-yl)-phenylmethylene]amino]oxy]methyl]-2-pyridinyl]carbamate, α-(1-chlorocyclopropyl)-α-[2-(2,2-dichlorocyclopropyl)ethyl]-1H-1,2,4-triazole-1-ethanol, 2-[2-(1-chlorocyclopropyl)-4-(2,2-dichlorocyclopropyl)-2-hydroxybutyl]-1,2-dihydro-3H-1,2,4-triazole-3-thione, (αS)-[3-(4-chloro-2-fluorophenyl)-5-(2,4-difluorophenyl)-4-isoxazolyl]-3-pyridinemethanol, rel-1-[[(2R,3S)-3-(2-chlorophenyl)-2-(2,4-difluorophenyl)-2-oxiranyl]methyl]-1H-1,2,4-triazole, rel-2-[[(2R,3S)-3-(2-chlorophenyl)-2-(2,4-difluorophenyl)-2-oxiranyl]methyl]-1,2-dihydro-3H-1,2,4-triazole-3-thione, rel-1-[[(2R,3S)-3-(2-chlorophenyl)-2-(2,4-difluorophenyl)-2-oxiranyl]methyl]-5-(2-propen-1-ylthio)-1H-1,2,4-triazole, 3-[5-(4-chlorophenyl)-2,3-dimethyl-3-isoxazolidinyl]pyridine, (2-chloro-6-fluorophenyl)methyl 2-[1-[2-[3,5-bis(difluoromethyl)-1H-pyrazol-1-yl]acetyl]-4-piperidinyl]-4-thiazolecarboxylate, N-[4-[[3-[(4-chlorophenyl)methyl]-1,2,4-thiadiazol-5-yl]oxy]-2,5-dimethylphenyl]-N-ethyl-N-methylmethanimidamide, N-[2-[4-[[3-(4-chlorophenyl)-2-propyn-1-yl]oxy]-3-methoxyphenyl]-ethyl]-3-methyl-2-[(methyl sulfonyl)amino]butanamide, N-[2-[4-[[3-(4-chlorophenyl)-2-propyn-1-yl]oxy]-3-methoxyphenyl]ethyl]-3-methyl-2-[(ethylsulfonyl)amino]butanamide, N-[4-[4-chloro-3-(trifluoromethyl)phenoxy]-2,5-dimethylphenyl]-N-ethyl-N-methylmethanimidamide, N-cyclopropyl-3-(difluoromethyl)-5-fluoro-1-methyl-N-[[2-(1-methyl-ethyl)phenyl]methyl]-1H-pyrazole-4-carboxamide, N-[[(cyclopropylmethoxy)amino][6-(difluoromethoxy)-2,3-difluorophenyl]methylene]benzeneacetamide, N-[2-(2,4-dichlorophenyl)-2-methoxy-1-methylethyl]-3-(difluoromethyl)-1-methyl-1H-pyrazole-4-carboxamide, N-(3′,4′-difluoro[1,1′-biphenyl]-2-yl)-3-(trifluoromethyl)-2-pyrazinecarboxamide, 3-(difluoromethyl)-N(2,3-dihydro-1,1,3-trimethyl-1H-inden-4-yl)-1-methyl-1H-pyrazole-4-carboxamide, 3-(difluoromethyl)-N-[4-fluoro-2-(1,1,2,3,3,3-hexa-fluoropropoxy)phenyl]-1-methyl-1H-pyrazole-4-carboxamide, 5,8-difluoro-N-[2-[3-methoxy-4-[[4-(trifluoromethyl)-2-pyridinyl]oxy]phenyl]ethyl]-4-quinazolinamine, 3-(difluoromethyl)-1-methyl-N-[2-(1,1,2,2-tetrafluoroethoxy)phenyl]-1H-pyrazole-4-carboxamide, 1-[4-[4-[5R-[(2,6-difluorophenoxy)methyl]-4,5-dihydro-3-isoxazolyl]-2-thiazolyl]-1-piperdinyl]-2-[5-methyl-3-(trifluoromethyl)-1H-pyrazol-1-yl]ethanone, N-(1,1-dimethyl-2-butyn-1-yl)-2-[(3-ethynyl-6-quinolinyl)oxy]-2-(methylthio)acetamide, 2,6-dimethyl-1H,5H-[1,4]dithiino[2,3-c:5,6-c′]dipyrrole-1,3,5,7(2H,6H)-tetrone, 2-[(3-ethynyl-6-quinolinyl)oxy]-N-[1-(hydroxymethyl)-1-methyl-2-propyn-1-yl]-2-(methylthio)acetamide, 4-fluorophenyl N-[1-[[[1-(4-cyanophenyl)ethyl]sulfonyl]methyl]propyl]carbamate, 5-fluoro-2-[(4-fluorophenyl)methoxy]-4-pyrimidinamine, 5-fluoro-2-[(4-methylphenyl)methoxy]-4-pyrimidinamine, (3S,6S,7R,8R)-3-[[[4-methoxy-3-[[(2-methylpropoxy)carbonyl]oxy]-2-pyridinyl]carbonyl]amino]-6-methyl-4,9-dioxo-8-(phenylmethyl)-1,5-dioxonan-7-yl 2-methylpropanoate, α-(methoxyimino)-N-methyl-2-[[[1-[3-(trifluoro-methyl)phenyl]ethoxy]imino]methyl]benzeneacetamide, [[4-methoxy-2-[[[(3S,7R,8R,9S)-9-methyl-8-(2-methyl-1-oxopropoxy)-2,6-dioxo-7-(phenylmethyl)-1,5-dioxonan-3-yl]-amino]carbonyl]-3-pyridinyl]oxy]methyl 2-methylpropanoate, pentyl N-[6-[[[[(1-methyl-1H-tetrazol-5-yl)phenylmethylene]amino]oxy]methyl]-2-pyridinyl]carbamate, pentyl N-[4-[[[[(1-methyl-1H-tetrazol-5-yl)phenylmethylene]amino]oxy]methyl]-2-thiazolyl]carbamate, and pentyl N-[6-[[[[(Z)-(1-methyl-1H-tetrazol-5-yl)phenylmethylene]amino]oxy]methyl]-2-pyridinyl]carbamate and (1R)-1,2,3,4-tetrahydro-1-naphthalenyl 2-[1-[2-[3,5-bis(difluoromethyl)-1H-pyrazol-1-yl]acetyl]-4-piperidinyl]-4-thiazolecarboxylate. Therefore of note is a fungicidal composition comprising as component (a) a compound of Formula 1 (or an N-oxide or salt thereof) and as component (b) at least one fungicide selected from the preceding list.

Of particular note are combinations of compounds of Formula 1 (or an N-oxide or salt thereof) (i.e. Component (a) in compositions) with azoxystrobin, benzovindiflupyr, bixafen, captan, carpropamid, chlorothalonil, copper hydroxide, copper oxychloride, copper sulfate, cymoxanil, cyproconazole, cyprodinil, diethofencarb, difenoconazole, dimethomorph, epoxiconazole, ethaboxam, fenarimol, fenhexamid, fluazinam, fludioxonil, fluopyram, flusilazole, flutianil, flutriafol, fluxapyroxad, folpet, iprodione, isofetamid, isopyrazam, kresoxim-methyl, mancozeb, mandestrobin, meptyldinocap, metalaxyl (including metalaxyl-Mlmefenoxam), metconazole, metrafenone, myclobutanil, oxathiapiprolin, penflufen, penthiopyrad, phosphorous acid (including salts thereof, e.g., fosetyl-aluminum), picoxystrobin, propiconazole, proquinazid, prothioconazole, pyraclostrobin, pyrimethanil, sedaxane spiroxamine, sulfur, tebuconazole, thiophanate-methyl, trifloxystrobin, zoxamide, α-(1-chlorocyclopropyl)-α-[2-(2,2-dichlorocyclopropyl)ethyl]-1H-1,2,4-triazole-1-ethanol, 2-[2-(1 chlorocyclopropyl)-4-(2,2-dichlorocyclopropyl)-2-hydroxybutyl]-1,2-dihydro-3H-1,2,4-triazole-3-thione, N-[2-(2,4-dichlorophenyl)-2-methoxy-1-methylethyl]-3-(difluoromethyl)-1-methyl-1H-pyrazole-4-carboxamide, 3-(difluoromethyl)-N(2,3-dihydro-1,1,3-trimethyl-1H-inden-4-yl)-1-methyl-1H-pyrazole-4-carboxamide, 1-[4-[4-[5R-(2,6-difluorophenyl)-4, 5-dihydro-3-isoxazolyl]-2-thiazolyl]-1-piperidinyl]-2-[5-methyl-3-(trifluoromethyl)-1H-pyrazol-1-yl]ethanone, 1,1-dimethylethyl N-[6-[[[[(1-methyl-1H-tetrazol-5-yl)phenylmethylene]amino]oxy]methyl]-2-pyridinyl]carbamate, 2,6-dimethyl-1H,5H-[1,4]dithiino[2,3-c:5,6-c′]dipyrrole-1,3,5,7(2H,6H)-tetrone, 5-fluoro-2-[(4-fluoro-phenyl)methoxy]-4-pyrimidinamine, 5-fluoro-2-[(4-methylphenyl)methoxy]-4-pyrimidinamine, (αS)-[3-(4-chloro-2-fluorophenyl)-5-(2,4-difluorophenyl)-4-isoxazolyl]-3-pyridinemethanol, rel-1-[[(2R,3S)-3-(2-chlorophenyl)-2-(2,4-difluorophenyl)-2-oxiranyl]-methyl]-1H-1,2,4-triazole, rel-2-[[(2R,3S)-3-(2-chlorophenyl)-2-(2,4-difluorophenyl)-2-oxiranyl]methyl]-1,2-dihydro-3H-1,2,4-triazole-3-thione, and rel-1-[[(2R,3S)-3-(2-chlorophenyl)-2-(2,4-difluorophenyl)-2-oxiranyl]methyl]-5-(2-propen-1-ylthio)-1H-1,2,4-triazole (i.e. as Component (b) in compositions).

Examples of other biologically active compounds or agents with which compounds of this invention can be formulated are: invertebrate pest control compounds or agents such as abamectin, acephate, acetamiprid, acrinathrin, afidopyropen ([(3S,4R,4aR,6S,6aS,12R,12aS,12bS)-3-[(cyclopropylcarbonyl)oxy]-1,3,4,4a,5,6,6a,12,12a,12b-decahydro-6,12-dihydroxy-4,6a,12b-trimethyl-11-oxo-9-(3-pyridinyl)-2H, 11H-naphtho[2,1-b]pyrano[3,4-e]pyran-4-yl]methyl cyclopropanecarboxylate), amidoflumet (S-1955), avermectin, azadirachtin, azinphos-methyl, bifenthrin, bifenazate, buprofezin, carbofuran, cartap, chlorantraniliprole, chlorfenapyr, chlorfluazuron, chlorpyrifos, chlorpyrifos-methyl, chromafenozide, clothianidin, cyantraniliprole (3-bromo-1-(3-chloro-2-pyridinyl)-N-[4-cyano-2-methyl-6-[(methylamino)carbonyl]phenyl]-1H-pyrazole-5-carboxamide), cyclaniliprole (3-bromo-N-[2-bromo-4-chloro-6-[[(1-cyclopropylethyl)amino]carbonyl]phenyl]-1-3-chloro-2-pyridinyl)-1H-pyrazole-5-carboxamide), cycloxaprid ((5S,8R)-1-[(6-chloro-3-pyridinyl)methyl]-2,3,5,6,7,8-hexahydro-9-nitro-5,8-epoxy-1H-imidazo[1,2-a]azepine), cyflumetofen, cyfluthrin, beta-cyfluthrin, cyhalothrin, lambda-cyhalothrin, cypermethrin, cyromazine, deltamethrin, diafenthiuron, diazinon, dieldrin, diflubenzuron, dimefluthrin, dimethoate, dinotefuran, diofenolan, emamectin, endosulfan, esfenvalerate, ethiprole, fenothiocarb, fenoxycarb, fenpropathrin, fenvalerate, fipronil, flonicamid, flubendiamide, flucythrinate, flufenoxystrobin (methyl (αE)-2-[[2-chloro-4-(trifluoromethyl)phenoxy]methyl]-α-(methoxymethylene)benzeneacetate), flufensulfone (5-chloro-2-[(3,4,4-trifluoro-3-buten-1-yl)sulfonyl]thiazole), flupiprole (1-[2,6-dichloro-4-(trifluoromethyl)phenyl]-5-[(2-methyl-2-propen-1-yl)amino]-4-[(trifluoromethyl)sulfinyl]-1H-pyrazole-3-carbonitrile), flupyradifurone (4-[[(6-chloro-3-pyridinyl)methyl](2,2-difluoroethyl)amino]-2(5H)-furanone), tau-fluvalinate, flufenerim (UR-50701), flufenoxuron, fonophos, halofenozide, heptafluthrin ([2,3,5,6-tetrafluoro-4-(methoxymethyl)phenyl]methyl 2,2-dimethyl-3-[(1Z)-3,3,3-trifluoro-1-propen-1-yl]cyclopropanecarboxylate), hexaflumuron, hydramethylnon, imidacloprid, indoxacarb, isofenphos, lufenuron, malathion, meperfluthrin ([2,3,5,6-tetrafluoro-4-(methoxymethyl)phenyl]methyl (1R,3S)-3-(2,2-dichloroethenyl)-2,2-dimethylcyclopropanecarboxylate), metaflumizone, metaldehyde, methamidophos, methidathion, methomyl, methoprene, methoxychlor, methoxyfenozide, metofluthrin, milbemycin oxime, momfluorothrin ([2,3,5,6-tetrafluoro-4-(methoxymethyl)phenyl]methyl 3-(2-cyano-1-propen-1-yl)-2,2-dimethylcyclopropanecarboxylate), monocrotophos, nicotine, nitenpyram, nithiazine, novaluron, noviflumuron (XDE-007), oxamyl, pyflubumide (1,3,5-trimethyl-N-(2-methyl-1-oxopropyl)-N-[3-(2-methylpropyl)-4-[2,2,2-trifluoro-1-methoxy-1-(trifluoromethyl)ethyl]phenyl]-1H-pyrazole-4-carboxamide), parathion, parathion-methyl, permethrin, phorate, phosalone, phosmet, phosphamidon, pirimicarb, profenofos, profluthrin, pymetrozine, pyrafluprole, pyrethrin, pyridalyl, pyrifluquinazon, pyriminostrobin (methyl (αE)-2-[[[2-[(2,4-dichlorophenyl)amino]-6-(trifluoromethyl)-4-pyrimidinyl]oxy]methyl]-α-(methoxymethylene)benzeneacetate), pyriprole, pyriproxyfen, rotenone, ryanodine, spinetoram, spinosad, spirodiclofen, spiromesifen (BSN 2060), spirotetramat, sulfoxaflor, sulprofos, tebufenozide, teflubenzuron, tefluthrin, terbufos, tetrachlorvinphos, tetramethylfluthrin, thiacloprid, thiamethoxam, thiodicarb, thiosultap-sodium, tolfenpyrad, tralomethrin, triazamate, trichlorfon and triflumuron; and biological agents including entomopathogenic bacteria, such as Bacillus thuringiensis subsp. aizawai, Bacillus thuringiensis subsp. kurstaki, and the encapsulated delta-endotoxins of Bacillus thuringiensis (e.g., Cellcap, MPV, MPVII); entomopathogenic fungi, such as green muscardine fungus; and entomopathogenic virus including baculovirus, nucleopolyhedro virus (NPV) such as HzNPV, AfNPV; and granulosis virus (GV) such as CpGV.

Compounds of this invention and compositions thereof can be applied to plants genetically transformed to express proteins toxic to invertebrate pests (such as Bacillus thuringiensis delta-endotoxins). The effect of the exogenously applied fungicidal compounds of this invention may be synergistic with the expressed toxin proteins.

General references for agricultural protectants (i.e. insecticides, fungicides, nematocides, acaricides, herbicides and biological agents) include The Pesticide Manual, 13th Edition, C. D. S. Tomlin, Ed., British Crop Protection Council, Farnham, Surrey, U. K., 2003 and The BioPesticide Manual, 2nd Edition, L. G. Copping, Ed., British Crop Protection Council, Farnham, Surrey, U. K., 2001.

For embodiments where one or more of these various mixing partners are used, the weight ratio of these various mixing partners (in total) to the compound of Formula 1 is typically between about 1:3000 and about 3000:1. Of note are weight ratios between about 1:300 and about 300:1 (for example ratios between about 1:30 and about 30:1). One skilled in the art can easily determine through simple experimentation the biologically effective amounts of active ingredients necessary for the desired spectrum of biological activity. It will be evident that including these additional components may expand the spectrum of diseases controlled beyond the spectrum controlled by the compound of Formula 1 alone.

In certain instances, combinations of a compound of this invention with other biologically active (particularly fungicidal) compounds or agents (i.e. active ingredients) can result in a greater-than-additive (i.e. synergistic) effect. Reducing the quantity of active ingredients released in the environment while ensuring effective pest control is always desirable. When synergism of fungicidal active ingredients occurs at application rates giving agronomically satisfactory levels of fungal control, such combinations can be advantageous for reducing crop production cost and decreasing environmental load.

Also in certain instances, combinations of a compound of the invention with other biologically active compounds or agents can result in a less-than-additive (i.e. safening) effect on organisms beneficial to the agronomic environment. For example, a compound of the invention may safen a herbicide on crop plants or protect a beneficial insect species (e.g., insect predators, pollinators such as bees) from an insecticide.

Fungicides of note for formulation with compounds of Formula 1 to provide mixtures useful in seed treatment include but are not limited to amisulbrom, azoxystrobin, boscalid, carbendazim, carboxin, cymoxanil, cyproconazole, difenoconazole, dimethomorph, fluazinam, fludioxonil, flufenoxystrobin, fluquinconazole, fluopicolide, fluoxastrobin, flutriafol, fluxapyroxad, ipconazole, iprodione, metalaxyl, mefenoxam, metconazole, myclobutanil, paclobutrazole, penflufen, picoxystrobin, prothioconazole, pyraclostrobin, sedaxane, silthiofam, tebuconazole, thiabendazole, thiophanate-methyl, thiram, trifloxystrobin and triticonazole.

Invertebrate pest control compounds or agents with which compounds of Formula 1 can be formulated to provide mixtures useful in seed treatment include but are not limited to abamectin, acetamiprid, acrinathrin, afidopyropen, amitraz, avermectin, azadirachtin, bensultap, bifenthrin, buprofezin, cadusafos, carbaryl, carbofuran, cartap, chlorantraniliprole, chlorfenapyr, chlorpyrifos, clothianidin, cyantraniliprole, cyclaniliprole, cyfluthrin, beta-cyfluthrin, cyhalothrin, gamma-cyhalothrin, lambda-cyhalothrin, cypermethrin, alpha-cypermethrin, zeta-cypermethrin, cyromazine, deltamethrin, dieldrin, dinotefuran, diofenolan, emamectin, endosulfan, esfenvalerate, ethiprole, etofenprox, etoxazole, fenothiocarb, fenoxycarb, fenvalerate, fipronil, flonicamid, flubendiamide, fluensulfone, flufenoxuron, flufiprole, flupyradifurone, fluvalinate, formetanate, fosthiazate, heptafluthrin, hexaflumuron, hydramethylnon, imidacloprid, indoxacarb, lufenuron, meperfluthrin, metaflumizone, methiocarb, methomyl, methoprene, methoxyfenozide, momfluorothrin, nitenpyram, nithiazine, novaluron, oxamyl, pyflubumide, pymetrozine, pyrethrin, pyridaben, pyriminostrobin, pyridalyl, pyriproxyfen, ryanodine, spinetoram, spinosad, spirodiclofen, spiromesifen, spirotetramat, sulfoxaflor, tebufenozide, tetramethrin, tetramethylfluthrin, thiacloprid, thiamethoxam, thiodicarb, thiosultap-sodium, tralomethrin, triazamate, triflumuron, Bacillus thuringiensis delta-endotoxins, strains of Bacillus thuringiensis and strains of Nucleo polyhydrosis viruses.

Compositions comprising compounds of Formula 1 useful for seed treatment can further comprise bacteria and fungi that have the ability to provide protection from the harmful effects of plant pathogenic fungi or bacteria and/or soil born animals such as nematodes. Bacteria exhibiting nematicidal properties may include but are not limited to Bacillus firmus, Bacillus cereus, Bacillius subtiliis and Pasteuria penetrans. A suitable Bacillus firmus strain is strain CNCM 1-1582 (GB-126) which is commercially available as BioNem™. A suitable Bacillus cereus strain is strain NCMM 1-1592. Both Bacillus strains are disclosed in U.S. Pat. No. 6,406,690. Other suitable bacteria exhibiting nematicidal activity are B. amyloliquefaciens IN937a and B. subtilis strain GB03. Bacteria exhibiting fungicidal properties may include but are not limited to B. pumilus strain GB34. Fungal species exhibiting nematicidal properties may include but are not limited to Myrothecium verrucaria, Paecilomyces lilacinus and Purpureocillium lilacinum.

Seed treatments can also include one or more nematicidal agents of natural origin such as the elicitor protein called harpin which is isolated from certain bacterial plant pathogens such as Erwinia amylovora. An example is the Harpin-N-Tek seed treatment technology available as N-Hibit™ Gold CST.

Seed treatments can also include one or more species of legume-root nodulating bacteria such as the microsymbiotic nitrogen-fixing bacteria Bradyrhizobium japonicum. These inocculants can optionally include one or more lipo-chitooligosaccharides (LCOs), which are nodulation (Nod) factors produced by rhizobia bacteria during the initiation of nodule formation on the roots of legumes. For example, the Optimize® brand seed treatment technology incorporates LCO Promoter Technology™ in combination with an inocculant.

Seed treatments can also include one or more isoflavones which can increase the level of root colonization by mycorrhizal fungi. Mycorrhizal fungi improve plant growth by enhancing the root uptake of nutrients such as water, sulfates, nitrates, phosphates and metals. Examples of isoflavones include, but are not limited to, genistein, biochanin A, formononetin, daidzein, glycitein, hesperetin, naringenin and pratensein. Formononetin is available as an active ingredient in mycorrhizal inocculant products such as PHC Colonize® AG.

Seed treatments can also include one or more plant activators that induce systemic acquired resistance in plants following contact by a pathogen. An example of a plant activator which induces such protective mechanisms is acibenzolar-S-methyl.

The control efficacy of compounds of this invention on specific pathogens is demonstrated in TABLE A below. The pathogen control protection afforded by the compounds of this invention is not limited, however, to the species described in Tests A-E below. Descriptions of the compounds are provided in Index Table A below. The following abbreviations are used in the Index Table which follow: n means normal, Me means methyl, Et means ethyl, Pr means propyl, Ph means phenyl, “Cmpd. No.” means compound number, and “Ex.” stands for “Example” and is followed by a number indicating in which example the compound is prepared. In Index Table A the numerical value reported in the column “AP⁺ (M+1)”, is the molecular weight of the observed molecular ion formed by addition of H+(molecular weight of 1) to the molecule having the greatest isotopic abundance (i.e. M). The presence of molecular ions containing one or higher atomic weight isotopes of lower abundance (e.g., ³⁷Cl, ⁸¹Br) is not reported. The reported M+1 peaks were observed by mass spectrometry using atmospheric pressure chemical ionization (AP⁺).

INDEX TABLE A

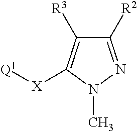

| | | | | | | AP⁺ |
|---|---|---|---|---|---|---|
| Cmpd. No. | R² | Q¹ | X | R³ | m.p. (° C.) | (M + 1) |
| 1 | Me | 4-F—Ph | NH | n-octyl | | 318 |
| 2 | Me | 2-Cl-4-F—Ph | NH | n-octyl | | 352 |
| 3 | Me | 2-Cl-4-F—Ph | NH | 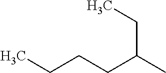 | | 338 |
| 4 | Me | 2,6-di-F-4-NO₂—Ph | NH | 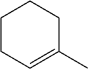 | 147-150 | |
| 5 | Me | 2,6-di-F-4-NH₂—Ph | NH | 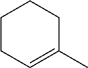 | 178-181 | |
| 6 (Ex. 1) | Me | 2,6-di-F-4-Cl—Ph | NH | 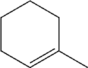 | 142-145 | |
| 7 | Me | 2,6-di-F—Ph | O | CH₃CH₂C(═O) | 210-212 | |
| 8 | Br | 2-Cl-4-F—Ph | CHOH | 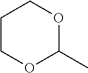 | 138-140 | |
| 9 | Br | 2-Br-4-F—Ph | CHOH | 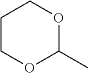 | 119-121 | |
| 10 | Br | 2,4-di-F—Ph | CHOH | 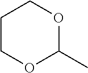 | 134-136 | |
| 11 | Me | 2-Cl-4-F—Ph | CHOH | cyclohexyl | 171-173 | |
| 12 (Ex. 5) | Me | 2,4-di-F—Ph | CHOH | cyclohexyl | 154-156 | |
| 13 | Me | 2-Cl-4-F—Ph | NH | 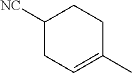 | 110-112 | |
| 14 | Me | 4-F—Ph | NH | 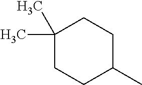 | 187-189 | |
| 15 | Me | 2-Cl-4-F—Ph | NH | 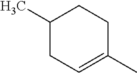 | 68-70 | |
| 16 | Me | 2,4-di-F—Ph | NH | 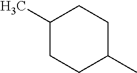 | | 320 |
| 17 | Me | 2,4-di-F—Ph | NH | 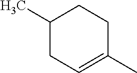 | | 318 |
| 18 | Me | 2,4-di-F—Ph | NH | 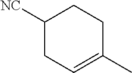 | 120-122 | |
| 19 | Me | 2,4-di-F—Ph | NH | 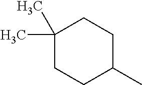 | | 334 |
| 20 | Me | 2,4-di-F—Ph | NH | 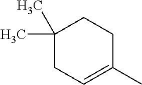 | 100-103 | |
| 21 | Me | 2-Cl-4-F—Ph | NH | 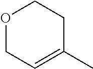 | 89-92 | |
| 22 | Me | 2,6-di-F-4-Cl—Ph | NH | 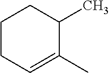 | | 353 |
| 23 (Ex. 4) | Me | 2,6-di-F-4-Cl—Ph | NH | 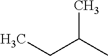 | 80-84 | |
| 24 (Ex. 3) | Me | 2,6-di-F-4-Cl—Ph | NH | 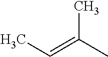 | 140-142 | |
| 25 | Me | 2,6-di-F—Ph | O | 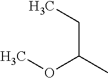 | | 297 |
| 26 | Me | 2-Cl-4-F—Ph | NH | 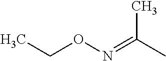 | 117-120 | |
| 27 | Me | 2-Cl-4-F—Ph | NH | 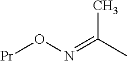 | | 339 |
| 28 | Me | 2-Cl-4-F—Ph | NH | 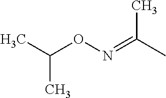 | | 339 |
| 29 | Me | 2-Cl-4-F—Ph | N—Et | 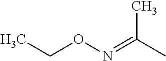 | | 353 |
| 30 | Me | 2-Cl-4-F—Ph | NH | 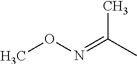 | 133-136 | |
| 31 | Me | 2-Cl-4-F—Ph | NH | 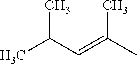 | | 322 |
| 32 | Me | 2-Cl-4-F—Ph | NH | CH₃C(═O) | 89-92 | |
| 33 | Me | 2-Cl-4-F—Ph | N—Me | 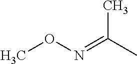 | | 325 |
| 34 | Me | 2-Cl-4-F—Ph | NH | 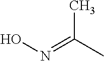 | 156-159 | |
| 35 | Me | 2-Cl-4-F—Ph | O | CH₂═CH— | | 267 |
| 36 | Me | 2,6-di-F-4-NO₂—Ph | NH | CH₃C(═CH₂)— | 102-105 | |
| 37 | Me | 2-Cl-4-F—Ph | O | 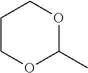 | 70-74 | |
| 38 | Me | 2-Cl-4-F—Ph | O | 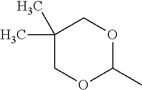 | | 355 |
| 39 | Me | 2-Cl-4-F—Ph | O | CH₃C(═CH₂)— | | 281 |
| 40 | Me | 2-Cl-4-F—Ph | O | 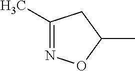 | | 324 |
| 41 | Me | 2-Cl-4-F—Ph | O | CH₃CH(OH)— | | 285 |
| 42 | Me | 2-Cl-4-F—Ph | O | OH-N═CH— | 181-184 | |
| 43 | Me | 2,6-di-F-4-Cl—Ph | NH | 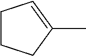 | 113-116 | |
| 44 | Me | 2,6-di-F—Ph | O | 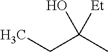 | 82-85 | |
| 45 | Me | 2,6-di-F—Ph | O | 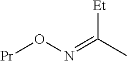 | | 338 |
| 46 (Ex. 2) | Me | 2,6-di-F-4-Cl—Ph | NH | cyclohexyl | 128-132 | |
| 47 | Me | 2,6-di-F—Ph | O | OH—N═C(Et)— | 110-113 | |
| 48 | Me | 2,6-di-F—Ph | O | 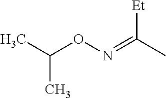 | | 338 |
| 49 | Me | 2,6-di-F—Ph | O | 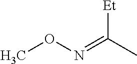 | | 310 |
| 50 | Me | 2,6-di-F—Ph | O | 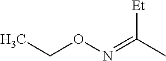 | | 324 |
| 51 | Me | 2,6-di-F—Ph | O | 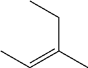 | | 293 |
| 52 | Me | 2,6-di-F-4-Cl—Ph | NH | cyclopentyl | 139-142 | |
| 53 | Me | 2-Cl-4-F—Ph | O | 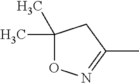 | 118-122 | |
| 54 | Me | 2-Cl-4-F—Ph | O | 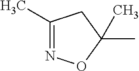 | 100-103 | |
| 55 | Me | 2-Cl-4-F—Ph | O | 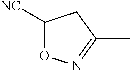 | 147-151 | |
| 56 | Me | 2-Cl-4-F—Ph | NH | 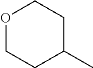 | 143-146 | |
| 57 | Me | 4-F—Ph | NH | 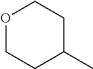 | | 290 |
| 58 | Me | 2,4-di-F—Ph | NH | 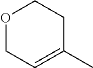 | | 305 |
| 59 | Me | 2,4-di-F—Ph | NH | 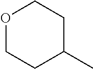 | 135-138 | |
| 60 | Me | 2-Cl-4-F—Ph | NH | 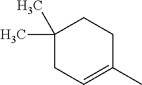 | | 348 |
| 61 | Me | 2-Cl-4-F—Ph | NH | 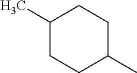 | | 336 |
| 62 | Me | 2-Cl-4-F—Ph | NH | 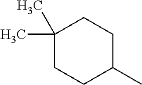 | | 350 |
| 63 | Me | 2,4-di-F—Ph | NH | 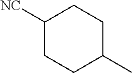 | | 331 |
| 64 | Me | 4-F—Ph | NH | 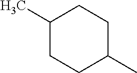 | | 302 |
| 65 | Me | 2-Cl-4-F—Ph | NH | 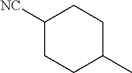 | 153-155 | |

### BIOLOGICAL EXAMPLES OF THE INVENTION

General protocol for preparing test suspensions for Tests A-E: the test compounds were first dissolved in acetone in an amount equal to 3% of the final volume and then suspended at the desired concentration (in ppm) in acetone and purified water (50/50 mix by volume) containing 250 ppm of the surfactant Trem® 014 (polyhydric alcohol esters). The resulting test suspensions were then used in Tests A-E. Spraying a 200 ppm test suspension to the point of run-off on the test plants was the equivalent of a rate of 800 g/ha.

### Test A

The test suspension was sprayed to the point of run-off on wheat seedlings. The following day the seedlings were inoculated with a spore suspension of Puccinia recondita f. sp. tritici (the causal agent of wheat leaf rust) and incubated in a saturated atmosphere at 20° C. for 24 h, and then moved to a growth chamber at 20° C. for 7 days, after which time visual disease ratings were made.

### Test B

The test suspension was sprayed to the point of run-off on wheat seedlings. The following day the seedlings were inoculated with a spore suspension of Septoria tritici (the causal agent of wheat leaf blotch) and incubated in a saturated atmosphere at 24° C. for 48 h, and then moved to a growth chamber at 20° C. for 19 days, after which time visual disease ratings were made.

### Test C

The test suspension was sprayed to the point of run-off on tomato seedlings. The following day the seedlings were inoculated with a spore suspension of Botrytis cinerea (the causal agent of tomato Botrytis) and incubated in a saturated atmosphere at 20° C. for 48 h, and then moved to a growth chamber at 24° C. for 3 days, after which time visual disease ratings were made.

### Test D

The test suspension was sprayed to the point of run-off on wheat seedlings. The following day the seedlings were inoculated with a spore dust of Blumeria graminis f sp. tritici, (also known as Erysiphe graminis f sp. tritici, the causal agent of wheat powdery mildew) and incubated in a growth chamber at 20° C. for 8 days, after which time visual disease ratings were made.

### Test E

The test suspension was sprayed to the point of run-off on wheat seedlings. The following day the seedlings were inoculated with a spore suspension of Septoria nodorum (the causal agent of Septoria glume blotch) and incubated in a saturated atmosphere at 20° C. for 48 h, and then moved to a growth chamber at 20° C. for 9 days, after which time visual disease ratings were made.

Results for Tests A-E are given in Table A. In the Table, a rating of 100 indicates 100% disease control and a rating of 0 indicates no disease control (relative to the controls). A dash (-) indicates no test results. All results are for 200 ppm except where followed by an asterisk “*” which indicates a 250 ppm test suspension was used or a double asterisk “**” which indicates a 50 ppm test suspension was used.

TABLE A

| Cmpd. No | Test A | Test B | Test C | Test D | Test E |
|---|---|---|---|---|---|
| 1 | 41 | 0 | 0 | 63 | 0 |
| 2 | 0 | 0 | 0 | 0 | 0 |
| 3 | 9 | 0 | — | 0 | — |
| 4 | 32 | 93 | 92 | 0 | 0 |
| 5 | 0 | 1 | 0 | 0 | 0 |
| 6 | 19 | 84 | 98 | 0 | 0 |
| 7 | 0 | 62 | 0 | 0 | 0 |
| 8 | 0* | 5* | 0* | 72* | — |
| 9 | 0* | 0* | 0* | 64* | — |
| 10 | 0* | 0* | 0* | 90* | — |
| 11 | 86* | 100* | 24* | 93* | — |
| 12 | 94* | 100* | 61* | 97* | — |
| 13 | 67* | 97* | 79* | 13* | 0* |
| 14 | 67* | 33* | 0* | 27* | 62* |
| 15 | 53* | 32* | 6* | 0* | 0* |
| 16 | 53* | 0* | 19* | 97* | 19* |
| 17 | 53* | 3* | 39* | 27* | 0* |
| 18 | 19* | 79* | 73* | 0* | — |
| 19 | 0* | 1* | 0* | 96* | — |
| 20 | 0* | 0* | 0* | 48* | — |
| 21 | 0* | 17* | 42* | 13* | — |
| 22 | 68 | — | 99 | 56 | — |
| 23 | 68 | — | 98 | 95 | — |
| 24 | 94 | 96 | 85 | 100 | — |
| 25 | 99* | 6* | 0* | 0* | — |
| 26 | 0* | 33* | 0* | 0* | — |
| 27 | 9* | 17* | 0* | 69* | — |
| 28 | 32* | 24* | 0* | 0* | — |
| 29 | 55* | 14* | 0* | 0* | — |
| 30 | 80* | 14* | 0* | 0* | — |
| 31 | 55* | 6* | 0* | 48* | — |
| 32 | 19* | — | 56* | 0* | — |
| 33 | 17** | 0** | 7** | 0** | — |
| 34 | 28* | 76* | 0* | 64* | 0* |
| 35 | 8* | 0* | 0* | 26* | — |
| 36 | 55* | 88* | 0* | 27* | 0* |
| 37 | 52* | 5* | 0* | 63* | — |
| 38 | 52* | 93* | 0* | 94* | — |
| 39 | 15* | 0* | 0* | 13* | — |
| 40 | 30* | 36* | 33* | 81* | — |
| 41 | 0* | 0* | 0* | 46* | — |
| 42 | 9* | 0* | 7* | 81* | — |
| 43 | 68 | 92 | 0 | 0 | 0 |
| 44 | 0 | 15 | 3 | 43 | 0 |
| 45 | 0 | 3 | 0 | 0 | 0 |
| 46 | 92 | 99 | 95 | 98 | 0 |
| 47 | 0 | 81 | 0 | 89* | 0 |
| 48 | 0 | 32 | 18 | 0 | 0 |
| 49 | 0 | 16 | 39 | 0 | 0 |
| 50 | 0 | 40 | 3 | 0 | 0 |
| 51 | 0 | 0 | 15 | 0 | 0 |
| 52 | 89 | 99 | 96 | 98 | 0 |
| 53 | 0* | 2* | 0* | 90* | — |
| 54 | 0* | 0* | 0* | 92* | — |
| 55 | 0* | 0* | 0* | 73* | — |
| 56 | 55* | 100* | 100* | 99* | — |
| 57 | 0* | 44* | 0* | 89* | — |
| 58 | 0* | 25* | 0* | 0* | — |
| 59 | 41* | 92* | 24* | 98* | — |
| 60 | 19* | 16* | 0* | 0* | — |
| 61 | 0* | 53* | 0* | 97* | — |
| 62 | 68* | 9* | 0* | 96* | — |
| 63 | 86* | 55* | 0* | 96* | — |
| 64 | 55* | 58* | 0* | 96* | — |
| 65 | 55* | 54* | 0* | 13* | — |

What is claimed is:

1. A compound selected from Formula 1, N-oxides and salts thereof, 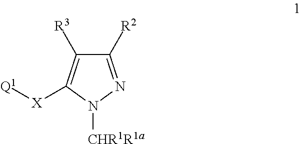
wherein
Q¹ is a phenyl ring or a naphthalenyl ring system, each ring or ring system optionally substituted with up to 5 substituents independently selected from R⁴; or a 5- to 6-membered fully unsaturated heterocyclic ring or an 8- to 10-membered heteroaromatic bicyclic ring system, each ring or ring system containing ring members selected from carbon atoms and 1 to 4 heteroatoms independently selected from up to 2 O, up to 2 S and up to 4 N atoms, wherein up to 3 carbon ring members are independently selected from C(═O) and C(═S), and the sulfur atom ring members are independently selected from S(═O)ᵤ(═NR¹¹)ᵥ, each ring or ring system optionally substituted with up to 5 substituents independently selected from R⁴ on carbon atom ring members and selected from cyano, C₁-C₄ alkyl, C₂-C₄ alkenyl, C₂-C₄ alkynyl, C₃-C₆ cycloalkyl, C₂-C₄ alkoxyalkyl, C₁-C₄ alkoxy, C₂-C₄ alkylcarbonyl, C₂-C₄ alkoxycarbonyl, C₂-C₄ alkylaminoalkyl and C₃-C₄ dialkylaminoalkyl on nitrogen atom ring members;
X is O, S(═O)ₘ, NR⁵ or CR^{6a}OR^{6b};
R¹ is H, cyano, halogen, C₁-C₃ alkyl, C₁-C₃ haloalkyl, C₂-C₃ alkenyl, C₂-C₃ alkynyl, cyclopropyl, C₂-C₃ alkoxyalkyl, C₁-C₃ alkoxy or C₁-C₃ haloalkoxy;
R^{1a} is H; or
R^{1a} and R¹ are taken together with the carbon atom to which they are attached to form a cyclopropyl ring optionally substituted with up to 2 substituents independently selected from halogen and methyl;
R² is H, cyano, halogen, C₁-C₃ alkyl, C₁-C₃ haloalkyl, C₂-C₃ alkenyl, C₂-C₃ haloalkenyl, C₂-C₃ alkynyl, C₂-C₃ cyanoalkyl, C₁-C₃ hydroxyalkyl, C₁-C₃ alkoxy or C₁-C₃ alkylthio; or cyclopropyl optionally substituted with up to 2 substituents independently selected from halogen and methyl;
R³ is C₁-C₈ alkyl, C₁-C₈ haloalkyl, C₂-C₈ alkenyl, C₂-C₈ haloalkenyl, C₂-C₈ alkynyl, C₂-C₈ haloalkynyl, C₂-C₈ cyanoalkyl, C₁-C₈ hydroxyalkyl, C₁-C₈ nitroalkyl, C₂-C₈ alkoxyalkyl, C₂-C₈ haloalkoxyalkyl, C₄-C₁₀ cycloalkoxyalkyl, C₃-C₈ alkoxyalkoxyalkyl, C₂-C₈ alkylthioalkyl, C₂-C₈ haloalkylthioalkyl, C₂-C₈ alkylsulfinylalkyl, C₂-C₈ haloalkylsulfinylalkyl, C₂-C₈ alkylsulfonylalkyl, C₂-C₈ haloalkylsulfonylalkyl, C₃-C₈ alkylcarbonyl, C₃-C₈ alkylcarbonylalkyl, C₃-C₈ haloalkylcarbonylalkyl, C₃-C₈ alkoxycarbonylalkyl, C₃-C₈ haloalkoxycarbonylalkyl, C₂-C₈ alkylaminoalkyl, C₂-C₈ haloalkylaminoalkyl, C₃-C₈ dialkylaminoalkyl, C₃-C₈ alkylaminocarbonylalkyl, C₄-C₁₀ dialkylaminocarbonylalkyl, C₄-C₁₀ cycloalkylaminoalkyl, —CR^{7a}═NOR^{7b} or —(CH₂)ₙW; or C₃-C₈ cycloalkyl, C₃-C₈ cycloalkenyl or C₄-C₁₀ cycloalkylalkyl, each optionally substituted with up to 3 substituents independently selected from R⁸;
W is a 3- to 7-membered saturated or partially unsaturated heterocyclic ring containing ring members selected from carbon atoms and 1 to 4 heteroatoms independently selected from up to 2 O, up to 2 S and up to 3 N atoms, wherein up to 3 carbon atom ring members are independently selected from C(═O) and C(═S), the ring optionally substituted with up to 3 substituents independently selected from R⁹ on carbon atom ring members and R¹⁰ on nitrogen atom ring members;
each R⁴ is independently amino, cyano, halogen, hydroxy, nitro, C₁-C₈ alkyl, C₁-C₈ haloalkyl, C₂-C₈ alkenyl, C₂-C₈ haloalkenyl, C₂-C₈ alkynyl, C₂-C₈ haloalkynyl, C₁-C₈ nitroalkyl, C₂-C₈ nitroalkenyl, C₃-C₈ cycloalkyl, C₃-C₈ halocycloalkyl, C₁-C₈ alkylthio, C₁-C₈ haloalkylthio, C₁-C₈ alkylsulfinyl, C₁-C₈ haloalkylsulfinyl, C₁-C₈ alkylsulfonyl, C₁-C₈ haloalkylsulfonyl, C₁-C₈ alkoxy, C₁-C₈ haloalkoxy, C₂-C₈ alkenyloxy, C₂-C₈ haloalkenyloxy, C₃-C₈ alkynyloxy, C₃-C₈ haloalkynyloxy, C₄-C₁₂ cycloalkylalkoxy, C₂-C₈ alkylcarbonyloxy, C₂-C₈ alkylaminoalkoxy, C₃-C₈ dialkylaminoalkoxy, C₂-C₈ alkylcarbonyl, C₁-C₈ alkylamino, C₂-C₈ dialkylamino, C₂-C₈ alkylcarbonylamino, —CH(═O), —NHCH(═O), —SF₅ or —SC≡N;
R⁵ is H, C₁-C₃ alkyl, C₂-C₆ cyanoalkyl or C₂-C₆ alkoxyalkyl;
R^{6a} is H or C₁-C₆ alkyl;
R^{6b} is H, —CH(═O), C₂-C₆ alkoxyalkyl, C₂-C₆ alkylcarbonyl or C₂-C₆ alkoxycarbonyl;
R^{7a} is H, C₁-C₃ alkyl or C₁-C₃ haloalkyl;
R^{7b} is H, C₁-C₃ alkyl, C₂-C₄ alkenyl, C₂-C₄ alkynyl, C₁-C₃ haloalkyl, C₂-C₄ haloalkenyl, C₃-C₄ cycloalkyl, C₄-C₈ cycloalkylalkyl or C₃-C₄ halocycloalkyl;
each R⁸ is independently cyano, halogen, hydroxy, C₁-C₃ alkyl, C₁-C₃ haloalkyl, C₃-C₆ cycloalkyl, C₁-C₃ alkoxy, C₁-C₃ haloalkoxy or C₂-C₄ alkoxyalkyl;
each R⁹ is independently cyano, halogen, C₁-C₃ alkyl, C₁-C₃ haloalkyl, C₁-C₃ alkoxy, C₁-C₃ haloalkoxy or C₂-C₄ alkoxyalkyl;
each R¹⁰ is independently cyano, C₁-C₃ alkyl or C₁-C₃ alkoxy;
each R¹¹ is independently H, cyano, C₁-C₃ alkyl or C₁-C₃ haloalkyl;
each u and v are independently 0, 1 or 2 in each instance of S(═O)ᵤ(═NR¹¹)ᵥ, provided that the sum of u and v is 0, 1 or 2;
m is 0, 1 or 2; and
n is 0 or 1.

2. A compound of claim 1 wherein:
Q¹ is a phenyl ring substituted with 1 to 3 substituents independently selected from R⁴; or a pyridinyl, pyrimidinyl, pyrazinyl or pyridazinyl ring, each ring optionally substituted with up to 3 substituents independently selected from R⁴;
X is O, NR⁵ or CR^{6a}OR^{6b};
R¹ is H or C₁-C₃ alkyl;
R^{1a} is H;
R² is Br, Cl or methyl;
R³ is C₁-C₆ alkyl, C₁-C₆ haloalkyl, C₂-C₆ alkenyl, C₂-C₆ haloalkenyl, C₂-C₆ alkynyl, C₂-C₆ cyanoalkyl, C₂-C₆ alkoxyalkyl, C₂-C₆ haloalkoxyalkyl, C₄-C₁₀ cycloalkoxyalkyl, C₃-C₆ alkoxyalkoxyalkyl, C₂-C₆ alkylthioalkyl, C₂-C₆ alkylsulfinylalkyl, C₂-C₆ haloalkylsulfinylalkyl, C₂-C₆ alkylsulfonylalkyl, C₂-C₆ haloalkylsulfonylalkyl, C₃-C₆ alkylcarbonylalkyl, C₃-C₆ haloalkylcarbonylalkyl, C₃-C₆ alkoxycarbonylalkyl, C₂-C₆ alkylaminoalkyl, C₃-C₆ dialkylaminoalkyl, C₃-C₆ alkylaminocarbonylalkyl or —(CH₂)ₙW; or C₃-C₆ cycloalkyl, C₃-C₆ cycloalkenyl or C₄-C₇ cycloalkylalkyl, each optionally substituted with up to 3 substituents independently selected from R⁸;
W is a 5- to 6-membered saturated or partially unsaturated heterocyclic ring containing ring members selected from carbon atoms and 1 to 3 heteroatoms independently selected from up to 2 O, up to 2 S and up to 3 N atoms, wherein up to 2 carbon atom ring members are selected from C(═O), the ring optionally substituted with up to 3 substituents independently selected from R⁹ on carbon atom ring members and R¹⁰ on nitrogen atom ring members;
each R⁴ is independently halogen;
R⁵ is H, methyl, cyanomethyl or C₂-C₃ alkoxyalkyl;
R^{6a} is H or methyl;
R^{6b} is H;
each R⁸ is independently halogen, methyl, halomethyl, cyclopropyl, methoxy or C₂-C₄ alkoxyalkyl;
each R⁹ is independently halogen, methyl, halomethyl, methoxy or C₂-C₄ alkoxyalkyl; and
each R¹⁰ is methyl.

3. A compound of claim 2 wherein
Q¹ is a phenyl ring substituted with 1 to 3 substituents independently selected from R⁴;
X is O, NH or CHOH;
R¹ is H;
R² is methyl;
R³ is C₁-C₆ alkyl, C₁-C₆ haloalkyl, C₂-C₆ alkenyl, C₂-C₆ haloalkenyl, C₂-C₆ alkoxyalkyl, C₃-C₆ alkoxyalkoxyalkyl, C₂-C₆ alkylthioalkyl, C₂-C₆ alkylsulfinylalkyl, C₂-C₆ haloalkylsulfinylalkyl, C₂-C₆ alkylsulfonylalkyl, C₂-C₆ haloalkylsulfonylalkyl, C₃-C₆ alkylcarbonylalkyl, C₃-C₆ haloalkylcarbonylalkyl, C₃-C₆ alkoxycarbonylalkyl or —(CH₂)ₙW; or C₃-C₆ cycloalkyl, C₃-C₆ cycloalkenyl or C₄-C₇ cycloalkylalkyl, each optionally substituted with up to 2 substituents independently selected from R⁸;
W is a 5- to 6-membered saturated or partially unsaturated heterocyclic ring containing ring members selected from carbon atoms and 1 to 3 heteroatoms independently selected from up to 2 O, up to 2 S and up to 3 N atoms, the ring optionally substituted with up to 2 substituents independently selected from R⁹ on carbon atom ring members and R¹⁰ on nitrogen atom ring members;
each R⁴ is independently Cl, F or Br;
each R⁸ is independently halogen, methyl, halomethyl or methoxy; and
each R⁹ is independently halogen, methyl, CF₃ or methoxy.

4. A compound of claim 3 wherein
Q¹ is a phenyl ring substituted at the 2-, 4- and 6-positions with substituents independently selected from R⁴; or a phenyl ring substituted at the 2- and 4-positions with substituents independently selected from R⁴; or a phenyl ring substituted at the 2- and 6-positions with substituents independently selected from R⁴;
R³ is C₁-C₆ alkyl, C₁-C₆ haloalkyl, C₂-C₆ alkenyl, C₂-C₆ alkoxyalkyl or —(CH₂)ₙW; or C₃-C₆ cycloalkyl, C₃-C₆ cycloalkenyl or C₄-C₇ cycloalkylalkyl, each optionally substituted with up to 1 substituent selected from R⁸; and
W is a 5- to 6-membered saturated or partially unsaturated heterocyclic ring containing ring members selected from carbon atoms and 1 to 2 heteroatoms independently selected from up to 2 O, up to 2 S and up to 2 N atoms, the ring optionally substituted with up to 2 substituents independently selected from R⁹ on carbon atom ring members and R¹⁰ on nitrogen atom ring members.

5. A compound of claim 1 wherein
Q¹ is a phenyl ring substituted at the 2-, 4- and 6-positions with substituents independently selected from R⁴; or a phenyl ring substituted at the 2- and 4-positions with substituents independently selected from R⁴; or a phenyl ring substituted at the 2- and 6-positions with substituents independently selected from R⁴;
X is O, NH or CHOH;
R¹ is H;
R^{1a} is H;
R² is Br, Cl, I or C₁-C₂ alkyl;
R³ is C₁-C₆ alkyl, C₁-C₆ haloalkyl, C₂-C₆ alkenyl; or C₃-C₆ cycloalkyl, C₃-C₆ cycloalkenyl or C₄-C₇ cycloalkylalkyl, each optionally substituted with up to 1 substituent selected from R⁸;
each R⁴ is independently cyano, halogen, methyl, methoxy or C₂-C₄ alkynyloxy; and
each R⁸ is halogen, methyl, halomethyl or methoxy.

6. A compound of claim 5 wherein
X is NH or CHOH;
R² is methyl;
R³ is C₁-C₆ alkyl, C₁-C₆ haloalkyl, C₂-C₆ alkenyl, C₃-C₆ cycloalkyl, C₃-C₆ cycloalkenyl or C₄-C₇ cycloalkylalkyl; and
each R⁴ is independently Cl, F or Br.

7. The compound of claim 1 which is selected from:
α-(2-chloro-4-fluorophenyl)-4-cyclohexyl-1,3-dimethyl-1H-pyrazole-5-methanol;
4-cyclohexyl-α-(2,4-difluorophenyl)-1,3-dimethyl-1H-pyrazole-5-methanol;
N-(4-chloro-2,6-difluorophenyl)-1,3-dimethyl-4-(1-methyl-1-propen-1-yl)-1H-pyrazol-5-amine;
N-(4-chloro-2,6-difluorophenyl)-4-cyclohexyl-1,3-dimethyl-1H-pyrazol-5-amine; and
N-(4-chloro-2,6-difluorophenyl)-4-cyclopenyl-1,3-dimethyl-1H-pyrazol-5-amine.

8. A fungicidal composition comprising (a) a compound of claim 1; and (b) at least one other fungicide.

9. A fungicidal composition comprising (a) a compound of claim 1; and (b) at least one additional component selected from the group consisting of surfactants, solid diluents and liquid diluents.

10. A method for controlling plant diseases caused by fungal plant pathogens comprising applying to the plant or portion thereof, or to the plant seed, a fungicidally effective amount of a compound of claim 1.
